SEQUENCE LISTING (6 sequences)

SEQ ID NO: 1 - DNA, 20 bp, Artificial Sequence
  GRN single guide
  source (1)..(20): sequence is synthesized
gtgcccaaggaccgcggagt

SEQ ID NO: 2 - DNA, 20 bp, Artificial Sequence
  no-known target scramble control
  source (1)..(20): sequence is synthesized
ggactaagcgcaagcaccta

SEQ ID NO: 3 - DNA, 21 bp, Artificial Sequence
  GRN_F primer
  source (1)..(21): sequence is synthesized
tccagagtaagtgcctctcca

SEQ ID NO: 4 - DNA, 21 bp, Artificial Sequence
  GRN_R primer
  source (1)..(21): sequence is synthesized
tcacctccatgtcacatttca

SEQ ID NO: 5 - DNA, 19 bp, Artificial Sequence
  GAPDH_F primer
  source (1)..(19): sequence is synthesized
atggggaaggtgaaggtcg

SEQ ID NO: 6 - DNA, 22 bp, Artificial Sequence
  GAPDH_R primer
  source (1)..(22): sequence is synthesized
ggggtcattgatggcaacaata

(19) United States
(12) Patent Application Publication
(10) Pub. No.: US 2024/0150710 A1
(43) Pub. Date: May 9, 2024

(54) ASSEMBLED THREE-DIMENSIONAL CULTURES OF HUMAN NEURONS AND GLIA AND THEIR USE

(71) Applicant: The Regents of the University of California, Oakland, CA (US)

(72) Inventors: Erik M. Ullian, San Francisco, CA (US); Mark Koontz, San Francisco, CA (US); Robert Krencik, San Francisco, CA (US)

(21) Appl. No.: 18/278,719

(22) PCT Filed: Feb. 25, 2022
(86) PCT No.: PCT/US2022/017942

§ 371 Date: Aug. 24, 2023

Related U.S. Application Data
(60) Provisional application No. 63/215,179, filed on Jun. 25, 2021.
(60) Provisional application No. 63/154,305, filed on Feb. 26, 2021.

(51) Int. Cl. C12N 5/079; C12N 5/0793; C12N 9/22; G01N 33/50

(52) U.S. Cl. CPC C12N 5/0622; C12N 5/0619; C12N 9/22; G01N 33/5058; C12N 2310/20; C12N 2501/11; C12N 2501/115; C12N 2501/13; C12N 2501/40; C12N 2503/04; C12N 2506/08; C12N 2513/00

(57) ABSTRACT

Compositions and methods are provided for generation of assembled three-dimensional organoids with defined numbers and ratios of mature neurons and mature glia. Organoids can be assembled from mature neurons and mature glia derived from induced pluripotent stem cells having at least one genetic mutation associated with a neurological disorder, a neurodevelopmental disorder, or a neurodegenerative disease. Such organoids can be used in disease modeling and drug screening. In particular, assembled three-dimensional organoids are provided that model granulin (GRN) loss of function in neurons and astrocytes, which display many of the pathological features of neuronal ceroid lipofusis and frontotemporal dementia.

### CROSS-REFERENCE TO RELATED APPLICATION

This application claims benefit under 35 U.S.C. § 119(e) of provisional application 63/154,305, filed Feb. 26, 2021 and provisional application 63/215,179, filed Jun. 25, 2021, which applications are hereby incorporated by reference in their entireties.

### STATEMENT REGARDING FEDERALLY SPONSORED RESEARCH OR DEVELOPMENT

This invention was made with government support under grant no. R03 AG063157 awarded by The National Institutes of Health. The government has certain rights in the invention.

### BACKGROUND OF THE INVENTION

Models of human neurodegenerative disease that recapitulate pathological features seen in patients have been a major impediment to progress in the field (Dawson et al., Nat Neurosci 21, 1370-1379, (2018); Deep-brain stimulation of the subthalamic nucleus or the pars interna of the globus pallidus in Parkinson's disease. N Engl J Med 345, 956-963, (2001)). While mouse models have provided invaluable insight into disease mechanisms for multiple neurodegenerative diseases, certain diseases such as frontotemporal lobular dementia (FTLD) have been challenging to model with characteristic pathological features (Kao et al., Nat Rev Neurosci 18, 325-333, (2017)). Recent advances in stem cell approaches have yielded models of Alzheimer's disease, showing remarkable pathological features, including Abeta accumulation and Tau pathology (Choi et al., Nature 515, 274-278, (2014); Gonzalez et al., Mol Psychiatry 23, 2363-2374, (2018); Kim et al., Nat Protoc 10, 985-1006, (2015); Kwak et al., Nat Commun 11, 1377, (2020); Lee et al., PLoS One 11, e0163072, (2016); Yan et al., Tissue Eng Part A 24, 1125-1137, (2018); Penney et al., Mol Psychiatry 25, 148-167, (2020); Hernandez-Sapiens et al., Front Cell Neurosci 14, 151, (2020); Slanzi et al., A., Front Cell Dev Biol 8, 328, (2020); Choi et al., Mol Neurodegener 11, 75, (2016); Wu et al., Open Biol 9, 180177, (2019); Mofazzal Jahromi et al., Mol Neurobiol 56, 8489-8512, (2019); Bordoni et al., Int J Mol Sci 19, (2018); Korhonen et al., Neurochem Int 120, 191-199, (2018); Centeno et al., Mol Neurodegener 13, 27, (2018); Poon et al., N Biotechnol 39, 190-198, (2017); Lee et al., J Biomed Sci 24, 59, (2017)). Similar advances have been made in other neurodegenerative disorders such as amyotrophic lateral sclerosis (ALS) and Parkinson's disease (Son et al., Neuropathol Appl Neurobiol 43, 584-603, (2017); Kim et al., Stem Cell Reports 12, 518-531, (2019); Miguel et al., Stem Cell Res 40, 101541, (2019); Bolognin et al., Adv Sci (Weinh) 6, 1800927, (2019)). To date, however, there have not been human models of FTLD that recapitulate key pathological features with loss of function mutations (Kao et al., supra; Arai et al., Biochem Biophys Res Commun 351, 602-611, (2006); Neumann et al., Science 314, 130-133, (2006); Vatsavayai et al., Acta Neuropathol 137, 1-26, (2019)). Thus, there remains a need for better models of neurodegenerative diseases.

### SUMMARY OF THE INVENTION

Compositions and methods are provided for generation of assembled three-dimensional organoids with defined numbers and ratios of mature neurons and glia. Organoids can be assembled from mature neurons and glia derived from induced pluripotent stem cells having at least one genetic mutation associated with a neurological disorder, a neurodevelopmental disorder, or a neurodegenerative disease. Such organoids can be used in disease modeling and drug screening. In particular, assembled three-dimensional organoids are provided that model granulin (GRN) loss of function in neurons and astrocytes, which display many of the pathological features of neuronal ceroid lipofusis and frontotemporal dementia.

In one aspect, a method of producing an assembled three-dimensional organoid comprising mature neurons and mature astrocytes is provided, the method comprising: a) isolating mature induced pluripotent stem cell (IPSC)-derived neurons from a first cell population and isolating mature IPSC-derived glia from a second cell population; b) combining a selected number of the mature IPSC-derived neurons and the mature IPSC-derived glia to produce a mixed culture having the mature IPSC-derived neurons and the mature IPSC-derived glia at a selected ratio; c) aggregating the mature IPSC-derived neurons and the mature IPSC-derived glia; and d) culturing the aggregated IPSC-derived neurons and IPSC-derived glia, wherein the culturing results in generation of the assembled three-dimensional organoid.

In certain embodiments, the mature IPSC-derived neurons are interneurons, motor neurons, sensory neurons, afferent neurons, efferent neurons. inhibitory neurons, or excitatory neurons, or any combination thereof.

In certain embodiments, the mature IPSC-derived neurons are glutamatergic neurons, cholinergic neurons, GABAergic neurons, dopaminergic neurons, serotonergic neurons, or histaminergic neurons, or any combination thereof.

In certain embodiments, the mature IPSC-derived neurons are produced by a method comprising: a) pre-differentiating IPSCs in pre-differentiation media comprising master neuronal transcriptional regulator neurogenin-2 (NGN2) and a rho-associated protein kinase (ROCK) inhibitor (e.g., Y-27632), wherein pre-differentiated neurons are produced; and b) culturing the pre-differentiated neurons in maturation media comprising brain-derived neurotrophic factor (BDNF) and neurotrophin-3 (NT3), wherein mature IPSC-derived neurons are produced.

In certain embodiments, the mature IPSC-derived glia are astrocytes, oligodendrocytes, ependymal cells, NG2 glia, or microglia, or any combination thereof.

In certain embodiments, the mature IPSC-derived astrocytes are produced by a method comprising: a) differentiating IPSCs into neuroepithelial cells in neural media comprising a ROCK inhibitor, wherein the neuroepithelial cells aggregate into embryo bodies; b) differentiating neuroepithelial cells in astrocyte condition media comprising epidermal growth factor (EGF) and basic fibroblast growth factor (FGFβ), wherein astrospheres comprising astrocyte progenitor cells are produced; and c) maturing astrocyte progenitor cells by culturing astrospheres in the astrocyte condition media for at least 9 months, wherein mature IPSC-derived astrocytes are produced.

In certain embodiments, the mature IPSC-derived neurons and/or the mature IPSC-derived glia comprise at least one genetic mutation associated with a neurological disorder, a neurodevelopmental disorder, or a neurodegenerative disease. In some embodiments, at least one genetic mutation is a GRN mutation associated with frontotemporal dementia or lipofusis, such as a genetic mutation that results in knockdown or knockout of a GRN gene.

In certain embodiments, the method further comprises using a CRISPR system to make genetic changes to a gene of interest in the mature IPSC-derived neurons or the mature IPSC-derived glia, or the IPSCs or progenitor cells from which they are derived. In some embodiments, the CRISPR system is used to knockdown or knockout a GRN gene in the mature IPSC-derived neurons or the mature IPSC-derived glia. In some embodiments, the glia comprise mature IPSC-derived astrocytes with knockdown or knockout of a GRN gene. In some embodiments, the CRISPR system comprises a GRN guide RNA (gRNA) comprising the sequence of SEQ ID NO:1, or a gRNA having up to three nucleotide changes in the nucleotide sequence of SEQ ID NO:1, wherein the gRNA is capable of hybridizing to a target GRN gene sequence.

In certain embodiments, the method further comprises: a) collecting somatic cells from a patient having at least one genetic mutation associated with a neurological disorder, a neurodevelopmental disorder, or a neurodegenerative disease; b) generating induced pluripotent stem cells (IPSCs) from the somatic cells; and c) differentiating the IPSCs to produce the first cell population comprising the mature IPSC-derived neurons or the second cell population comprising the mature IPSC-derived glia, or both the first cell population comprising the mature IPSC-derived neurons and the second cell population comprising the mature IPSC-derived glia.

Various somatic cells may be used to generate the IPSCs, including, without limitation, fibroblasts, keratinocytes, epithelial cells, endothelial progenitor cells, peripheral blood mononuclear cells, leukocytes, hematopoietic stem cells, mesenchymal stem cells, bone marrow cells, hepatocytes, and the like.

In certain embodiments, the selected ratio of the mature IPSC-derived neurons to the mature IPSC-derived glia is a 2:1, 1:1, 1:2, 1:3, or 1:4 ratio.

In certain embodiments, the culturing is performed in a non-adherent container.

In certain embodiments, the ratio of the mature IPSC-derived neurons and the mature IPSC-derived glia is selected to mimic the ratio of neurons and glia found in a brain region of interest.

In certain embodiments, the numbers of the mature IPSC-derived neurons and the mature IPSC-derived glia in the assembled three-dimensional organoid are selected to mimic numbers of neurons and glia found in a brain region of interest.

In certain embodiments, the mature IPSC-derived neurons and the mature IPSC-derived glia comprise types of neurons and glia found in the same brain region of interest.

In certain embodiments, the brain region of interest is in the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, or motor cortex region of the brain.

In certain embodiments, the assembled three-dimensional organoid comprises at least two types of mature IPSC-derived neurons.

In certain embodiments, the assembled three-dimensional organoid comprises at least two types of mature IPSC-derived glia.

In another aspect, an assembled three-dimensional organoid produced by a method described herein is provided.

In another aspect, a method of screening a candidate agent to determine its effects on neurons and glia is provided, the method comprising: contacting an assembled three-dimensional organoid, produced as described herein, with the candidate agent, and determining the effects of the agent on morphologic, genetic, or functional parameters.

In certain embodiments, the mature IPSC-derived neurons or the mature IPSC-derived glia in the three-dimensional organoid comprise at least one genetic mutation associated with a neurological disorder, a neurodevelopmental disorder, or a neurodegenerative disease. In some embodiments, at least one genetic mutation is a GRN mutation associated with frontotemporal dementia or lipofusis, such as a genetic mutation that results in knockdown or knockout of a GRN gene.

In certain embodiments, the mature IPSC-derived neurons are interneurons, motor neurons, sensory neurons, afferent neurons, efferent neurons. inhibitory neurons, or excitatory neurons, or a combination thereof.

In certain embodiments, the mature IPSC-derived neurons are glutamatergic neurons, cholinergic neurons, GABAergic neurons, dopaminergic neurons, serotonergic neurons, or histaminergic neurons, or a combination thereof.

In certain embodiments, the mature IPSC-derived glia are astrocytes, oligodendrocytes, ependymal cells, NG2 glia, or microglia, or a combination thereof.

In certain embodiments, determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements (e.g., patch clamping), migration assays, axonal growth and pathfinding assays, or phagocytosis assays.

In certain embodiments, the method further comprises using optogenetics to excite or inhibit one or more selected neurons of interest using light.

In another aspect, a method of producing an assembled three-dimensional organoid disease model of Parkinson's disease is provided, the method comprising: a) isolating mature induced pluripotent stem cell (IPSC)-derived dopaminergic neurons from a first cell population, isolating mature IPSC-derived astrocytes from a second cell population, and isolating mature IPSC-derived microglia from a third cell population wherein the mature IPSC-derived dopaminergic neurons, the mature IPSC-derived astrocytes, or the mature IPSC-derived microglia, or a combination thereof, comprise one or more genetic mutations associated with Parkinson's disease; b) combining a selected number of the mature IPSC-derived dopaminergic neurons, the mature IPSC-derived astrocytes, and the mature IPSC-derived microglia to produce a mixed culture having the mature IPSC-derived dopaminergic neurons, the mature IPSC-derived astrocytes, and the mature IPSC-derived microglia at a selected ratio; c) aggregating the mature IPSC-derived dopaminergic neurons, the mature IPSC-derived astrocytes, and the mature IPSC-derived microglia; and d) culturing the aggregated mature IPSC-derived dopaminergic neurons, the mature IPSC-derived astrocytes, and the mature IPSC-derived microglia, wherein the culturing results in generation of the assembled three-dimensional organoid disease model of Parkinson's disease.

In certain embodiments, the mature IPSC-derived astrocytes have ventral midbrain astrocyte characteristics.

In certain embodiments, the mature IPSC-derived dopaminergic neurons or the IPSC-derived microglia, or both the mature IPSC-derived dopaminergic neurons and the IPSC-derived microglia have midbrain characteristics.

In certain embodiments, one or more genetic mutations associated with Parkinson's disease comprise one or more mutations in one or more genes selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C. In some embodiments, the one or more genetic mutations associated with Parkinson's disease comprise an SNCA A53T mutation, an ATP13A2 c1306 mutation, or a SYNJ1 R219Q mutation.

In certain embodiments, a CRISPR system is used to introduce one or more genetic mutations associated with Parkinson's disease into the genome of the IPSC-derived dopaminergic neurons, the mature IPSC-derived astrocytes, or the mature IPSC-derived microglia, or the IPSCs or progenitor cells from which they are derived. For example, a CRISPR system can be used to knockdown or knockout a gene selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C in the IPSC-derived dopaminergic neurons, the mature IPSC-derived astrocytes, or the mature IPSC-derived microglia.

In some embodiments, the CRISPR system comprises a guide RNA (gRNA) capable of hybridizing to a target site in a SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, or VPS13C gene sequence.

In certain embodiments, mature IPSC-derived dopaminergic neurons, the mature IPSC-derived astrocytes, or the mature IPSC-derived microglia, or a combination thereof, are generated from IPSCs comprising the one or more genetic mutations associated with Parkinson's disease.

In certain embodiments, the method further comprises: a) collecting somatic cells from a patient having one or more genetic mutations associated with Parkinson's disease; b) generating IPSCs from the somatic cells; and c) differentiating the IPSCs to produce the first cell population comprising the mature IPSC-derived dopaminergic neurons, the second cell population comprising the mature IPSC-derived astrocytes, or the third cell population comprising the mature IPSC-derived microglia, or a combination thereof.

In certain embodiments, the mature IPSC-derived dopaminergic neurons, the mature IPSC-derived astrocytes, and the mature IPSC-derived microglia are generated from IPSCs derived from cells from the same source.

In certain embodiments, the selected ratio of the mature IPSC-derived dopaminergic neurons to the mature IPSC-derived astrocytes is a 2:1, 1:1, 1:2, 1:3, or 1:4 ratio.

In certain embodiments, culturing is performed in a non-adherent container.

In certain embodiments, aggregating comprises centrifuging the mixed culture.

In certain embodiments, the ratio of the mature IPSC-derived dopaminergic neurons, the mature IPSC-derived astrocytes, and the mature IPSC-derived microglia is selected to mimic the ratio of dopaminergic neurons, astrocytes, and microglia found in a midbrain region of interest.

In certain embodiments, the numbers of the mature IPSC-derived dopaminergic neurons, the mature IPSC-derived astrocytes, and the mature IPSC-derived microglia in the assembled three-dimensional organoid are selected to mimic numbers of dopaminergic neurons, astrocytes, and microglia found in a midbrain region of interest.

In certain embodiments, the midbrain region of interest comprises a substantia nigra region.

In another aspect, an assembled three-dimensional organoid disease model of Parkinson's disease produced by a method described herein is provided.

In another aspect, a method of screening a candidate agent for treatment of Parkinson's disease is provided, the method comprising: contacting the assembled three-dimensional organoid disease model of Parkinson's disease described herein with the candidate agent, and determining the effects of the agent on morphologic, genetic, or functional parameters.

In certain embodiments, determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

In certain embodiments, the method further comprises using optogenetics to excite or inhibit one or more selected dopaminergic neurons of interest using light.

In certain embodiments, the method further comprises measuring levels of dopamine or alpha-synuclein in the assembled three-dimensional organoid in presence and absence of the candidate agent.

In certain embodiments, the candidate agent is an antiglutamatergic agent, a monoamine oxidase inhibitor, a promitochondrial agent, a calcium channel blocker, or a growth factor.

In another aspect, a method of producing an assembled three-dimensional organoid disease model of Alzheimer's disease is provided, the method comprising: a) isolating mature induced pluripotent stem cell (IPSC)-derived neurons from a first cell population and isolating mature IPSC-derived glia from a second cell population, wherein the mature IPSC-derived neurons or the mature IPSC-derived glia, or the combination thereof comprise one or more genetic mutations associated with Alzheimer's disease; b) combining a selected number of the mature IPSC-derived neurons and the mature IPSC-derived glia to produce a mixed culture having the mature IPSC-derived neurons and the mature IPSC-derived glia at a selected ratio; c) aggregating the mature IPSC-derived neurons and the mature IPSC-derived glia; and d) culturing the aggregated mature IPSC-derived neurons and the mature IPSC-derived glia, wherein the culturing results in generation of the assembled three-dimensional organoid disease model of Alzheimer's disease.

In certain embodiments, the mature IPSC-derived neurons comprise cholinergic neurons.

In certain embodiments, the mature IPSC-derived glia comprise astrocytes, microglia, NG2 glia, or oligodendrocytes, or any combination thereof.

In certain embodiments, the mature IPSC-derived neurons and the mature IPSC-derived glia have hippocampus, entorhinal cortex, cerebral cortex, neocortex, amygdala, or temporal lobe characteristics.

In certain embodiments, the one or more genetic mutations associated with Alzheimer's disease comprise one or more mutations in one or more genes selected from APP, PSEN1, PSEN2, ABCA7, SORL, APOE, and TREM2. In some embodiments, the one or more genetic mutations associated with Alzheimer's disease comprise frameshift or missense mutations in APP, PSEN1, PSEN2, ABCA7, SORL, APOE, or TREM2.

In certain embodiments, a CRISPR system is used to introduce one or more genetic mutations associated with Alzheimer's disease into the genome of the IPSC-derived neurons or the mature IPSC-derived glia, or the IPSCs or progenitor cells from which they are derived. In some embodiments, the CRISPR system is used to knockdown or knockout a gene selected from APP, PSEN1, PSEN2, ABCA7, SORL, APOE, or TREM2 in the IPSC-derived neurons or the mature IPSC-derived glia, or the combination thereof. In some embodiments, the CRISPR system comprises a guide RNA (gRNA) capable of hybridizing to a target site in an APP, PSEN1, PSEN2, ABCA7, SORL, APOE, or TREM2 gene sequence. In some embodiments, the CRISPR system is used to introduce a missense or frameshift mutation in APP, PSEN1, or PSEN1.

In certain embodiments, the mature IPSC-derived neurons or the mature IPSC-derived glia are generated from IPSCs comprising the one or more genetic mutations associated with Alzheimer's disease.

In certain embodiments, the method further comprises: a) collecting somatic cells from a patient having one or more genetic mutations associated with Alzheimer's disease; b) generating IPSCs from the somatic cells; and c) differentiating the IPSCs to produce the first cell population comprising the mature IPSC-derived neurons and the second cell population comprising the mature IPSC-derived glia.

In certain embodiments, the patient has one or more mutations in one or more genes selected from APP, PSEN1, PSEN2, ABCA7, SORL, APOE, and TREM2. In some embodiments, the patient has an APOE E4 allele. In some embodiments, the patient has a missense or frameshift mutation in APP, PSEN1, or PSEN1.

In certain embodiments, the mature IPSC-derived neurons and the mature IPSC-derived glia are generated from IPSCs derived from cells from the same source.

In certain embodiments, the selected ratio of the mature IPSC-derived neurons to the mature IPSC-derived glia is a 2:1, 1:1, 1:2, 1:3, or 1:4 ratio.

In certain embodiments, culturing is performed in a non-adherent container.

In certain embodiments, aggregating comprising centrifuging the mixed culture.

In certain embodiments, the ratio of the mature IPSC-derived neurons and the mature IPSC-derived glia is selected to mimic the ratio of mature neurons and glia found in a brain region of interest.

In certain embodiments, the numbers of the mature IPSC-derived neurons and the mature IPSC-derived glia in the assembled three-dimensional organoid are selected to mimic numbers of neurons and glia found in a brain region of interest. In some embodiments, the brain region of interest comprises a hippocampus, entorhinal cortex, cerebral cortex, neocortex, amygdala, or temporal lobe region.

In another aspect, an assembled three-dimensional organoid disease model of Alzheimer's disease produced by a method described herein is provided.

In another aspect, a method of screening a candidate agent for treatment of Alzheimer's disease is provided, the method comprising: contacting the assembled three-dimensional organoid disease model of Alzheimer's disease described herein with the candidate agent, and determining the effects of the agent on morphologic, genetic, or functional parameters.

In certain embodiments, determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

In certain embodiments, the method further comprises using optogenetics to excite or inhibit one or more selected neurons of interest using light.

In certain embodiments, method further comprises measuring levels of amyloid-beta, tau, hyperphosphorylated tau, presenilins, or acetylcholine in the assembled three-dimensional organoid in presence and absence of the candidate agent.

In certain embodiments, method further comprises measuring neurofibrillary tangles inside cell bodies of the mature IPSC-derived neurons of the assembled three-dimensional organoid.

In certain embodiments, the method further comprises measuring amyloid plaques in the assembled three-dimensional organoid.

In certain embodiments, the candidate agent is a acetylcholinesterase inhibitor or an N-methyl-D-aspartate (NMDA) receptor antagonist.

### BRIEF DESCRIPTION OF THE DRAWINGS

FIGS. 1A-1E. Confirmation of GRN KO iNeurons and iAstrocytes. FIG. 1A. Schematic GRN −/− iPSCs generation. FIG. 1B. Examples of immunostains of 2 weeks old GRN +/+ iPSC induced neurons, DAPI (Blue), PGRN (Grey), β-TubulinIII (Green), scale bar=10 μm. FIG. 1C. Examples of immunostains of 2 weeks old GRN −/− iPSC induced neurons, DAPI (Blue), PGRN (Grey), β-TubulinIII (Green), scale bar=10 μm. FIG. 1D. Examples of immunostains of 10 months old), GRN +/+ iPSC induced astrocytes, DAPI (Blue), PGRN (Grey), GFAP (Green), scale bar=10 μm. FIG. 1E. Examples of immunostains of 10 months old GRN −/− iPSC induced astrocytes, DAPI (Blue), PGRN (Grey), GFAP (Green), scale bar=10 μm.

FIGS. 2A-2D. TDP43 and Ataxin2 in GRN +/+ and GRN −/− iNeurons. FIG. 2A. Examples of immunostains of 2 weeks old GRN +/+ iPSC induced neurons, DAPI (Blue), TDP43 (Grey), β-TubulinIII (Magenta), scale bar=10 μm. FIG. 2B. TDP43 intranuclear/extranuclear fluorescent comparison between GRN +/+ and GRN −/− iPSC induced neurons. (Mean±SEM, n=9-11, 3 independent experiments, t-test). FIG. 2C. Examples of immunostains of 2 weeks old GRN +/+ iPSC induced neurons, DAPI (Blue), Ataxin2 (Grey), β-TubulinIII (Magenta), scale bar=10 μm. FIG. 2D. Ataxin2 corrected total cell fluorescence (CTCF) comparison between GRN +/+ and GRN −/− iPSC induced neurons. (Mean±SEM, n=9-11, of 3 independent experiments, t-test).

FIGS. 3A-3D. Characterization of GRN +/+ and GRN −/− iAstrocytes. FIG. 3A. Examples of bright field images of 10 months old GRN +/+ and GRN −/− iPSC induced astrocytes, scale bar=100 μm. FIG. 3B. Examples of immunostains of 10 months old GRN +/+ and GRN −/− iPSC induced astrocytes, DAPI (Blue), TDP43 (Green), Ataxin2 (Red), GFAP (Magenta), scale bar=20 μm. FIG. 3C. Schematics drawing of mechanism of phagocytic activity assay. Zymosan conjugated with Phrodo Zymosan becomes fluorescent only after phagocytosed by astrocytes. Representative histogram of Phorodo Zymosan fluorescence. FIG. 3D. Quantification of Median Fluorescent Intensity (MFI) for Phrodo Zymosan 6 individual experiments between GRN +/+ and GRN −/− astrocytes. (Mean±SEM, 1-2 million cells for each experiment, 7 repeats with paired t-test).

FIGS. 4A-4G. Generation and characterization of 4 weeks and 2 weeks 3D co-culture between GRN +/+ and GRN −/− iNeurons and iAstrocytes. FIG. 4A. Schematics of 3D iNeuron and iAstrocyte co-culture set-up and how cryostat section was generated. FIG. 4B. Examples of immunostaining of cryostat section from GRN +/+ and GRN −/− iN and iA co-culture in 3D for 30 days. DAPI (Blue), TDP43 (Green), Ataxin2 (Red). Arrow indicates examples of TDP43 partially left nuclei; Asterisk indicates examples of TDP43 completely depleted from nuclei; arrowhead indicates examples of TDP43 left nuclei and form extra nuclear aggregation. FIG. 4C. TDP43 inside/outside DAPI signal volume occupancy between GRN +/+ and GRN −/− iN and iA co-culture in 3D for 30 days (Mean±SEM, n=8-11 assembloids, 3 independent experiments, t-test). FIG. 4D. Ataxin2 particle numbers per nuclei comparison between GRN +/+ and GRN −/− iN and iA co-culture in 3D for 30 days. (Mean±SEM, n=8-11 assembloids, 3 independent experiments). FIG. 4E. Examples of immunostaining of cryostat section from GRN +/+ and GRN −/− iN and iA co-culture in 3D for 15 days. DAPI (Blue), TDP43 (Green), Ataxin2 (Red). Arrow indicates examples of TDP43 partially left nuclei; Line indicates examples of TDP43 completely depleted from nuclei; arrowhead indicates examples of TDP43 left nuclei and form extra nuclear aggregation. FIG. 4F. TDP43 inside/outside DAPI signal volume occupancy between GRN +/+ and GRN −/− iN and iA co-culture in 3D for 15 days (Three colors represent three individual experiments). (Mean±SEM, n=8-11 assembloids, 3 independent experiments). FIG. 4G. Ataxin2 particle numbers per nuclei comparison between GRN +/+ and GRN −/− iN and iA co-culture in 3D for 15 days (Three colors represent three individual experiments). (Mean±SEM, n=8-11 assembloids, 3 independent experiments, t-test).

FIGS. 5A-5E. Generation and characterization of GRN knock down iNeurons and characterization of 4 weeks 3D co-culture between scramble iNeurons with GRN +/+ iAstrocytes and GRN knock down iNeurons and GRN −/− iAstrocytes. FIG. 5A. Examples of immunostains of 2 weeks old Scramble single guide iPSC induced neurons, DAPI (Blue), PGRN (Grey), β-TubulinIII (Magenta), scale bar=10 μm. FIG. 5B. Examples of immunostains of 2 weeks old GRN Knock Down guide iPSC induced neurons, DAPI (Blue), PGRN (Grey), β-TubulinIII (Magenta), scale bar=10 μm. FIG. 5C. Examples of immunostaining of cryostat section from Scramble iN with WT iA and GRN Knock Down iN and GRN −/− iA co-culture in 3D for 30 days. DAPI (Blue), TDP43 (Green), Ataxin2 (Grey). Arrow indicates examples of Ataxin2 signals. FIG. 5D. TDP43 inside/outside DAPI signal volume occupancy between Scramble iN with WT iA and GRN Knock Down iN and GRN −/− iA co-culture in 3D for 30 days. (Mean±SEM, n=8-11 assembloids, 3 independent experiments). FIG. 5E. Ataxin2 signal comparison between Scramble iN with WT iA and GRN Knock Down iN and GRN −/− iA co-culture in 3D for 30 days. (Mean±SEM, n=8-11 assembloids, 3 independent experiments, t-test).

FIGS. 6A-6H. iNeurons and iAstrocytes of GRN +/+ and GRN −/− in 2D co-culture show no difference in TDP43 localization. FIG. 6A. Immunostains of 3 weeks old 2D co-culture of GRN +/+ iN and GRN +/+ iA, DAPI (Blue), TDP43 (Green), S100b (Red), β-TubulinIII (Magenta), scale bar=20 μm. FIG. 6B. Quantification for TDP43 nuclear/cytoplasmic ratio comparison between GRN +/+ and GRN −/− Neurons from 2D co-culture. (Mean±SEM, n=5-8 individual cell, t-test). FIG. 6C. Quantification for TDP43 nuclear/cytoplasmic ratio comparison between GRN +/+ and GRN −/− iAstrocytes from 2D co-culture. (Mean±SEM, n=5-8 individual cell, t-test). FIG. 6D. Immunostains of GRN +/+ iA within 3 weeks old 2D co-culture of GRN −+/−+ iN. FIG. 6E. Examples of immunostains of GRN +/+ iN within 3 weeks old 2D co-culture of GRN +/+ iN and GRN +/+ iA, DAPI (Blue), TDP43 (Green), β-TubulinIII (Magenta), scale bar=10 μm. FIG. 6F. Examples of immunostains of GRN −+/−+ iA, DAPI (Blue), TDP43 (Green), S100b (Red), scale bar=10 μm. FIG. 6G. Examples of immunostains of GRN −/− iN within 3 weeks old 2D co-culture of GRN −/− iN and GRN −/− iA, DAPI (Blue), TDP43 (Green), β-TubulinIII (Magenta), scale bar=10 μm. Examples FIG. 6H. Examples of immunostains of GRN −/− iA within 3 weeks old 2D co-culture of GRN −/− iN and GRN −/− iA, DAPI (Blue), TDP43 (Green), S100b (Red), scale bar=10 μm.

FIGS. 7A-7C. Neurite lysosomes increase in GRN −/− iNeurons. FIG. 7A. Examples of immunostains of 2 weeks old GRN +/+ iPSC iN (induced neurons), DAPI (Blue), LAMP1 (Green), PGRN (Red), β-TubulinIII (Magenta), scale bar=10 μm. FIG. 7B. Quantification of Neurite LAMP1 signals between GRN +/+ and GRN −/− iNeurons. (Mean±SEM, n=8-11 cells, 3 independent experiments, t-test). FIG. 7C. Examples of immunostains of 2 weeks old GRN −/− iPSC iN (induced neurons), DAPI (Blue), LAMP1 (Green), PGRN (Red), β-TubulinIII (Magenta), scale bar=10 μm. Arrows indicate LAMP1 signals at Neurite region.

FIGS. 8A-8D. Ataxin2 shows no difference in 2D coculture between GRN +/+ iNeurons, iAstrocytes and GRN −/− iNeurons, iAstrocytes. FIG. 8A. Examples of immunostains of GRN +/+ iN within 3 weeks old 2D co-culture of GRN +/+ iN and GRN +/+ iA, DAPI (Blue), TDP43 (Green), Ataxin2 (Red), β-TubulinIII (Magenta), scale bar=10 μm. Examples of immunostains of GRN −/− iN within 3 weeks old 2D co-culture of GRN −/− iN and GRN −/− iA, DAPI (Blue), TDP43 (Green), Ataxin2 (Red), scale bar=10 μm. FIG. 8B. Quantification of Ataxin2 corrected total cell fluorescence between GRN +/+ iN and GRN −/− iN, n>5, t-test FIG. 8C. Examples of immunostains of GRN +/+ within 3 weeks old 2D co-culture of GRN +/+ iN and GRN +/+ iA, DAPI (Blue), TDP43 (Green), Ataxin2 (Red), β-TubulinIII (Magenta), scale bar=10 μm. Examples of immunostains of GRN −/− iA within 3 weeks old 2D co-culture of GRN −/− iN and GRN −/− iA, DAPI (Blue), TDP43 (Green), Ataxin2 (Red), scale bar=10 μm. FIG. 8D Quantification of Ataxin2 corrected total cell fluorescence between GRN +/+ iA and GRN −/− iA, n=4, t-test

FIGS. 9A-9D. Western Blotting of GRN +/+, Scramble, GRN −/− and GRN KD iPSC and quantitative analysis of GRN mRNA between Scramble and GRN KD. FIG. 9A. Western Blots of iPSCs, 3 independent experiments. FIG. 9B. RT PCR GRN quantification of 3 independent experiments of comparing scramble iNeuron and GRN Knock Down iNeurons. FIG. 9C. Examples of PGRN immunostaining of PGRN of scramble iNeurons. FIG. 9D. Examples of PGRN immunostaining of PGRN of GRN Knock Down iNeurons. Arrow indicates examples of residual PGRN within GRN Knock Down iNeurons population. (4 in around 120 iNeurons).

FIGS. 10A-10C. Neurite lysosomes increase in GRN Knock Down iNeurons compared with Scramble iNeurons. FIG. 10A. Examples of immunostains of 2 weeks old Scramble iN (induced neurons), DAPI (Blue), LAMP1 (Green), PGRN (Red), β-TubulinIII (Magenta), scale bar=10 μm. FIG. 10B. Examples of immunostains of 2 weeks old GRN Knock Down iN (induced neurons), DAPI (Blue), LAMP1 (Green), PGRN (Red), R-TubulinIII (Magenta), scale bar=10 μm. Arrows indicate LAMP1 signals at neurite region. FIG. 10C. Quantification of neurite LAMP1 signals between GRN +/+ and GRN −/− iNeurons. (Mean±SEM, n=8-11 cells, 3 independent experiments, t-test).

FIGS. 11A-11B. Gating strategies for GRN +/+ and GRN −/− iAstrocyte Zymosan Phorodo phagocytosis analysis FIG. 11A. Gating for individual live GRN +/+ iAstrocytes phagocytosed Zymosan, FIG. 11B. Gating for individual live GRN −/− iAstrocytes phagocytosed Zymosan.

FIGS. 12A-12E. Incorporation of microglia into 3D mAssembloid. FIG. 12A. Example of iPSC-derived microglia in 2D conventional culture. FIG. 12B. Incorporation of iPSC-derived microglia into mAssembloid in imaged with conventional phase light microscopy. Microglia appear as dark spots in mAssembloid (arrows). FIG. 12C. Immunostaining of mAssembloid for neuronal Tau (red) and microglial IBA1 (green) 24 hours after addition of microglia. Note numerous microglia in mAssembloid extending processes. A microglia cell can be seen migrating into the mAssembloid (arrow). FIG. 12D. Example of 30 micron section of mAssembloid 30 days after microglia incorporation. Note numerous cell bodies and processes seen in section. FIG. 12E. Computer reconstruction of a microglial cell in a 30 micron section. Note numerous processes seen in section and resting-like morphology of microglial cell indicating non-activated state.

FIG. 13. Graph showing increased Cryptic STM2 in GRN KO mAssembloids at 4 weeks in vitro. Graph is quantitation of cryptic STM2 signal relative to isogenic WT mAssembloids. Each dot represents a separate experimental run of 20 pooled mAssembloids for RNA extraction and qPCR. The cryptic form of STM2 is absent from WT mAssembloids but is significantly (Student T-test) upregulated in isogenic GRN KO mAssembloids. Note, cryptic STM2 is a recently identified biomarker of human neurodegeneration involving loss of nuclear TDP-43 and is absent from mouse models of neurodegeneration.

FIGS. 14A-14B. Assembloids showed increased phospho-TDP-43 in a GRN FTD disease model. FIG. 14A. Quantification of phospho-TDP-43/TDP-43 ratio in 4 separate runs of assembloids from WT or isogenic GRN KO assembloids of neurons+astrocytes. FIG. 14B. Immunostaining of TDP-43, phospho-TDP-43 and DAPI in WT or isogenic GRN KO assembloids showing increased phospho-TDP-43 in GRN KO assembloids. Increased phospho-TDP-43 is a hallmark of human neurodegenerative disease but is not found in animal models and has not been reported in other stem cell models.

FIGS. 15A-15B. Assembloids showed high reproducibility among separate experiments. FIG. 15A. The coefficient of variation of gene expression in separate culture runs of GRN WT and KO assembloids showed highly correlated gene expression among separate experiments indicative of high reproducibility. FIG. 15B. Heat map of gene expression showed similar direction of gene expression changes among three separate runs of isogenic WT and GRN KO assembloids again indicating high reproducibility using the assembloid method.

FIG. 16. Patient-derived assembloids containing FTD/ALS mutation bearing neurons showed cryptic STM2 expression indicating TDP-43 pathology. Results for control WT astrocyte and neuron assembloids, WT astrocyte and patient TBK1 mutation bearing neurons, GRN KO astrocytes and ALS patient neurons, and the standard GRN KO astrocyte and GRN KO neuron assembloids are shown. All combinations of disease assembloids showed cryptic STM2 in assembloids.

### DETAILED DESCRIPTION OF THE INVENTION

Compositions and methods are provided for generation of assembled three-dimensional organoids with defined numbers and ratios of mature neurons and glia. Organoids can be assembled from mature neurons and glia derived from induced pluripotent stem cells having at least one genetic mutation associated with a neurological disorder, a neurodevelopmental disorder, or a neurodegenerative disease. Methods of using such organoids in disease modeling and drug screening are also described. In particular, assembled three-dimensional organoids are provided that model GRN loss of function in neurons and astrocytes, which display many of the pathological features of neuronal ceroid lipofusis and frontotemporal dementia.

Before the present compositions and methods are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the induced pluripotent stem cell” includes reference to one or more induced pluripotent stem cells and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, or hepatocytes.

As used herein, “reprogramming factors” refers to one or more, i.e., a cocktail, of biologically active factors that act on a cell to alter transcription, thereby reprogramming a cell to multipotency or to pluripotency. Reprogramming factors may be provided individually or as a single composition, that is, as a premixed composition, of reprogramming factors to the cells, e.g., somatic cells from an individual with a family history or genetic make-up of interest, such as a patient who has a neurological disorder or a neurodegenerative disease. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

The somatic cells may include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, or hepatocytes, etc., which are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Differentiation of IPSCs into neurons (IPSC-derived neurons) and glia (IPSC-derived glia) may be promoted by using lineage-determining transcription factors and various growth factors and other differentiation agents. For example, the master neuronal transcriptional regulator neurogenin-2 (NGN2) in combination with brain-derived neurotrophic factor (BDNF) and neurotrophin-3 (NT3) promotes differentiation of iPSCs into glutamatergic cortical neurons. Expression of the transcription factors Islet-1 (ISL1) and LIM Homeobox 3 (LHX3) along with NGN2 promotes differentiation of IPSCs into motor neurons. Expression of the transcription factors achaete-scute family bHLH transcription factor 1 (ASCL1) and distal-less homeobox 2 (DLX2) induces the generation of GABAergic neurons. Growth factors such as epidermal growth factor (EGF) and basic fibroblast growth factor (FGFb) can be used to promote differentiation of IPSCs into astrocytes. Macrophage colony stimulating factor 1 (MCSF), interleukin-34 (IL-34), transforming growth factor beta 1 (TGFβ-1), CD200 and C-X3-C motif chemokine ligand 1 (CX3CL1) can be used to promote differentiation of IPSCs into microglia. Ventral midbrain astrocytes can be generated with Chir99021 and purmorphamine. Dopaminergic neurons can be generated using the proneural factor Ascl1 in combination with mesencephalic factors Lmx1a and Nurr1. Co-delivery of additional midbrain transcription factors En1, FoxA2, and Pitx3 produces dopaminergic neurons having midbrain characteristics. See, e.g., Examples and differentiation protocols described by Reyes et al. (2008) J Neurosci. 28(48):12622-12631, Zhang et al. (2013) Neuron 78(5):785-98, Hester et al. (2011) Mol. Ther. 19:1905-1912, Fernandopulle et al. (2018) Curr. Protoc. Cell Biol. 79(1):e51, Krencik et al. (2011) Nat. Protoc. 6:1710-1717, Yang et al. (2017) Nat. Methods 14:621-628, Muffat et al. (2016) Nat. Med. 22:1358-1367, Pandya et al. (2017) Nat. Neurosci. 20:753-759, Douvaras et al. (2017) Stem Cell Reports 8:1516-1524, Abud et al. (2017) Neuron 94, 278-293 e279, Abud et al. (2017) Neuron 94(2):278-293.e9, Krencik et al. (2011) Nat. Protoc. 6(11):1710-1717, Ng et al. (2021) Stem Cell Reports 16(7):1763-1776; herein incorporated by reference in their entireties. However, any suitable method of inducing differentiation of IPSCs into neurons and glia may be used.

The IPSC-derived neurons and glia are harvested at an appropriate stage of development, which may be determined based on the expression of markers and phenotypic characteristics of the desired mature differentiated cell type. Cultures may be empirically tested by staining for the presence of the markers of interest, by morphological determination, etc. for example, astrocytes can be identified by markers specific for cells of the astrocyte lineage, including, without limitation, GFAP, ALDH1L1, AQP4, and EAAT1-2. Neurons can be identified by markers, including without limitation, enolase 2/NSE, NeuN, MAP2, beta-Ill tubulin, neurofilament light, neurofilament medium, neurofilament heavy, and GAP-43. The mature IPSC-derived neurons and glia may be purified prior to assembly into organoids by positive selection for one or more markers expressed on mature neurons or glia, respectively. The cells are optionally enriched before or after the positive selection step by drug selection, panning, density gradient centrifugation, etc. In addition, a negative selection can be performed, where the selection is based on expression of one or more of the markers found on the somatic cells they are derived from (e.g., PBMCs, fibroblasts, epithelial cells, endothelial progenitor cells, leukocytes, hematopoietic stem cells, mesenchymal stem cells, bone marrow cells, hepatocytes), or neural progenitor cells, glial progenitor cells, neuroepithelial cells, and the like. Selection may utilize panning methods, magnetic particle selection, particle sorter selection, and the like.

The somatic cells, the IPSCs derived therefrom, or the mature IPSC-derived neurons or glia may be genetically modified for a variety of purposes, e.g., to introduce a genetic mutation associated with a neurological disorder, a neurodevelopmental disorder, or a neurodegenerative disease, inhibit expression of a gene, provide marker genes, etc. Vectors may be introduced that express an exogenous gene, reprogramming factors, CRISPR systems, antisense nucleic acids, or ribozymes. Various techniques known in the art may be used to introduce nucleic acids into the target cells, e.g., electroporation, calcium precipitated DNA, fusion, transfection, lipofection, infection and the like. The particular manner in which the DNA is introduced is not critical to the practice of the invention.

By “container” is meant a glass, plastic, or metal vessel that can provide an aseptic environment for culturing cells.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Substantially purified” generally refers to isolation of a substance (compound, protein, nucleic acid, nanoparticles) such that the substance comprises the majority percent of the sample in which it resides. Typically in a sample, a substantially purified component comprises 50%, preferably 80%-85%, more preferably 90-95% of the sample. Techniques for purifying substances of interest are well-known in the art and include, for example, ion-exchange chromatography, affinity chromatography and sedimentation according to density.

The terms “subject”, “individual” or “patient” are used interchangeably herein and refer to a vertebrate, preferably a mammal. By “vertebrate” is meant any member of the subphylum Chordata, including, without limitation, humans and other primates, including nonhuman primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

A “CRISPR system” refers collectively to transcripts and other elements involved in the expression of or directing the activity of CRISPR-associated (“Cas”) genes. In some embodiments, one or more elements of a CRISPR system is derived from a type I, type II, or type Ill CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks).

A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

By “selectively binds” with reference to a guide RNA is meant that the guide RNA binds preferentially to a target sequence of interest or binds with greater affinity to the target sequence than to other genomic sequences. For example, a gRNA will bind to a substantially complementary sequence and not to unrelated sequences. A gRNA that “selectively binds” to a particular allele, such as a particular mutant allele (e.g., allele comprising a substitution, insertion, or deletion), denotes a gRNA that binds preferentially to the particular target allele, but to a lesser extent to a wild-type allele or other sequences. A gRNA that selectively binds to a particular target DNA sequence will selectively direct binding of Cas9 to a substantially complementary sequence at the target site and not to unrelated sequences.

The term “donor polynucleotide” refers to a polynucleotide that provides a sequence of an intended edit to be integrated into the genome at a target locus by homology directed repair (HDR).

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele).

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5 genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. The nucleotide sequence comprising the intended edit is integrated into the genomic DNA by HDR or recombineering at the genomic target locus recognized (i.e., sufficiently complementary for hybridization) by the 5′ and 3′ homology arms.

“Administering” a nucleic acid, such as an inhibitory or regulatory nucleic acid (e.g., microRNA, siRNA, piRNA, snRNA, antisense nucleic acid, or lncRNA), or a CRISPR system (expressing, e.g., a donor polynucleotide, guide RNA, Cas protein (e.g., Cas9, Cas12a, Cas12d, Cas13, or dCas9)) to a cell comprises transducing, transfecting, electroporating, translocating, fusing, phagocytosing, shooting or ballistic methods, etc., i.e., any means by which a nucleic acid can be transported across a cell membrane.

### Assembling Organoids Containing Mature Neurons and Glia

Methods are provided for generation of assembled three-dimensional organoids with defined numbers and ratios of mature neurons and mature glia. Organoids are assembled from separate populations of mature neurons and mature glia derived from IPSCs. Assembling organoids from mature cells has the advantage that it provides precise control over the types of cells and the maturity of the cells in the organoid. Neurons and astrocytes typically take significant amounts of time to differentiate from IPSCs. Astrocytes, in particular, take long periods of time, as much as 9 months, to mature in culture. As a result, other coculture systems typically contain a poorly defined population of cells with a mixture of progenitor and mature neuronal subtypes. The organoids, disclosed herein, because they are assembled from mature neurons and glia, better mimic the natural nervous system environment and can be used to provide improved models of the nervous system under normal and disease conditions.

The mature IPSC-derived neurons and the mature IPSC-derived glia can be generated by reprogramming somatic cells into pluripotent stem cells followed by redifferentiation into neurons and glia, respectively. Somatic cells can be induced into forming pluripotent stem cells, for example, by treating them with reprograming factors such as Yamanaka factors, including but not limited to, OCT3, OCT4, SOX2, KLF4, c-MYC, NANOG, and LIN28 (see, e.g., Takahashi et al. (2007) Cell. 131(5):861-872; herein incorporated by reference in its entirety). The types of somatic cells that may be converted into IPSCs include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes. Somatic cells are contacted with reprogramming factors in a combination and quantity sufficient to reprogram the cells to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Methods for “introducing a cell reprogramming factor into somatic cells are not limited in particular, and known procedures can be selected and used as appropriate. For example, when a cell reprogramming factor as described above is introduced into somatic cells of the above-mentioned type in the form of proteins, such methods include ones using protein introducing reagents, fusion proteins with protein transfer domains (PTDs), electroporation, and microinjection. When a cell reprogramming factor as described above is introduced into somatic cells of the above-mentioned type in the form of nucleic acids encoding the cell reprogramming factor, a nucleic acid(s), such as cDNA(s), encoding the cell reprogramming factor can be inserted in an appropriate expression vector comprising a promoter that functions in somatic cells, which then can be introduced into somatic cells by procedures such as infection, lipofection, liposomes, electroporation, calcium phosphate coprecipitation, DEAE-dextran, microinjection, and electroporation. Examples of an “expression vector” include viral vectors, such as lentiviruses, retroviruses, adenoviruses, adeno-associated viruses, and herpes viruses; and expression plasmids for animal cells. For example, retroviral or Sendai virus (SeV) vectors are commonly used to introduce a nucleic acid(s) encoding a cell reprogramming factor as described above into somatic cells.

In some embodiments the IPSCs are derived from somatic cells obtained from neurologically normal individuals. In other embodiments the IPSCs are derived from somatic cells obtained from an individual comprising at least one allele encoding a mutation associated with a neurological disorder, neurodevelopmental disorder, or neurodegenerative disease associated with neuron/glia dysfunction, including, without limitation, neuronal ceroid lipofusis, frontotemporal dementia, Rett syndrome, fragile X mental retardation, Alexander's disease, amyotrophic lateral sclerosis (ALS), Niemann-Pick type C disease, neurofibromatosis type-1, Noonan syndrome, Leopard syndrome, cardiofaciocutaneous (CFC) syndrome, Costello syndrome, schizophrenia, autism spectrum disorders, epilepsy, multiple sclerosis, leukodystrophies, anti-AQP4+ neuromyelitis optica, Rasmussen's encephalitis, Alzheimer's disease, Parkinson's disease, Huntington's disease, Charcot-Marie-Tooth disease, Myasthenia gravis, and chronic pain.

A sample comprising somatic cells is obtained from the subject. The somatic cells may include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes, and other cell types capable of generating patient-derived IPSCs that can be differentiated into mature neurons or glia, The biological sample comprising somatic cells is typically whole blood, buffy coat, peripheral blood mononucleated cells (PBMCS), skin, fat, or a biopsy, but can be any sample from bodily fluids, tissue or cells that contain suitable somatic cells. A biological sample can be obtained from a subject by conventional techniques. For example, blood can be obtained by venipuncture, and solid tissue samples can be obtained by surgical techniques according to methods well known in the art.

Differentiation of IPSCs into neurons (IPSC-derived neurons) and glia (IPSC-derived glia) may be promoted by using lineage-determining transcription factors and various growth factors and other differentiation agents. For example, the master neuronal transcriptional regulator neurogenin-2 (NGN2) in combination with brain-derived neurotrophic factor (BDNF) and neurotrophin-3 (NT3) promotes differentiation of iPSCs into glutamatergic cortical neurons. Expression of the transcription factors Islet-1 (ISL1) and LIM Homeobox 3 (LHX3) along with NGN2 promotes differentiation of IPSCs into motor neurons. Expression of the transcription factors achaete-scute family bHLH transcription factor 1 (ASCL1) and distal-less homeobox 2 (DLX2) induces the generation of GABAergic neurons. Growth factors such as epidermal growth factor (EGF) and basic fibroblast growth factor (FGFb) can be used to promote differentiation of IPSCs into astrocytes. Macrophage colony stimulating factor 1 (MCSF), interleukin-34 (IL-34), transforming growth factor beta 1 (TGFβ-1), CD200 and C-X3-C motif chemokine ligand 1 (CX3CL1) can be used to promote differentiation of IPSCs into microglia. Ventral midbrain astrocytes can be generated with Chir99021 and purmorphamine. Dopaminergic neurons can be generated using the proneural factor Ascl1 in combination with mesencephalic factors Lmx1a and Nurr1. Co-delivery of additional midbrain transcription factors En1, FoxA2, and Pitx3 produces dopaminergic neurons having midbrain characteristics. See, e.g., the Examples and differentiation protocols described by Reyes et al. (2008) J Neurosci. 28(48):12622-12631, Zhang et al. (2013) Neuron 78(5):785-98, Hester et al. (2011) Mol. Ther. 19:1905-1912, Fernandopulle et al. (2018) Curr. Protoc. Cell Biol. 79(1):e51, Krencik et al. (2011) Nat. Protoc. 6:1710-1717, Yang et al. (2017) Nat. Methods 14:621-628, Muffat et al. (2016) Nat. Med. 22:1358-1367, Pandya et al. (2017) Nat. Neurosci. 20:753-759, Douvaras et al. (2017) Stem Cell Reports 8:1516-1524, Abud et al. (2017) Neuron 94, 278-293 e279, Abud et al. (2017) Neuron 94(2):278-293.e9, Krencik et al. (2011) Nat. Protoc. 6(11):1710-1717, Ng et al. (2021) Stem Cell Reports 16(7):1763-1776; herein incorporated by reference in their entireties. However, any suitable method of inducing differentiation of IPSCs into neurons and glia may be used.

For example, cortical neurons can be produced from a human IPSC cell line with inducible expression of neurogenin 2 (NGN2), which promotes differentiation into cortical neurons after induction in a neural supportive medium (see, e.g., Example 1). NGN2 can be induced, for example, by addition of doxycycline to cultures. Pre-differentiated neurons, formed in the presence of NGN2, are further matured by adding brain-derived neurotrophic factor (BDNF) and neurotrophin-3 (NT3) to the culture.

Astrocytes can be produced from human IPSCs by a multi-step process (see, e.g., Example 1). First, the IPSCs are differentiated into neuroepithelial cells in a neural supportive medium comprising a ROCK inhibitor, wherein the neuroepithelial cells aggregate into embryo bodies. The neuroepithelial cells are further differentiated in an astrocyte condition medium comprising epidermal growth factor (EGF) and basic fibroblast growth factor (FGFβ), wherein astrospheres comprising astrocyte progenitor cells are produced. To mature the astrocyte progenitor cells, the astrospheres are cultured in the astrocyte condition media for at least 9 months, wherein mature IPSC-derived astrocytes are produced. Astrocytes can be generated that have characteristics of specific central nervous system regions, including the midbrain by generating neural rosettes using a combination of Chir99021 and purmorphamine, which are then differentiated to astrocytes (see, e.g., Example 3).

Microglia can be generated using a two-step protocol that starts with differentiation of IPSCs to hematopoietic progenitor cells that can be further differentiated into microglia-like cells, by growing in a defined serum-free media supplemented with the three cytokines: M-CSF1, TGFβ and IL-34 (see, e.g., Example 3).

The IPSC-derived neurons and glia are harvested at an appropriate stage of development, which may be determined based on the expression of markers and phenotypic characteristics of the desired mature differentiated cell type. Cultures may be empirically tested by staining for the presence of the markers of interest, by morphological determination, etc. for example, astrocytes can be identified by markers specific for cells of the astrocyte lineage, including, without limitation, GFAP, ALDH1L1, AQP4, and EAAT1-2. Neurons can be identified by markers, including without limitation, enolase 2/NSE, NeuN, MAP2, beta-Ill tubulin, neurofilament light, neurofilament medium, neurofilament heavy, and GAP-43. The mature IPSC-derived neurons and glia may be purified prior to assembly into organoids by positive selection for one or more markers expressed on mature neurons or glia, respectively. The cells are optionally enriched before or after the positive selection step by drug selection, panning, density gradient centrifugation, etc. In addition, a negative selection can be performed, where the selection is based on expression of one or more of the markers found on the somatic cells they are derived from (e.g., PBMCs, fibroblasts, epithelial cells, endothelial progenitor cells, leukocytes, hematopoietic stem cells, mesenchymal stem cells, bone marrow cells, hepatocytes), or neural progenitor cells, glial progenitor cells, neuroepithelial cells, and the like. Selection may utilize panning methods, magnetic particle selection, particle sorter selection, and the like.

To assemble the organoid, pre-matured neurons and glia from separate cultures are combined to form a mixed culture with defined numbers and ratios of cells. Growth factor treatment of cells is preferably stopped before coculturing to allow better control over cell numbers. Media are seeded with the pre-matured neurons and glia initially in the presence of a ROCK inhibitor (Y-27632) followed by coculturing the cells in the absence of the ROCK inhibitor. The mixed culture can be centrifuged to aggregate the mature IPSC-derived neurons and the mature IPSC-derived glia followed by culturing in a neural supportive medium to generate the assembled three-dimensional organoid (see, e.g., Example 1).

In certain embodiments, the cell-types, numbers, and ratios of the neurons and glia, which are combined to produce the organoid, are chosen to mimic the cell-types, numbers, and ratios of neurons and glia found in a brain region of interest. In certain embodiments, one or more brain regions of interest are in the cerebrum, cerebellum, or brainstem regions of the brain. Brain regions of interest may include, without limitation, the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, and motor cortex regions of the brain.

In certain embodiments, the three-dimensional organoid is assembled from multiple types of neurons and/or glia. For example, the organoid may be assembled from one or more types of excitatory neurons and/or inhibitory neurons and glia found in a brain region of interest. Glia may include, without limitation, astrocytes, oligodendrocytes, ependymal cells, microglia, and NG2 glia, In some embodiments, multiple types of glia are assembled with the neurons, e.g., neurons with astrocytes and microglia, neurons with astrocytes and oligodendrocytes, neurons with astrocytes, microglia, and oligodendrocytes, or neurons with astrocytes, microglia, oligodendrocytes, and NG2 glia. In some embodiments, the ratio of the mature IPSC-derived neurons to the mature IPSC-derived glia in the mixed culture is a 2:1, 1:1, 1:2, 1:3, or 1:4 ratio.

### Genome Modification to Introduce Disease-Relevant Genetic Changes

Disease-relevant mutations can be introduced into the genome of the mature IPSC-derived neurons or the mature IPSC-derived glia, or the IPSCs or progenitor cells from which they are derived using any method known in the art to produce an assembled three-dimensional organoid disease model. In some embodiments, a CRISPR/Cas system is used to make genetic changes to a gene of interest in the mature IPSC-derived neurons or the mature IPSC-derived glia, or the IPSCs or progenitor cells from which they are derived, for example, to introduce a mutation associated with a neurological disorder, neurodevelopmental disorder, or neurodegenerative disease associated with neuron/glia dysfunction to produce an organoid useful for disease modeling and drug screening. For example, a CRISPR/Cas system can be used to delete, inactivate, or mutate a gene, or eliminate or reduce gene expression or protein activity. Genome modification can be performed, for example, using homology directed repair (HDR) with a donor polynucleotide comprising a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit is integrated into the genomic DNA by HDR at the genomic target locus recognized (i.e., sufficiently complementary for hybridization) by the 5′ and 3′ homology arms.

In certain embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In a preferred embodiment, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g., 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another, e.g., one may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g., one may be 40% shorter or more, 50% shorter or more, sometimes 60% shorter or more, 70% shorter or more, 80% shorter or more, 90% shorter or more, or 95% shorter or more than the other homology arm.

The donor polynucleotide is used in combination with an RNA-guided nuclease, which is targeted to a particular genomic sequence (i.e., genomic target sequence to be modified) by a guide RNA (gRNA). A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to a target sequence in a gene of interest. In some embodiments, the gRNA is designed with a sequence complementary to a specific mutation to target the nuclease-gRNA complex to the site of a mutation in a cell. The mutation may comprise an insertion, a deletion, or a substitution. For example, the mutation may include a single nucleotide variation, gene fusion, translocation, inversion, duplication, frameshift, missense, nonsense, or other mutation. The targeted minor allele may be a common genetic variant or a rare genetic variant. In certain embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to delete or deactivate the gene in a neuron or glia.

In certain embodiments, the RNA-guided nuclease used for genome modification is a CRISPR system Cas nuclease. Any RNA-guided Cas nuclease capable of catalyzing site-directed cleavage of DNA to allow integration of donor polynucleotides by the HDR mechanism can be used in genome editing, including CRISPR system type I, type II, or type Ill Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, Cas12a (Cpf1), Cas12b (C2c1), Cas12c (C2c3), Cas12d (CasY), Cas12e (CasX), CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisl (NC_018721); Streptococcus thermophilus (YP_820832), Streptococcus mutans (WP_061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacteriol. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a 5′ protospacer-adjacent motif (PAM) in the DNA at or near the gRNA-binding site.

The genomic target site will typically comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, also known as Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

Cas12b (C2c1) is another class II CRISPR/Cas system RNA-guided nuclease that may be used. C2c1, similarly to Cas9, depends on both a crRNA and tracrRNA for guidance to target sites. For a description of Cas12b, see, e.g., Shmakov et al. (2015) Mol Cell. 60(3):385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided FokI nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

An RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector). In some embodiments, the RNA-guided nuclease and the gRNA are both provided by vectors. Both can be expressed by a single vector or separately on different vectors. The vector(s) encoding the RNA-guided nuclease an gRNA may be included in a CRISPR expression system to target a gene of interest in neurons or glia.

Codon usage may be optimized to improve production of an RNA-guided nuclease in a particular cell or organism. For example, a nucleic acid encoding an RNA-guided nuclease or reverse transcriptase can be modified to substitute codons having a higher frequency of usage in a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells (e.g., neurons or glia), the protein can be transiently, conditionally, or constitutively expressed in the cell.

In another embodiment, CRISPR interference (CRISPRi) is used to repress gene expression. CRISPRi is performed with a complex of a catalytically inactive Cas9 (dCas9) with a guide RNA that targets the gene of interest. An engineered nuclease-deactivated Cas9 (dCas9) is used to allow sequence-specific targeting without cleavage. Nuclease-deactivated forms of Cas9 may be engineered by mutating catalytic residues at the active site of Cas9 to destroy nuclease activity. Any such nuclease deficient Cas9 protein from any species may be used as long as the engineered dCas9 retains gRNA-mediated sequence-specific targeting. In particular, the nuclease activity of Cas9 from Streptococcus pyogenes can be deactivated by introducing two mutations (D10A and H841A) in the RuvC1 and HNH nuclease domains. Other engineered dCas9 proteins may be produced by similarly mutating the corresponding residues in other bacterial Cas9 isoforms. For a description of engineered nuclease-deactivated forms of Cas9, see, e.g., Qi et al. (2013) Cell 152:1173-1183, Dominguez et al. (2016) Nat. Rev. Mol. Cell. Biol. 17(1):5-15; herein incorporated by reference in their entireties.

The dCas9 protein can be designed to target a gene of interest by altering its guide RNA sequence. A target-specific single guide RNA (sgRNA) comprises a nucleotide sequence that is complementary to a target site, and thereby mediates binding of the dCas9-sgRNA complex by hybridization at the target site. CRISPRi can be used to sterically repress transcription by blocking either transcriptional initiation or elongation by designing a sgRNA with a sequence complementary to a promoter or exonic sequence. The sgRNA may be complementary to the non-template strand or the template strand, but preferably is complementary to the non-template strand to more strongly repress transcription.

The target site will typically comprise a nucleotide sequence that is complementary to the sgRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide.

In certain embodiments, the sgRNA comprises 5-50 nucleotides, 10-30 nucleotides, 15-25 nucleotides, 18-22 nucleotides, 19-21 nucleotides, and any length between the stated ranges, including, for example, 10, 11, 12, 13, 14, 15, 16, 17, 18, 19, 20, 21, 22, 23, 24, 25, 26, 27, 28, 29, or 30 nucleotides.

The sgRNAs are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109.

In some embodiments, the dCas9 is fused to a transcriptional repressor domain capable of further repressing transcription of the gene of interest, e.g., by inducing heterochromatinization. For example, a Krüppel associated box (KRAB) can be fused to dCas9 to repress transcription of a target gene in human cells (see, e.g., Gilbert et al. (2013) Cell. 154 (2): 442-45, O'Geen et al. (2017) Nucleic Acids Res. 45(17):9901-9916; herein incorporated by reference).

Alternatively, dCas9 can be used to introduce epigenetic changes that reduce expression of a gene of interest by fusion of dCas9 to an epigenetic modifier such as a chromatin-modifying epigenetic enzyme. The promoter for the gene of interest can be silenced, for example, by methylation or acetylation (e.g., histone H3 lysine 9 [H3K9] methylation, histone H3 lysine 27 [H3K27]methylation, and/or DNA methylation). For example, fusion of dCas9 to a DNA methyltransferase such as DNA methyltransferase 3 alpha (DNMT3A) or a chimeric Dnmt3a/Dnmt3L methyltransferase (DNMT3A3L) allows targeted DNA methylation. Fusion of dCas9 to histone demethylase LSD1 allows targeted histone demethylation (see, e.g., Liu et al. (2016) Cell 167(1):233-247, Lo et al. (2017) F1000Res. 6. pii: F1000 Faculty Rev-747, and Stepper et al. (2017) Nucleic Acids Res. 45(4):1703-1713; herein incorporated by reference).

In yet other embodiments, an RNA-targeting CRISPR-Cas13 system is used to perform RNA interference to reduce expression of a gene of interest. Members of the Cas13 family are RNA-guided RNases containing two HEPN domains having RNase activity. In particular, Cas13a (C2c2), Cas13b (C2c6), and Cas13d can be used for RNA knockdown. Cas13 proteins can be made to target and cleave transcribed RNA using a gRNA with complementarity to the target transcript sequence. The gRNA is typically about 64 nucleotides in length with a short hairpin crRNA and a 28-30 nucleotide spacer that is complementary to the target site on the RNA transcript. Cas13 recognition and cleavage of a target transcript results in degradation of the transcript as well as nonspecific degradation of any nearby transcripts. See, e.g., Abudayyeh et al. (2017) Nature 550:280-284, Hameed et al. (2019) Microb. Pathog. 133:103551, Wang et al. (2019) Biotechnol Adv. 37(5):708-729, Aman et al. (2018) Viruses 10(12). pii: E732, and Zhang et al. (2018) Cell 175(1):212-223; herein incorporated by reference.

In certain embodiments, a CRISPR system is used to knockdown or knockout a GRN gene in the mature IPSC-derived neurons and/or the mature IPSC-derived glia (or the IPSCs or progenitor cells they are derived from) to produce an organoid that can be used as a disease model of neuronal ceroid lipofusis or frontotemporal dementia. In some embodiments, the glia comprise mature IPSC-derived astrocytes with knockdown or knockout of a GRN gene. In some embodiments, the CRISPR system comprises a GRN guide RNA (gRNA) comprising the sequence of SEQ ID NO:1, or a gRNA having up to three nucleotide changes in the nucleotide sequence of SEQ ID NO:1, wherein the gRNA is capable of hybridizing to a target GRN gene sequence.

In certain embodiments, a CRISPR system is used to introduce one or more mutations linked to Parkinson's disease into the mature IPSC-derived neurons and/or the mature IPSC-derived glia (or the IPSCs or progenitor cells they are derived from) to produce an organoid that can be used as a disease model of Parkinson's disease. Mutations in the SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C genes have been linked to development of Parkinson's disease. A 22q11 deletion is also known to be associated with Parkinson's disease. Mutations in the GBA1 gene, which encodes glucocerebrosidase, increase the risk of developing Parkinson's disease by 20-fold to 30-fold. Exemplary mutations linked to Parkinson's disease include, without limitation, mutations in LRRK2 such as G2019S, mutations in GBA1 such as E326K and T369 M, mutations in SNCA such as A53T and E46K, mutations in SYNJ1 such as R219Q, and mutations in the ATP13A2 gene such as c1306, c.1510, c.1597, c1632, and c3057 mutations. Additional representative mutations linked to Parkinson's disease are listed in the Parkinson Disease Mutation Database (molgen.vib-ua.be/PDMutDB/), Gene4PD (genemed.tech/gene4pd/home), and PDbase (bioportal.kobic.re.kr/PDbase/); all of which mutations (as entered by the date of filing of this application) are herein incorporated by reference.

In certain embodiments, a CRISPR system is used to introduce one or more mutations linked to Alzheimer's disease into the mature IPSC-derived neurons and/or the mature IPSC-derived glia (or the IPSCs or progenitor cells they are derived from) to produce an organoid that can be used as a disease model of Alzheimer's disease. Mutations in the genes encoding amyloid-beta precursor protein (APP) and presenilins, including presenilin 1 (PSEN1) and presenilin 2 (PSEN2) have been linked to Alzheimer's disease. Mutations in ABCA7 and SORL are also linked to Alzheimer's disease. Mutations in APOE (e.g., APOE E4 allele increases risk for Alzheimer's disease) and TREM2 increase the risk of developing Alzheimer's disease. Exemplary mutations linked to Alzheimer's disease include, without limitation, mutations in APP such as V717L, S198P, and A235V, mutations in PSEN1 such as W165C, T119I, and A79V, and mutations in PSEN2 such as T430M, V214L, and F369S. Missense and frameshift variants in APP, PSEN1, and PSEN1 are associated with Alzheimer's disease. Additional representative mutations linked to Alzheimer's disease are listed in the Alzheimer Disease & Frontotemporal Dementia Mutation Database (molgen.vib-ua.be/ADMutations) and Alzforum databases (alzforum.org/databases); all of which mutations (as entered by the date of filing of this application) are herein incorporated by reference.

### Screening Assays

Organoids can be subjected to a plurality of candidate agents or other therapeutic intervention. Candidate agents include, without limitation, small molecules, i.e., drugs, genetic constructs that increase or decrease expression of an RNA of interest, CRISPR systems, optogenetic perturbation, electrical changes, and the like. Methods are also provided for determining the activity of a candidate agent on a disease-relevant cell, the method comprising contacting one or more cells of the organoid comprising at least one allele encoding a mutation associated with a neurological disorder, neurodevelopmental disorder, or neurodegenerative disease associated with neuron/glia dysfunction with the candidate agent; and determining the effect of the agent on morphologic, genetic or functional parameters. In screening assays for the small molecules, the effect of contacting the organoid with the candidate agent to determine the effect on migration, synapse formation, etc. in culture is tested with one or a panel of cellular environments, where the cellular environment includes one or more of: electrical stimulation including alterations in ionicity, stimulation with a candidate agent of interest, contact with other cells including without limitation neurons and neural progenitors, contact with infectious agents, e.g. bacterial, viral, fungal, or parasitic infectious agents, and the like, and where cells may vary in genotype, in prior exposure to an environment of interest, in the dose of agent that is provided, etc. Usually at least one control is included, for example, a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional and genetic changes.

Examples of analytic methods comprise, for example, assessing the synaptic integration of migrated neurons by using array tomography to detect pre- and post-synaptic proteins. To further examine synaptic puncta ‘synaptograms’ consisting of a series of high-resolution sections through a single synapse may be obtained. Electrophysiology measurements including voltage clamp recordings of synaptic responses can be performed on slices of the organoid.

Live imaging of cells, including during cell migration, may be performed and cells modified to express a detectable marker. Calcium sensitive dyes can be used, e.g., Fura-2 calcium imaging; Fluo-4 calcium imaging, GCaMP6 calcium imaging, voltage imaging using voltage indicators such as voltage-sensitive dyes (e.g., di-4-ANEPPS, di-8-ANEPPS, and RH237) and/or genetically-encoded voltage indicators (e.g., ASAP1, Archer) can be used on the intact organoids, or on cells isolated therefrom.

Methods of analysis at the single cell level are also of interest, e.g., as described above: live imaging (including confocal or light-sheet microscopy), single cell gene expression or single cell RNA sequencing, calcium imaging, immunocytochemistry, patch-clamping, flow cytometry and the like. Various parameters can be measured to determine the effect of a drug or treatment on the organoid or cells derived therefrom.

Parameters include quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. Although most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g., polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases, the molecular entities comprising the epitope are from two or more substances and comprise a defined structure; examples include combinatorically determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. In particular, candidate drugs, select therapeutic antibodies and protein-based therapeutics with preferred biological response functions can be evaluated. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, neurotransmitters, hormones or hormone antagonists, ion channel modifiers, and neuroactive agents. Exemplary pharmaceutical agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds may include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g., ground water, sea water, mining waste, etc.; biological samples, e.g., lysates prepared from crops, tissue samples, etc.; manufacturing samples, e.g., time course during preparation of pharmaceuticals; as well as libraries of compounds prepared for analysis; and the like. Samples of interest include compounds being assessed for potential therapeutic value, i.e., drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g., under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g., morpholino oligonucleotide analogs.

Agents are screened for biological activity by adding the agent to at least one and usually a plurality of cells, in one or in a plurality of environmental conditions, e.g., following stimulation with an agonist, following electric or mechanical stimulation, etc. The change in parameter readout in response to the agent is measured, desirably normalized, and the resulting screening results may then be evaluated by comparison to reference screening results, e.g., with cells having other mutations of interest, normal astrocytes, astrocytes derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus, preferred formulations consist essentially of a biologically active compound and a physiologically acceptable carrier, e.g., water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to fluoresce, e.g., by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

Of particular interest for the disclosed neuronal screening system are parameters related to the electrical properties of the cells and therefore directly informative about neuronal function and activity. Methods to measure neuronal activity may sense the occurrence of action potentials (spikes). The characteristics of the occurrence of a single spike or multiple spikes either in timely clustered groups (bursts) or distributed over longer time (spike train) of a single neuron or a group of neurons indicate neuronal activation patterns and thus reflect functional neuronal properties, which can be described my multiple parameters. Such parameters can be used to quantify and describe changes in neuronal activity.

Neuronal activity parameters include, without limitation, total number of spikes (per recording period); mean firing rate (of spikes); inter-spike interval (distance between sequential spikes); total number of bursts (per recording period); burst frequency; number of spikes per burst; burst duration (in milliseconds); inter-burst interval (distance between sequential bursts); burst percentage (the portion of spikes occurring within a burst); total number of network bursts (spontaneous synchronized network activity); network burst frequency; number of spikes per network burst; network burst duration; inter-network-burst interval; inter-spike interval within network bursts; network burst percentage (the portion of bursts occurring within a network burst); salutatory migration, etc.

Quantitative readouts of neuronal activity parameters may include baseline measurements in the absence of agents or a pre-defined genetic control condition and test measurements in the presence of a single or multiple agents or a genetic test condition. Furthermore, quantitative readouts of neuronal activity parameters may include long-term recordings and may therefore be used as a function of time (change of parameter value). Readouts may be acquired either spontaneously or in response to or presence of stimulation or perturbation of the complete neuronal network or selected components of the network. The quantitative readouts of neuronal activity parameters may further include a single determined value, the mean or median values of parallel, subsequent or replicate measurements, the variance of the measurements, various normalizations, the cross-correlation between parallel measurements, etc. and every statistic used to a calculate a meaningful and informative factor.

Comprehensive measurements of neuronal activity using electrical or optical recordings of the parameters described herein may include spontaneous activity and activity in response to targeted electrical or optical stimulation of all neuronal cells or a subpopulation of neuronal cells within the organoid. Furthermore, spontaneous or induced neuronal activity can be measured in the self-assembled functional environment and circuitry of the neural culture or under conditions of selective perturbation or excitation of specific subpopulations of neuronal cells as discussed above.

In the provided assays, comprehensive measurements of neuronal activity can be conducted at different time points along neuronal maturation and usually include a baseline measurement directly before contacting the neural culture with the agents of interest and a subsequent measurement under agent exposure. Moreover, long-term effects of agents on neural maturation and development can be assessed by contacting the immature neural culture at an early time point with agents of interest and acquiring measurements of the same cultures after further maturation at a later time point compared to control cultures without prior agent exposure.

In some embodiments, standard recordings of neuronal activity of mature neural cultures are conducted after about 2 weeks, after about 3 weeks, after about 4 weeks, after about 6 weeks, after about 8 weeks following assembly of the organoid. Recordings of neuronal activity may encompass the measurement of additive, synergistic or opposing effects of agents that are successively applied to the cultures, therefore the duration recording periods can be adjusted according to the specific requirements of the assay. In some embodiments the measurement of neuronal activity is performed for a predetermined concentration of an agent of interest, whereas in other embodiments measurements of neuronal activity can be applied for a range of concentrations of an agent of interest.

In some embodiments, the assays described herein are used to evaluate changes in organoid function in response to optogenetic perturbation of neural activity. In certain embodiments, optogenetics is used to induce cell-specific perturbations in the organoid. For example, optogenetics can be used to excite or inhibit one or more selected neurons of interest using light. For a description of optogenetics techniques, see, e.g., Abe et al., 2012; Desai et al., 2011; Duffy et al., 2015; Gerits et al., 2012; Kahn et al., 2013; Lee et al., 2010; Liu et al., 2015; Ohayon et al., 2013; Weitz et al., 2015; Weitz and Lee, 2013; herein incorporated by reference.

In some embodiments the provided assays are used to assess maturation of the neural culture or single components including glutamatergic neurons, GABAergic interneurons, astrocytes, oligodendrocytes, microglia etc. Maturation of neuronal cells can be measured based on morphology by optically assessing parameters such as dendritic arborization, axon elongation, total area of neuronal cell bodies, number of primary processes per neuron, total length of processes per neuron, number of branching points per primary process as well as density and size of synaptic puncta stained by synaptic markers such as synapsin-1, synaptophysin, bassoon, PSD95, and Homer. Moreover, general neuronal maturation and differentiation can be assessed by measuring expression of marker proteins such as MAP2, TUJ-1, NeuN, Tau, PSA-NCAM, and SYN-1 alone or in combination using FACS analysis, immunoblotting, or fluorescence microscopy imaging, patch clamping. Maturation and differentiation of neuronal subtypes can further be tested by measuring expression of specific proteins. For excitatory neuronal cells this includes staining for e.g., VGLUT1/2, GRIA1/2/3/4, GRIN1, GRIN2A/B, GPHN etc. For inhibitory neuronal cells this includes staining for e.g., GABRA2, GABRB1, VGAT, and GAD67.

The results of an assay can be entered into a data processor to provide a dataset. Algorithms are used for the comparison and analysis of data obtained under different conditions. The effect of factors and agents is read out by determining changes in multiple parameters. The data will include the results from assay combinations with the agent(s), and may also include one or more of the control state, the simulated state, and the results from other assay combinations using other agents or performed under other conditions. For rapid and easy comparisons, the results may be presented visually in a graph, and can include numbers, graphs, color representations, etc.

The dataset is prepared from values obtained by measuring parameters in the presence and absence of different cells, e.g., genetically modified cells, cells cultured in the presence of specific factors or agents that affect neuronal function, as well as comparing the presence of the agent of interest and at least one other state, usually the control state, which may include the state without the agent or with a different agent. The parameters include functional states such as synapse formation and calcium ions in response to stimulation, whose levels vary in the presence of the factors. The results may be normalized against a standard, usually a “control value or state,” to provide a normalized data set. Values obtained from test conditions can be normalized by subtracting the unstimulated control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a dataset may comprise normalized data from one, two or multiple cell types and assay conditions.

The dataset can comprise values of the levels of sets of parameters obtained under different assay combinations. Compilations are developed that provide the values for a sufficient number of alternative assay combinations to allow comparison of values.

A database can be compiled from sets of experiments, for example, a database can contain data obtained from a panel of assay combinations, with multiple different environmental changes, where each change can be a series of related compounds, or compounds representing different classes of molecules.

Mathematical systems can be used to compare datasets, and to provide quantitative measures of similarities and differences between them. For example, the datasets can be analyzed by pattern recognition algorithms or clustering methods (e.g., hierarchical or k-means clustering, etc.) that use statistical analysis (correlation coefficients, etc.) to quantify relatedness. These methods can be modified (by weighting, employing classification strategies, etc.) to optimize the ability of a dataset to discriminate different functional effects. For example, individual parameters can be given more or less weight when analyzing the dataset, in order to enhance the discriminatory ability of the analysis. The effect of altering the weights assigned each parameter is assessed, and an iterative process is used to optimize pathway or cellular function discrimination.

The comparison of a dataset obtained from a test compound, and a reference dataset(s) is accomplished by the use of suitable deduction protocols, AI systems, statistical comparisons, etc. Preferably, the dataset is compared with a database of reference data. Similarity to reference data involving known pathway stimuli or inhibitors can provide an initial indication of the cellular pathways targeted or altered by the test stimulus or agent.

A reference database can be compiled. These databases may include reference data from panels that include known agents or combinations of agents that target specific pathways, as well as references from the analysis of cells treated under environmental conditions in which single or multiple environmental conditions or parameters are removed or specifically altered. Reference data may also be generated from panels containing cells with genetic constructs that selectively target or modulate specific cellular pathways. In this way, a database is developed that can reveal the contributions of individual pathways to a complex response.

The effectiveness of pattern search algorithms in classification can involve the optimization of the number of parameters and assay combinations. The disclosed techniques for selection of parameters provide for computational requirements resulting in physiologically relevant outputs. Moreover, these techniques for pre-filtering data sets (or potential data sets) using cell activity and disease-relevant biological information improve the likelihood that the outputs returned from database searches will be relevant to predicting agent mechanisms and in vivo agent effects.

For the development of an expert system for selection and classification of biologically active drug compounds or other interventions, the following procedures are employed. For every reference and test pattern, typically a data matrix is generated, where each point of the data matrix corresponds to a readout from a parameter, where data for each parameter may come from replicate determinations, e.g., multiple individual cells of the same type. As previously described, a data point may be quantitative, semi-quantitative, or qualitative, depending on the nature of the parameter.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e., data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like.

A knowledge database will be of sufficient complexity to permit novel test data to be effectively identified and classified. Several approaches for generating a sufficiently encompassing set of classification patterns, and sufficiently powerful mathematical/statistical methods for discriminating between them can accomplish this.

The data from cells treated with specific drugs known to interact with particular targets or pathways provide a more detailed set of classification readouts. Data generated from cells that are genetically modified using over-expression techniques and anti-sense techniques, permit testing the influence of individual genes on the phenotype.

A preferred knowledge database contains reference data for the organoids, environments and parameters. For complex environments, data reflecting small variations in the environment may also be included in the knowledge database, e.g., environments where one or more factors or cell types of interest are excluded or included or quantitatively altered in, for example, concentration or time of exposure, etc.

### Parkinson's Disease Model

In some embodiments, an assembled three-dimensional organoid disease model of Parkinson's disease is provided. The disease model of Parkinson's disease can be produced by a method comprising: a) isolating mature induced pluripotent stem cell (IPSC)-derived dopaminergic neurons from a first cell population, isolating mature IPSC-derived astrocytes from a second cell population, and isolating mature IPSC-derived microglia from a third cell population wherein the mature IPSC-derived dopaminergic neurons, the mature IPSC-derived astrocytes, or the mature IPSC-derived microglia, or a combination thereof, comprise one or more genetic mutations associated with Parkinson's disease; b) combining a selected number of the mature IPSC-derived dopaminergic neurons, the mature IPSC-derived astrocytes, and the mature IPSC-derived microglia to produce a mixed culture having the mature IPSC-derived dopaminergic neurons, the mature IPSC-derived astrocytes, and the mature IPSC-derived microglia at a selected ratio; c) aggregating the mature IPSC-derived dopaminergic neurons, the mature IPSC-derived astrocytes, and the mature IPSC-derived microglia; and d) culturing the aggregated mature IPSC-derived dopaminergic neurons, the mature IPSC-derived astrocytes, and the mature IPSC-derived microglia, wherein the culturing results in generation of the assembled three-dimensional organoid disease model of Parkinson's disease. In certain embodiments, the mature IPSC-derived astrocytes have ventral midbrain astrocyte characteristics. In certain embodiments, the mature IPSC-derived dopaminergic neurons or the IPSC-derived microglia, or both the mature IPSC-derived dopaminergic neurons and the IPSC-derived microglia have midbrain characteristics. In some embodiments, the mature IPSC-derived dopaminergic neurons, the mature IPSC-derived astrocytes, and the mature IPSC-derived microglia are generated from IPSCs derived from cells from the same source.

In certain embodiments, the selected ratio of the mature IPSC-derived dopaminergic neurons to the mature IPSC-derived astrocytes is a 2:1, 1:1, 1:2, 1:3, or 1:4 ratio. In some embodiments, the ratio of the mature IPSC-derived dopaminergic neurons, the mature IPSC-derived astrocytes, and the mature IPSC-derived microglia is selected to mimic the ratio of dopaminergic neurons, astrocytes, and microglia found in a midbrain region of interest. In certain embodiments, the numbers of the mature IPSC-derived dopaminergic neurons, the mature IPSC-derived astrocytes, and the mature IPSC-derived microglia in the assembled three-dimensional organoid are selected to mimic numbers of dopaminergic neurons, astrocytes, and microglia found in a midbrain region of interest. In certain embodiments, the midbrain region of interest comprises a substantia nigra region.

In another aspect, a method of screening a candidate agent for treatment of Parkinson's disease is provided, the method comprising: contacting the assembled three-dimensional organoid disease model of Parkinson's disease described herein with the candidate agent, and determining the effects of the agent on morphologic, genetic, or functional parameters. In certain embodiments, determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays. In certain embodiments, the method further comprises using optogenetics to excite or inhibit one or more selected dopaminergic neurons of interest using light.

In certain embodiments, the method further comprises measuring levels of dopamine or alpha-synuclein in the assembled three-dimensional organoid in presence and absence of the candidate agent.

In certain embodiments, the candidate agent is an antiglutamatergic agent, a monoamine oxidase inhibitor, a promitochondrial agent, a calcium channel blocker, or a growth factor.

### Alzheimer's Disease Model

In some embodiments, an assembled three-dimensional organoid disease model of Alzheimer's disease is provided. The disease model of Alzheimer's disease can be produced by a method comprising: a) isolating mature induced pluripotent stem cell (IPSC)-derived neurons from a first cell population and isolating mature IPSC-derived glia from a second cell population, wherein the mature IPSC-derived neurons or the mature IPSC-derived glia, or the combination thereof comprise one or more genetic mutations associated with Alzheimer's disease; b) combining a selected number of the mature IPSC-derived neurons and the mature IPSC-derived glia to produce a mixed culture having the mature IPSC-derived neurons and the mature IPSC-derived glia at a selected ratio; c) aggregating the mature IPSC-derived neurons and the mature IPSC-derived glia; and d) culturing the aggregated mature IPSC-derived neurons and the mature IPSC-derived glia, wherein the culturing results in generation of the assembled three-dimensional organoid disease model of Alzheimer's disease. In some embodiments, the mature IPSC-derived neurons comprise cholinergic neurons. In some embodiments, the mature IPSC-derived glia comprise astrocytes, microglia, NG2 glia, or oligodendrocytes, or any combination thereof. In some embodiments, the mature IPSC-derived neurons and the mature IPSC-derived glia have hippocampus, entorhinal cortex, cerebral cortex, neocortex, amygdala, or temporal lobe characteristics. In certain embodiments, the mature IPSC-derived neurons and the mature IPSC-derived glia are generated from IPSCs derived from cells from the same source.

In certain embodiments, the selected ratio of the mature IPSC-derived neurons to the mature IPSC-derived glia is a 2:1, 1:1, 1:2, 1:3, or 1:4 ratio. In certain embodiments, the ratio of the mature IPSC-derived neurons and the mature IPSC-derived glia is selected to mimic the ratio of neurons and the glia found in a brain region of interest. In certain embodiments, the numbers of the mature IPSC-derived neurons and the mature IPSC-derived glia in the assembled three-dimensional organoid are selected to mimic numbers of neurons and glia found in a brain region of interest. In some embodiments, the brain region of interest comprises a hippocampus, entorhinal cortex, cerebral cortex, neocortex, amygdala, or temporal lobe region.

In certain embodiments, the one or more genetic mutations associated with Alzheimer's disease comprise one or more mutations in one or more genes selected from APP, PSEN1, PSEN2, ABCA7, SORL, APOE, and TREM2. In some embodiments, the one or more genetic mutations associated with Alzheimer's disease comprise frameshift or missense mutations in APP, PSEN1, PSEN2, ABCA7, SORL, APOE, or TREM2. In certain embodiments, a CRISPR system is used to introduce one or more genetic mutations associated with Alzheimer's disease into the genome of the IPSC-derived neurons or the mature IPSC-derived glia, or the IPSCs or progenitor cells from which they are derived. In some embodiments, the CRISPR system is used to knockdown or knockout a gene selected from APP, PSEN1, PSEN2, ABCA7, SORL, APOE, or TREM2 in the IPSC-derived neurons or the mature IPSC-derived glia, or the combination thereof. In some embodiments, the CRISPR system comprises a guide RNA (gRNA) capable of hybridizing to a target site in an APP, PSEN1, PSEN2, ABCA7, SORL, APOE, or TREM2 gene sequence. In some embodiments, the CRISPR system is used to introduce a missense or frameshift mutation in APP, PSEN1, or PSEN1.

In certain embodiments, the method further comprises: a) collecting somatic cells from a patient having one or more genetic mutations associated with Alzheimer's disease; b) generating IPSCs from the somatic cells; and c) differentiating the IPSCs to produce the first cell population comprising the mature IPSC-derived neurons and the second cell population comprising the mature IPSC-derived glia. In some embodiments, the patient has one or more mutations in one or more genes selected from APP, PSEN1, PSEN2, ABCA7, SORL, APOE, and TREM2. In some embodiments, the patient has an APOE E4 allele. In some embodiments, the patient has a missense or frameshift mutation in APP, PSEN1, or PSEN1.

In another aspect, a method of screening a candidate agent for treatment of Alzheimer's disease is provided, the method comprising: contacting the assembled three-dimensional organoid disease model of Alzheimer's disease described herein with the candidate agent, and determining the effects of the agent on morphologic, genetic, or functional parameters. In certain embodiments, determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays. In certain embodiments, the method further comprises using optogenetics to excite or inhibit one or more selected neurons of interest using light.

In certain embodiments, method further comprises measuring levels of amyloid-beta, tau, hyperphosphorylated tau, presenilins, or acetylcholine in the assembled three-dimensional organoid in presence and absence of the candidate agent. In certain embodiments, method further comprises measuring neurofibrillary tangles inside cell bodies of the mature IPSC-derived neurons of the assembled three-dimensional organoid. In certain embodiments, the method further comprises measuring amyloid plaques in the assembled three-dimensional organoid.

In certain embodiments, the candidate agent is an acetylcholinesterase inhibitor or an N-methyl-D-aspartate (NMDA) receptor antagonist.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-88 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of producing an assembled three-dimensional organoid comprising mature neurons and mature glia, the method comprising:
a) isolating mature induced pluripotent stem cell (IPSC)-derived neurons from a first cell population and isolating mature IPSC-derived glia from a second cell population;
b) combining a selected number of the mature IPSC-derived neurons and the mature IPSC-derived glia to produce a mixed culture having the mature IPSC-derived neurons and the mature IPSC-derived glia at a selected ratio;
c) aggregating the mature IPSC-derived neurons and the mature IPSC-derived glia; and
d) culturing the aggregated IPSC-derived neurons and IPSC-derived glia, wherein the culturing results in generation of the assembled three-dimensional organoid.

2. The method of aspect 1, wherein the mature IPSC-derived neurons are interneurons, motor neurons, sensory neurons, afferent neurons, efferent neurons. inhibitory neurons, or excitatory neurons, or any combination thereof.

3. The method of aspect 1, wherein the mature IPSC-derived neurons are glutamatergic neurons, cholinergic neurons, GABAergic neurons, dopaminergic neurons, serotonergic neurons, or histaminergic neurons, or any combination thereof.

4. The method of any one of aspects 1 to 3, wherein the mature IPSC-derived neurons are produced by a method comprising:
a) pre-differentiating IPSCs in pre-differentiation media comprising master neuronal transcriptional regulator neurogenin-2 (NGN2) and a rho-associated protein kinase (ROCK) inhibitor, wherein pre-differentiated neurons are produced; and
b) culturing the pre-differentiated neurons in maturation media comprising brain-derived neurotrophic factor (BDNF) and neurotrophin-3 (NT3), wherein mature IPSC-derived neurons are produced.

5. The method of any one of aspects 1 to 4, wherein the mature IPSC-derived glia are astrocytes, oligodendrocytes, ependymal cells, microglia, NG2 glia, or any combination thereof.

6. The method of aspect 5, wherein the mature IPSC-derived astrocytes are produced by a method comprising:
a) differentiating IPSCs into neuroepithelial cells in neural media comprising a ROCK inhibitor, wherein the neuroepithelial cells aggregate into embryo bodies;
b) differentiating neuroepithelial cells in astrocyte condition media comprising epidermal growth factor (EGF) and basic fibroblast growth factor (FGFβ), wherein astrospheres comprising astrocyte progenitor cells are produced; and
c) maturing astrocyte progenitor cells by culturing astrospheres in the astrocyte condition media for at least 9 months, wherein mature IPSC-derived astrocytes are produced.

7. The method of any one of aspects 1 to 6, wherein the mature IPSC-derived neurons or the mature IPSC-derived glia or both the mature IPSC-derived neurons and the mature IPSC-derived glia comprise at least one genetic mutation associated with a neurological disorder, a neurodevelopmental disorder, or a neurodegenerative disease.

8. The method of aspect 7, wherein said at least one genetic mutation is a GRN mutation associated with frontotemporal dementia or lipofusis.

9. The method of aspect 7 or 8, where said at least one genetic mutation results in knockdown or knockout of a GRN gene.

10. The method of any one of aspects 1 to 9, further comprising using a CRISPR system to make genetic changes to a gene of interest in the mature IPSC-derived neurons or the mature IPSC-derived glia, or the IPSCs or progenitor cells from which they are derived.

11. The method of aspect 10, wherein the CRISPR system is used to knockdown or knockout a GRN gene in the mature IPSC-derived neurons or the mature IPSC-derived glia.

12. The method of aspect 11, wherein the CRISPR system comprises a GRN guide RNA (gRNA) comprising the sequence of SEQ ID NO:1, or a gRNA having up to three nucleotide changes in the nucleotide sequence of SEQ ID NO:1, wherein the gRNA is capable of hybridizing to a target GRN gene sequence.

13. The method of any one of aspects 1 to 12, wherein the mature IPSC-derived neurons or the mature IPSC-derived glia or both the mature IPSC-derived neurons and the mature IPSC-derived glia are generated from IPSCs comprising at least one genetic mutation associated with a neurological disorder, a neurodevelopmental disorder, or a neurodegenerative disease.

14. The method of any one of aspects 1 to 12, further comprising:
a) collecting somatic cells from a patient having at least one genetic mutation associated with a neurological disorder, a neurodevelopmental disorder, or a neurodegenerative disease;
b) generating IPSCs from the somatic cells; and
c) differentiating the IPSCs to produce the first cell population comprising the mature IPSC-derived neurons or the second cell population comprising the mature IPSC-derived glia, or both the first cell population comprising the mature IPSC-derived neurons and the second cell population comprising the mature IPSC-derived glia.

15. The method of any one of aspects 1 to 12, further comprising genetically modifying the mature IPSC-derived neurons or the mature IPSC-derived glia or both the mature IPSC-derived neurons and the mature IPSC-derived glia to introduce at least one genetic mutation associated with a neurological disorder, a neurodevelopmental disorder, or a neurodegenerative disease into their genome.

16. The method of any one of aspects 1 to 15, wherein the mature IPSC-derived neurons and the mature IPSC-derived glia are generated from IPSCs derived from cells from the same source.

17. The method of any one of aspects 1 to 16, wherein the selected ratio of the mature IPSC-derived neurons to the mature IPSC-derived glia is a 2:1, 1:1, 1:2, 1:3, or 1:4 ratio.

18. The method of any one of aspects 1 to 17, wherein said culturing is performed in a non-adherent container.

19. The method of any one of aspects 1 to 18, wherein said aggregating comprising centrifuging the mixed culture.

20. The method of any one of aspects 1 to 19, wherein the ratio of the mature IPSC-derived neurons and the mature IPSC-derived glia is selected to mimic the ratio of neurons and glia found in a brain region of interest.

21. The method of any one of aspects 1 to 20, wherein the numbers of the mature IPSC-derived neurons and the mature IPSC-derived glia in the assembled three-dimensional organoid are selected to mimic numbers of neurons and glia found in a brain region of interest.

22. The method of aspect 20 or 21, wherein the mature IPSC-derived neurons and the mature IPSC-derived glia comprise types of neurons and glia found in the same brain region of interest.

23. The method of any one of aspects 20 to 22, wherein the brain region of interest is in the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, or motor cortex region of the brain.

24. The method of any one of aspects 1 to 23, wherein the assembled three-dimensional organoid comprises at least two types of mature IPSC-derived neurons.

25. The method of any one of aspects 1 to 24, wherein the assembled three-dimensional organoid comprises at least two types of mature IPSC-derived glia.

26. An assembled three-dimensional organoid produced by the method of any one of aspects 1 to 25.

27. A method of screening a candidate agent to determine its effects on neurons and glia, the method comprising: contacting the assembled three-dimensional organoid of aspect 26 with the candidate agent, and determining the effects of the agent on morphologic, genetic, or functional parameters.

28. The method of aspect 27, wherein the mature IPSC-derived neurons or the mature IPSC-derived glia in the three-dimensional organoid comprise at least one genetic mutation associated with a neurological disorder, a neurodevelopmental disorder, or a neurodegenerative disease.

29. The method of aspect 28, wherein said at least one genetic mutation is a GRN mutation associated with frontotemporal dementia or lipofusis.

30. The method of aspect 28 or 29, where said at least one genetic mutation results in knockdown or knockout of a GRN gene.

31. The method of any one of aspects 27 to 30, wherein the mature IPSC-derived neurons are interneurons, motor neurons, sensory neurons, afferent neurons, efferent neurons. inhibitory neurons, or excitatory neurons, or any combination thereof.

32. The method of aspect 31, wherein the mature IPSC-derived neurons are glutamatergic neurons, cholinergic neurons, GABAergic neurons, dopaminergic neurons, serotonergic neurons, or histaminergic neurons, or any combination thereof.

33. The method of any one of aspects 27 to 32, wherein the mature IPSC-derived glia are astrocytes, oligodendrocytes, ependymal cells, NG2 glia, or microglia, or any combination thereof.

34. The method of any one of aspects 27 to 33, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

35. The method of any one of aspects 27 to 34, further comprising using optogenetics to excite or inhibit one or more selected neurons of interest using light.

36. A method of producing an assembled three-dimensional organoid disease model of Parkinson's disease, the method comprising:
a) isolating mature induced pluripotent stem cell (IPSC)-derived dopaminergic neurons from a first cell population, isolating mature IPSC-derived astrocytes from a second cell population, and isolating mature IPSC-derived microglia from a third cell population wherein the mature IPSC-derived dopaminergic neurons, the mature IPSC-derived astrocytes, or the mature IPSC-derived microglia, or a combination thereof, comprise one or more genetic mutations associated with Parkinson's disease;
b) combining a selected number of the mature IPSC-derived dopaminergic neurons, the mature IPSC-derived astrocytes, and the mature IPSC-derived microglia to produce a mixed culture having the mature IPSC-derived dopaminergic neurons, the mature IPSC-derived astrocytes, and the mature IPSC-derived microglia at a selected ratio;
c) aggregating the mature IPSC-derived dopaminergic neurons, the mature IPSC-derived astrocytes, and the mature IPSC-derived microglia; and
d) culturing the aggregated mature IPSC-derived dopaminergic neurons, the mature IPSC-derived astrocytes, and the mature IPSC-derived microglia, wherein the culturing results in generation of the assembled three-dimensional organoid disease model of Parkinson's disease.

37. The method of aspect 36, wherein the mature IPSC-derived astrocytes have ventral midbrain astrocyte characteristics.

38. The method of aspect 36 or 37, wherein the mature IPSC-derived dopaminergic neurons or the IPSC-derived microglia, or both the mature IPSC-derived dopaminergic neurons and the IPSC-derived microglia have midbrain characteristics.

39. The method of any one of aspects 36 to 38, wherein the one or more genetic mutations associated with Parkinson's disease comprise one or more mutations in one or more genes selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C.

40. The method of aspect 39, wherein the one or more genetic mutations associated with Parkinson's disease comprise an SNCA A53T mutation, an ATP13A2 c1306 mutation, or a SYNJ1 R219Q mutation.

41. The method of any one of aspects 36 to 40, wherein a CRISPR system is used to introduce one or more genetic mutations associated with Parkinson's disease into the genome of the IPSC-derived dopaminergic neurons, the mature IPSC-derived astrocytes, or the mature IPSC-derived microglia, or the IPSCs or progenitor cells from which they are derived.

42. The method of aspect 41, wherein the CRISPR system is used to knockdown or knockout a gene selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C in the IPSC-derived dopaminergic neurons, the mature IPSC-derived astrocytes, or the mature IPSC-derived microglia.

43. The method of aspect 42, wherein the CRISPR system comprises a guide RNA (gRNA) capable of hybridizing to a target site in a SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, or VPS13C gene sequence.

44. The method of any one of aspects 36 to 43, wherein the mature IPSC-derived dopaminergic neurons, the mature IPSC-derived astrocytes, or the mature IPSC-derived microglia, or a combination thereof, are generated from IPSCs comprising the one or more genetic mutations associated with Parkinson's disease.

45. The method of any one of aspects 36 to 44, further comprising:
a) collecting somatic cells from a patient having one or more genetic mutations associated with Parkinson's disease;
b) generating IPSCs from the somatic cells; and
c) differentiating the IPSCs to produce the first cell population comprising the mature IPSC-derived dopaminergic neurons, the second cell population comprising the mature IPSC-derived astrocytes, or the third cell population comprising the mature IPSC-derived microglia, or a combination thereof.

46. The method of any one of aspects 36 to 45, wherein the mature IPSC-derived dopaminergic neurons, the mature IPSC-derived astrocytes, and the mature IPSC-derived microglia are generated from IPSCs derived from cells from the same source.

47. The method of any one of aspects 36 to 46, wherein the selected ratio of the mature IPSC-derived dopaminergic neurons to the mature IPSC-derived astrocytes is a 2:1, 1:1, 1:2, 1:3, or 1:4 ratio.

48. The method of any one of aspects 36 to 47, wherein said culturing is performed in a non-adherent container.

49. The method of any one of aspects 36 to 48, wherein said aggregating comprising centrifuging the mixed culture.

50. The method of any one of aspects 36 to 49, wherein the ratio of the mature IPSC-derived dopaminergic neurons, the mature IPSC-derived astrocytes, and the mature IPSC-derived microglia is selected to mimic the ratio of dopaminergic neurons, astrocytes, and microglia found in a midbrain region of interest.

51. The method of any one of aspects 36 to 50, wherein the numbers of the mature IPSC-derived dopaminergic neurons, the mature IPSC-derived astrocytes, and the mature IPSC-derived microglia in the assembled three-dimensional organoid are selected to mimic numbers of dopaminergic neurons, astrocytes, and microglia found in a midbrain region of interest.

52. The method of aspect 50 or 51, wherein the midbrain region of interest comprises a substantia nigra region.

53. An assembled three-dimensional organoid disease model of Parkinson's disease produced by the method of any one of aspects 36 to 52.

54. A method of screening a candidate agent for treatment of Parkinson's disease, the method comprising: contacting the assembled three-dimensional organoid disease model of Parkinson's disease of aspect 53 with the candidate agent, and determining the effects of the agent on morphologic, genetic, or functional parameters.

55. The method of aspect 54, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

56. The method of aspect 54 or 55, further comprising using optogenetics to excite or inhibit one or more selected dopaminergic neurons of interest using light.

57. The method of any one of aspects 54 to 56, further comprising measuring levels of dopamine or alpha-synuclein in the assembled three-dimensional organoid in presence and absence of the candidate agent.

58. The method of any one of aspects 54 to 57, wherein the candidate agent is an antiglutamatergic agent, a monoamine oxidase inhibitor, a promitochondrial agent, a calcium channel blocker, or a growth factor.

59. A method of producing an assembled three-dimensional organoid disease model of Alzheimer's disease, the method comprising:
a) isolating mature induced pluripotent stem cell (IPSC)-derived neurons from a first cell population and isolating mature IPSC-derived glia from a second cell population, wherein the mature IPSC-derived neurons or the mature IPSC-derived glia, or the combination thereof comprise one or more genetic mutations associated with Alzheimer's disease;
b) combining a selected number of the mature IPSC-derived neurons and the mature IPSC-derived glia to produce a mixed culture having the mature IPSC-derived neurons and the mature IPSC-derived glia at a selected ratio;
c) aggregating the mature IPSC-derived neurons and the mature IPSC-derived glia; and
d) culturing the aggregated mature IPSC-derived neurons and the mature IPSC-derived glia, wherein the culturing results in generation of the assembled three-dimensional organoid disease model of Alzheimer's disease.

60. The method of aspect 59, wherein the mature IPSC-derived neurons comprise cholinergic neurons.

61. The method of aspect 59 or 60, wherein the mature IPSC-derived glia comprise astrocytes, microglia, NG2 glia, or oligodendrocytes, or any combination thereof.

62. The method of any one of aspects 59 to 61, wherein the mature IPSC-derived neurons and the mature IPSC-derived glia have hippocampus, entorhinal cortex, cerebral cortex, neocortex, amygdala, or temporal lobe characteristics.

63. The method of any one of aspects 59 to 62, wherein the one or more genetic mutations associated with Alzheimer's disease comprise one or more mutations in one or more genes selected from APP, PSEN1, PSEN2, ABCA7, SORL, APOE, and TREM2.

64. The method of aspect 63, wherein the one or more genetic mutations associated with Alzheimer's disease comprise frameshift or missense mutations in APP, PSEN1, PSEN2, ABCA7, SORL, APOE, or TREM2.

65. The method of any one of aspects 59 to 64, wherein a CRISPR system is used to introduce one or more genetic mutations associated with Alzheimer's disease into the genome of the IPSC-derived neurons or the mature IPSC-derived glia, or the IPSCs or progenitor cells from which they are derived.

66. The method of aspect 65, wherein the CRISPR system is used to knockdown or knockout a gene selected from APP, PSEN1, PSEN2, ABCA7, SORL, APOE, or TREM2 in the IPSC-derived neurons or the mature IPSC-derived glia, or the combination thereof.

67. The method of aspect 66, wherein the CRISPR system comprises a guide RNA (gRNA) capable of hybridizing to a target site in an APP, PSEN1, PSEN2, ABCA7, SORL, APOE, or TREM2 gene sequence.

68. The method of aspect 67, wherein the CRISPR system is used to introduce a missense or frameshift mutation in APP, PSEN1, or PSEN1.

69. The method of any one of aspects 59 to 68, wherein the mature IPSC-derived neurons or the mature IPSC-derived glia are generated from IPSCs comprising the one or more genetic mutations associated with Alzheimer's disease.

70. The method of any one of aspects 59 to 69, further comprising:
a) collecting somatic cells from a patient having one or more genetic mutations associated with Alzheimer's disease;
b) generating IPSCs from the somatic cells; and
c) differentiating the IPSCs to produce the first cell population comprising the mature IPSC-derived neurons and the second cell population comprising the mature IPSC-derived glia.

71. The method of aspect 70, wherein the patient has one or more mutations in one or more genes selected from APP, PSEN1, PSEN2, ABCA7, SORL, APOE, and TREM2.

72. The method of aspect 71, wherein the patient has an APOE E4 allele.

73. The method of aspect 71, wherein the patient has a missense or frameshift mutation in APP, PSEN1, or PSEN1.

74. The method of any one of aspects 59 to 73, wherein the mature IPSC-derived neurons and the mature IPSC-derived glia are generated from IPSCs derived from cells from the same source.

75. The method of any one of aspects 59 to 74, wherein the selected ratio of the mature IPSC-derived neurons to the mature IPSC-derived glia is a 2:1, 1:1, 1:2, 1:3, or 1:4 ratio.

76. The method of any one of aspects 59 to 75, wherein said culturing is performed in a non-adherent container.

77. The method of any one of aspects 59 to 76, wherein said aggregating comprising centrifuging the mixed culture.

78. The method of any one of aspects 59 to 77, wherein the ratio of the mature IPSC-derived neurons and the mature IPSC-derived glia is selected to mimic the ratio of neurons and glia found in a brain region of interest.

79. The method of any one of aspects 59 to 78, wherein the numbers of the mature IPSC-derived neurons and the mature IPSC-derived glia in the assembled three-dimensional organoid are selected to mimic numbers of neurons and glia found in a brain region of interest.

80. The method of aspect 78 or 79, wherein the brain region of interest comprises a hippocampus, entorhinal cortex, cerebral cortex, neocortex, amygdala, or temporal lobe region.

81. An assembled three-dimensional organoid disease model of Alzheimer's disease produced by the method of any one of aspects 59 to 80.

82. A method of screening a candidate agent for treatment of Alzheimer's disease, the method comprising: contacting the assembled three-dimensional organoid disease model of Alzheimer's disease of aspect 81 with the candidate agent, and determining the effects of the agent on morphologic, genetic, or functional parameters.

83. The method of aspect 82, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

84. The method of aspects 82 or 83, further comprising using optogenetics to excite or inhibit one or more selected neurons of interest using light.

85. The method of any one of aspects 82 to 84, further comprising measuring levels of amyloid-beta, tau, hyperphosphorylated tau, presenilins, or acetylcholine in the assembled three-dimensional organoid in presence and absence of the candidate agent.

86. The method of any one of aspects 82 to 85, further comprising measuring neurofibrillary tangles inside cell bodies of the mature IPSC-derived neurons of the assembled three-dimensional organoid.

87. The method of any one of aspects 82 to 86, further comprising measuring amyloid plaques in the assembled three-dimensional organoid.

88. The method of any one of aspects 82 to 87, wherein the candidate agent is a acetylcholinesterase inhibitor or an N-methyl-D-aspartate (NMDA) receptor antagonist.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Assembled 3D Cultures of Human Neurons and Astrocytes Recapitulate Key Pathological Features of Neurodegeneration

### Introduction

In this study we extend our previous work²⁸ to present a new straightforward method of co-culturing human iPSC-derived neurons and astrocytes in a highly reproducible 3D model that recapitulates key aspects of human pathology such as TDP-43 nuclear loss and extranuclear aggregation in a Granulin gene (GRN) deletion model. Autosomal dominant mutations in GRN have been linked to a familial form of frontotemporal dementia (FTD)^{3,29}). One of the key neuropathological features of FTD patients is the presence of protein aggregates in cortical neurons. The extra-nuclear aggregates are found to be enriched in TAR DNA-binding protein 43 (TDP43)^{25,26}. GRN knockout animal models have provided significant insights in characterizing the cellular roles of progranulin. Specifically, transcriptomic analysis of GRN KO mice have implicated both lysosomal pathway disfunction and excessive complement signaling as underlying mechanisms for disease³⁰. It has further been reported that GRN KO mice retinal neurons exhibit some nuclear loss of TDP43 and downregulation of GTPase Ran prior to neurodegeneration^{31, 32}. Yet, despite these advances the characteristic TDP43 pathology is largely absent from mouse cortex^{33; 34; 31}

Recent work has established the utility of iPSC models for the study of GRN loss of function (LOF)^{35, 36}. GRN LOF in human iPSC neurons show cell autonomous effects in signaling³⁷ and cellular stress³⁵. Interestingly, these studies did not find the characteristic TDP-43 pathology in human iPSC-derived neurons that is indicative of late-stage disease^{38,31}. One possible explanation for this is the fact that GRN is expressed in glia, such as microglia and astrocytes, raising the likelihood that GRN loss of function leads to pathology in a non-cell autonomous manner^{39,40,41}. Recent studies have further implicated glial cells, including astrocytes and microglia, in neurodegenerative disease progression^{42,43,44}). Indeed, several lines of evidence from GRN knockout mouse models indicate GRN deficiency induces glial complement activation and non-autonomous microglia-mediated synaptic pruning that subsequently leads to neurodegeneration³⁰. Importantly, the characteristic TDP-43 pathology of late-stage diseases is not present in these animal models despite compelling pathology. This raises the question of whether other glial cells such as astrocytes, can induce TDP-43 pathology and whether human models can show more characteristic pathology. To address these questions, we developed a novel 3D engineered approach to study human neuron-astrocyte interactions in the context of GRN LOF.

### Results

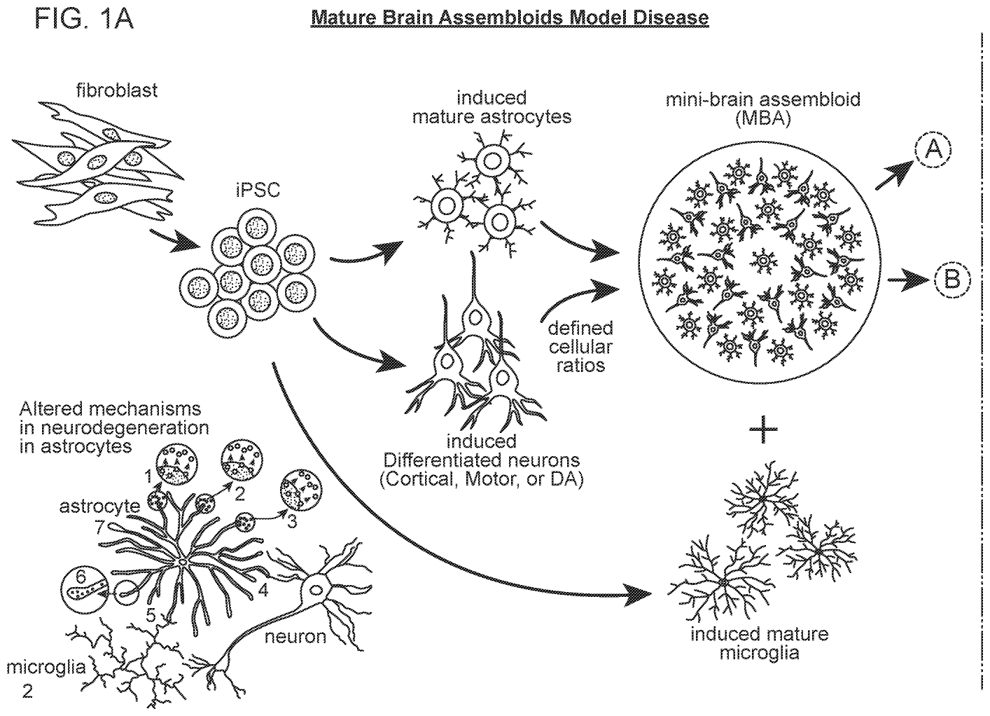

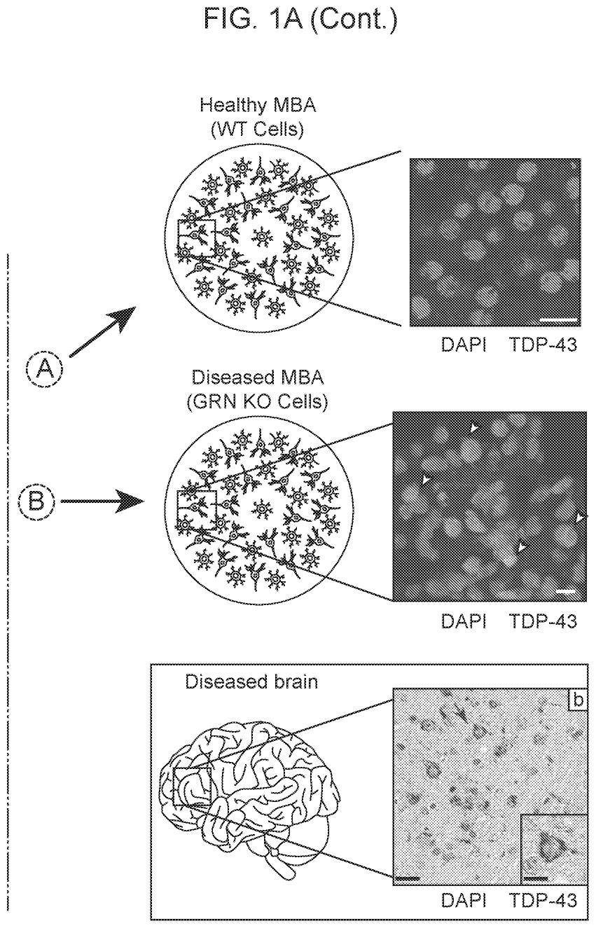

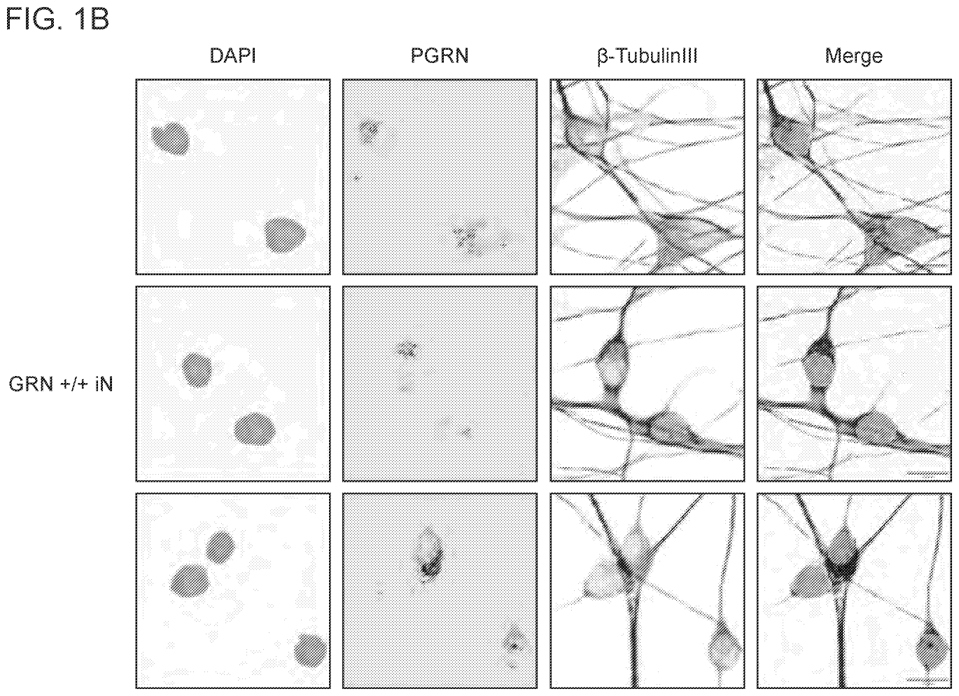

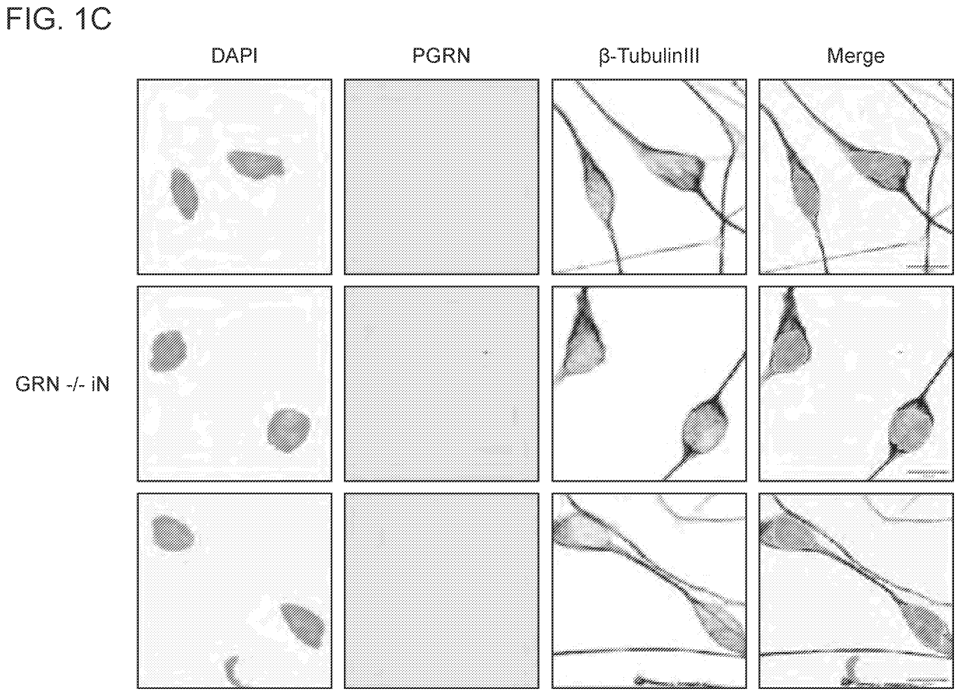

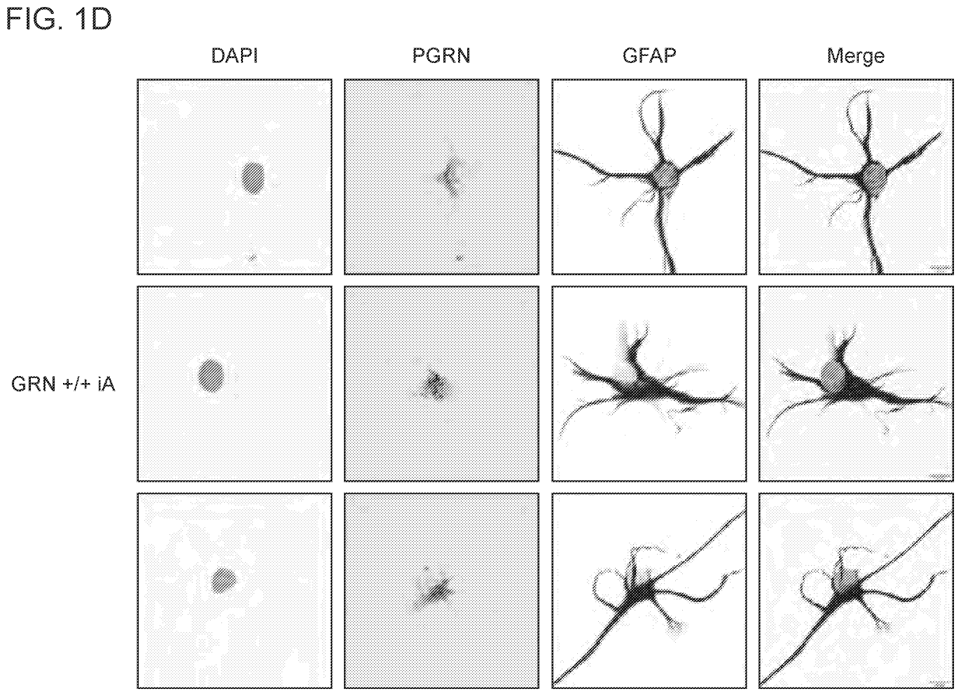

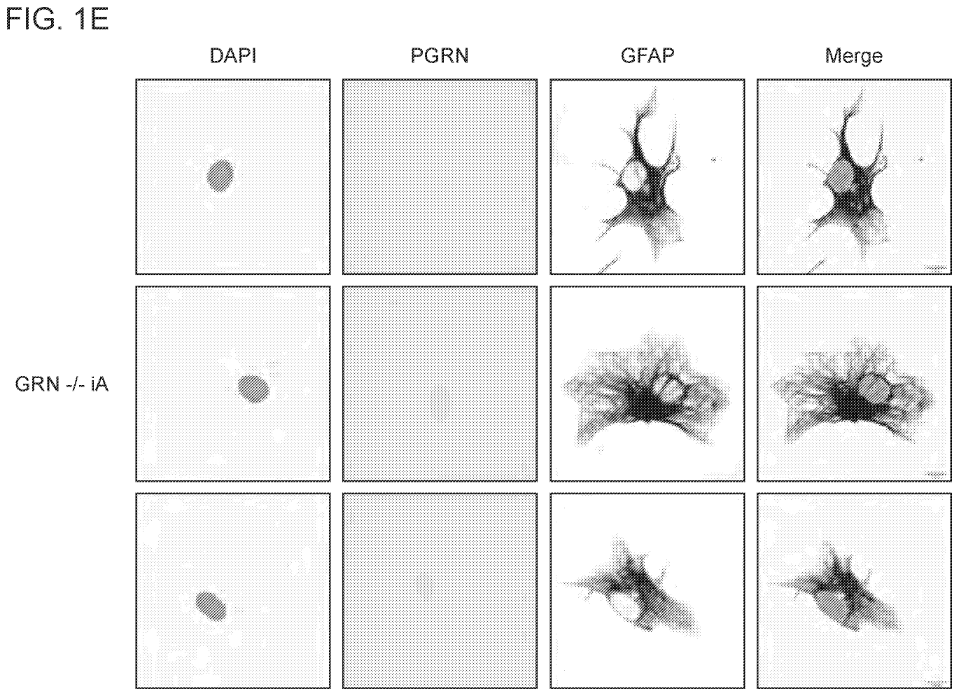

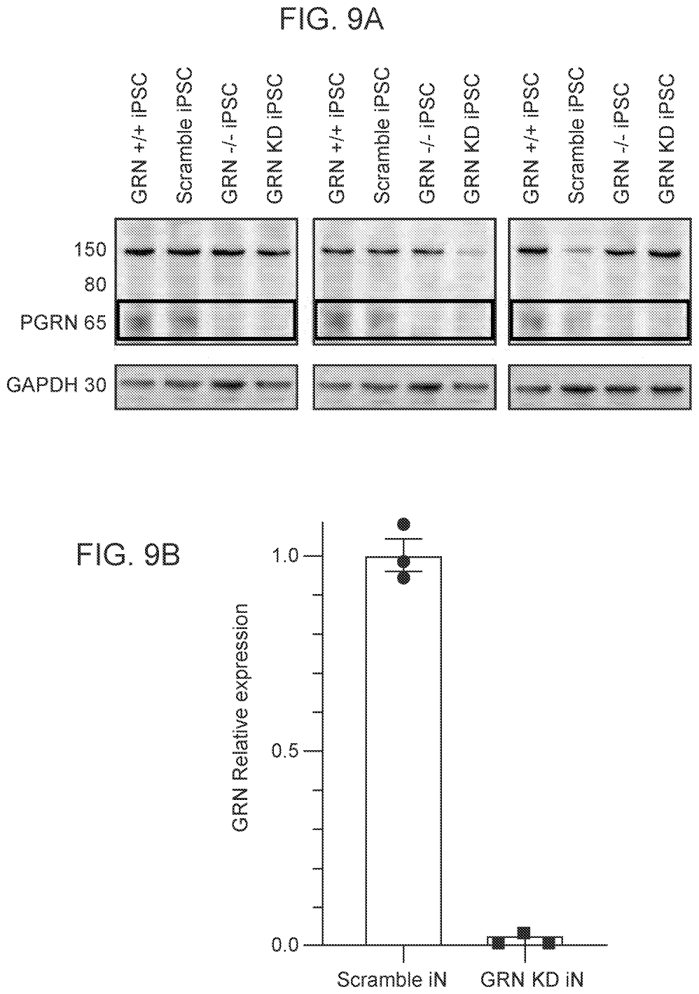

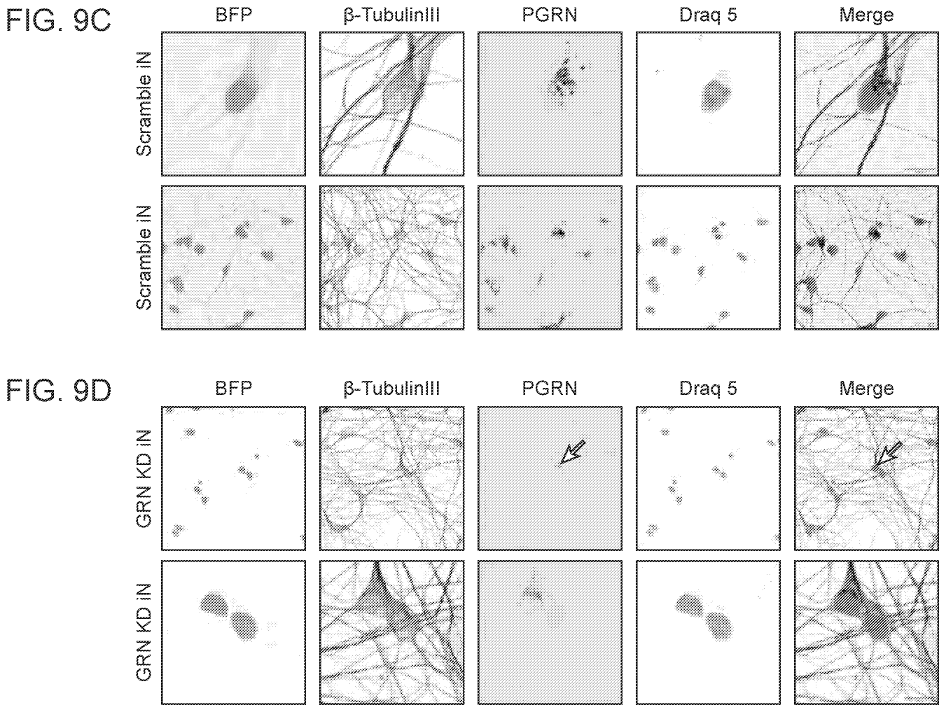

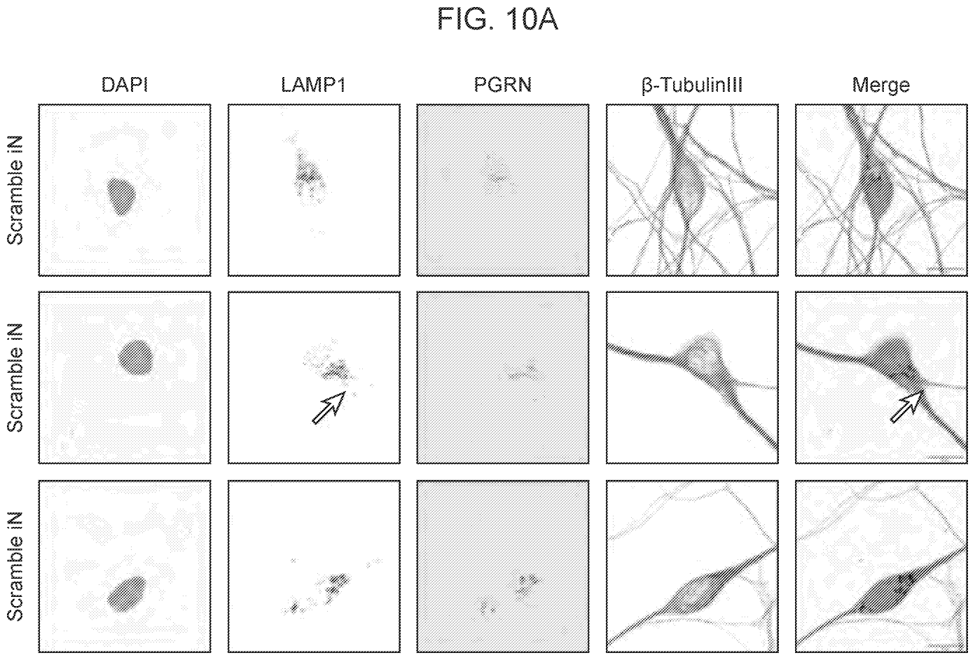

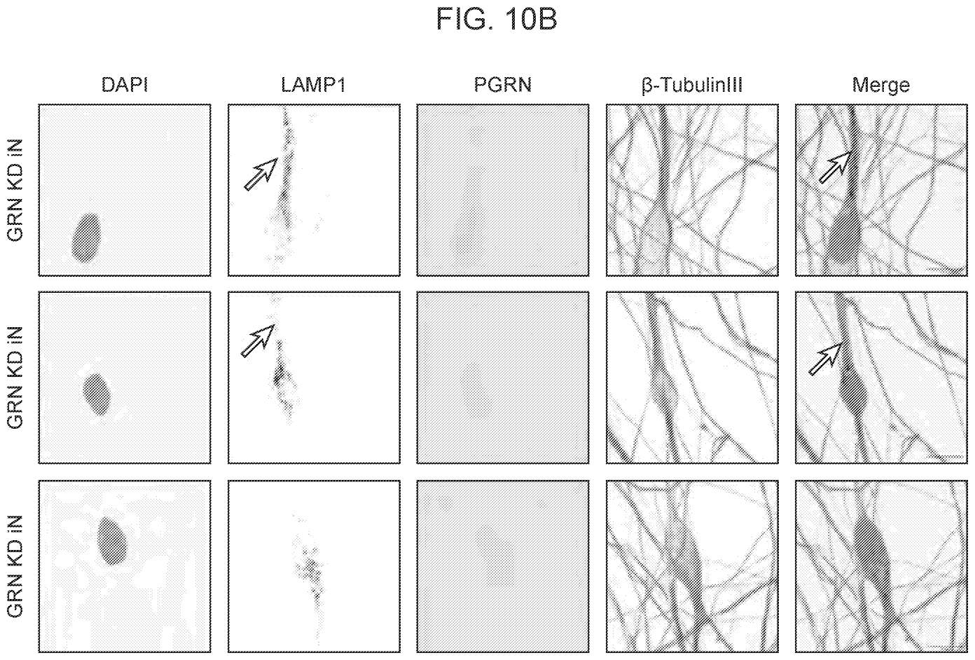

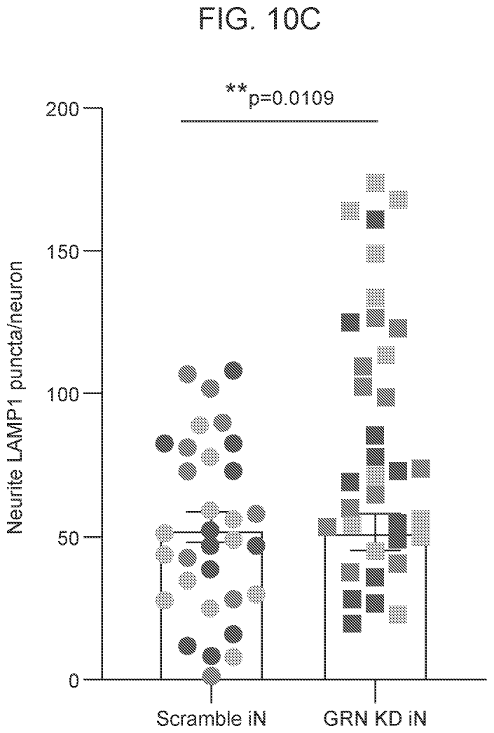

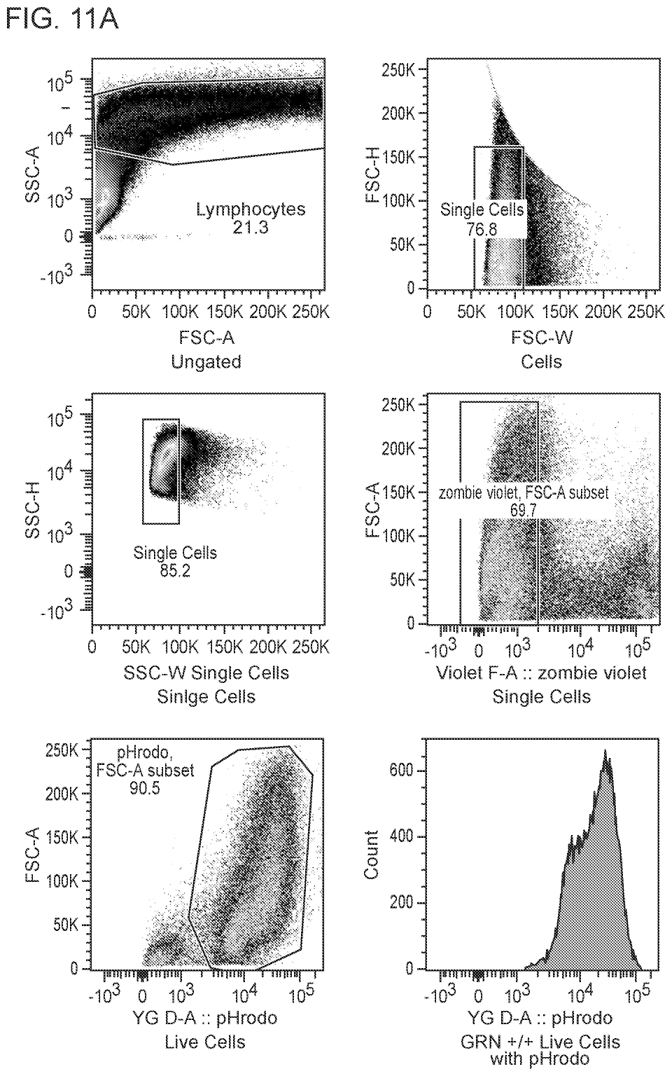

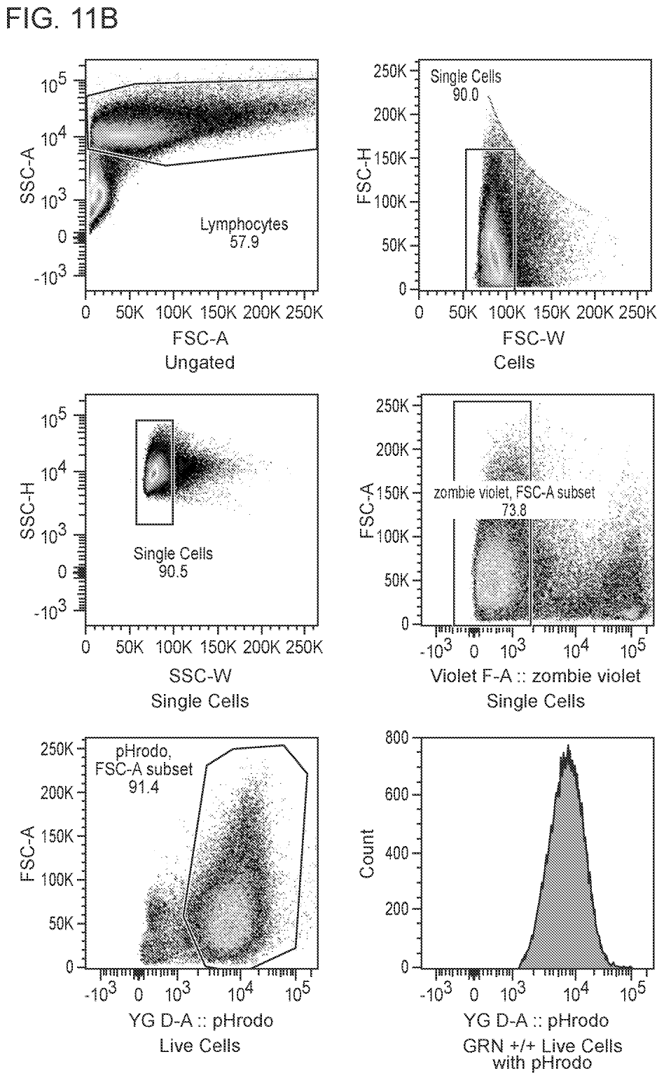

We adapted our assembled approach to 3D neuronal-glial cultures to study GRN deficiency. GRN KO iPSCs were generated by Michael Ward (FIG. 1A). These iPSCs were WTC11 background and one line was further engineered with the doxycycline inducible NGN2 cassette^{45;46}). To confirm loss of GRN in KO iPSC and GRN KO NGN2 iPSCs, we performed Western Blotting. GRN was nearly absent in GRN KO iPSC lines when normalized to GAPDH (FIG. 9). Next, we examined the localization of GRN protein in iPSC-derived neurons. GRN KO iN were induced along with isogenic WT iN controls and cultured for 2 weeks using conventional 2D culture approaches^{46;47;48}). At 2 weeks, cultures were stained for GRN protein which is expressed by multiple CNS cell types 39. In WT cells, PGRN staining can be detected in neuronal cell bodies (FIG. 1B). In contrast all GRN KO neurons were devoid of PGRN puncta when assessed by immunostaining (FIG. 1C). We repeated this staining on isogenic WTC11 GRN KO and WT lines differentiated into astrocytes. Nine months old iPSC-derived astrocytes (iAstrocytes) were similarly immunostained for PGRN. PGRN staining was visible in astrocyte cell bodies (FIG. 1D). In contrast PGRN staining was absent in GRN KO iAstrocytes (FIG. 1E). These results indicate that PGRN expression is efficiently curtailed in the GRN KO iPSC-derived neurons and astrocytes.

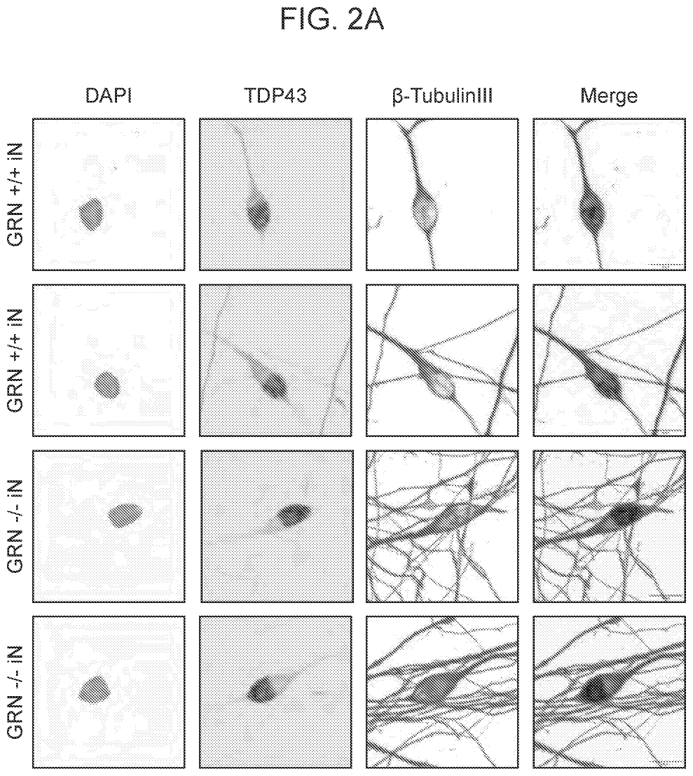

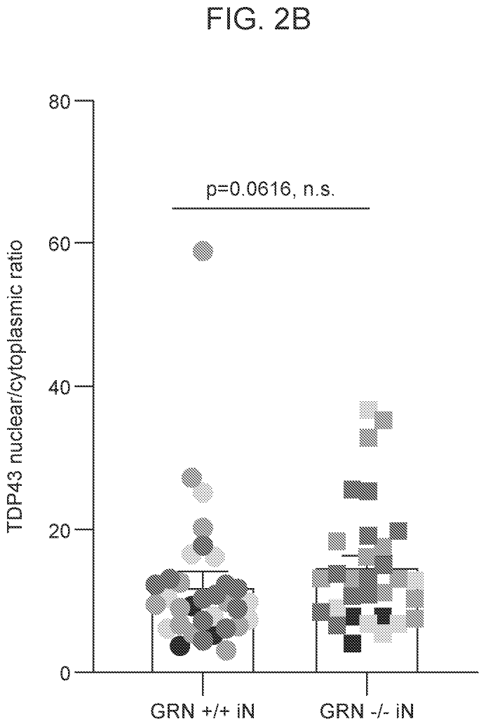

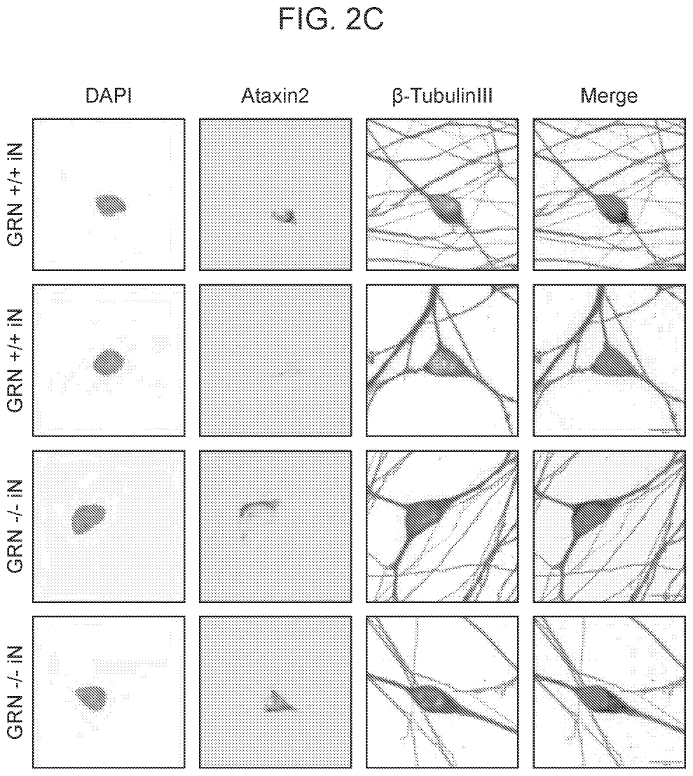

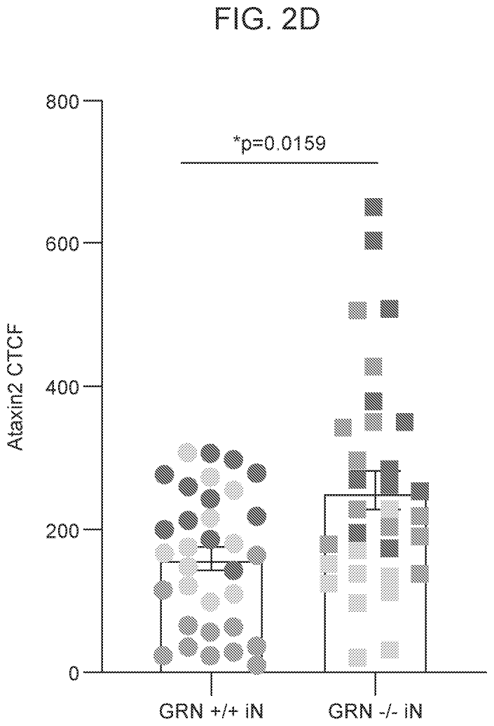

To assess phenotypes associated with PGRN loss we initially characterized nuclear and extranuclear TDP-43 in cultures of GRN KO and WT iN neurons. Depletion of nuclear TDP-43 is one of the key characteristics in postmortem brain tissue from patients with FTLD-TDP^{26;49} including FTLD associated with GRN mutations³¹. Importantly, nuclear depletion of TDP-43 is challenging to reproduce in many mouse³³ and iPSC models⁵⁰. Conventional 2D cultures of iN GRN KO and isogenic WT control neurons were immunostained for TDP-43 after 2 weeks in vitro. Nuclear TDP-43 did not change significantly in GRN KO iN compared with isogenic WT iN (FIGS. 2A, 2B), indicating that isolated neurons do not display strong cell autonomous TDP-43 phenotypes with GRN loss.

To determine if other disease associated changes could be detected we immunostained for Ataxin2 (FIG. 2C). Ataxin2 is an RNA-binding protein that plays important role in stress granule formation as well as interaction with processing bodies^{51,52}. Ataxin2 also contributes towards neurodegenerative disease pathology through stress granule induction and has been reported to recruit insoluble TDP43^{53,54}. Interestingly, lowering Ataxin2 level in a mouse ALS model has been reported to attenuate TDP43 pathology 55. There was no obvious increase in the formation of Ataxin2 granules, a characteristic feature of stress granule formation, in GRN KO compared to WT cultures (FIG. 2C). Interestingly there was a significant increase in the overall intensity of Ataxin2 staining in the cell bodies of a subset of GRN KO neurons (FIG. 2D). These results indicate that loss of GRN in neurons does lead to alterations in Ataxin2 expression and/or localization in a cell autonomous manner after two weeks of pure neuronal culture in 2D; yet, does not lead to overt Ataxin stress granule formation.

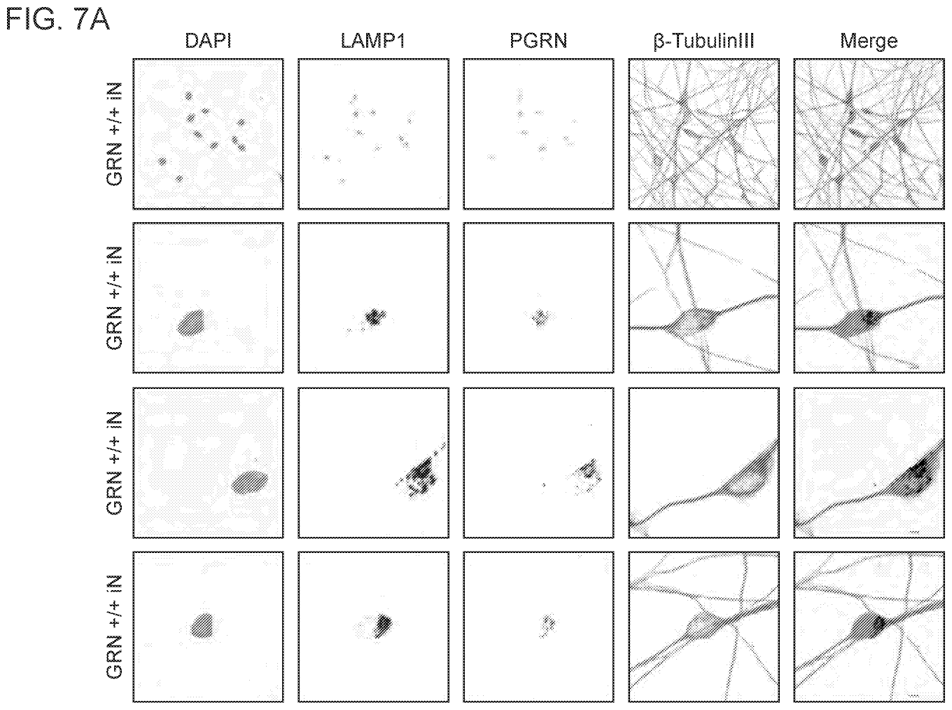

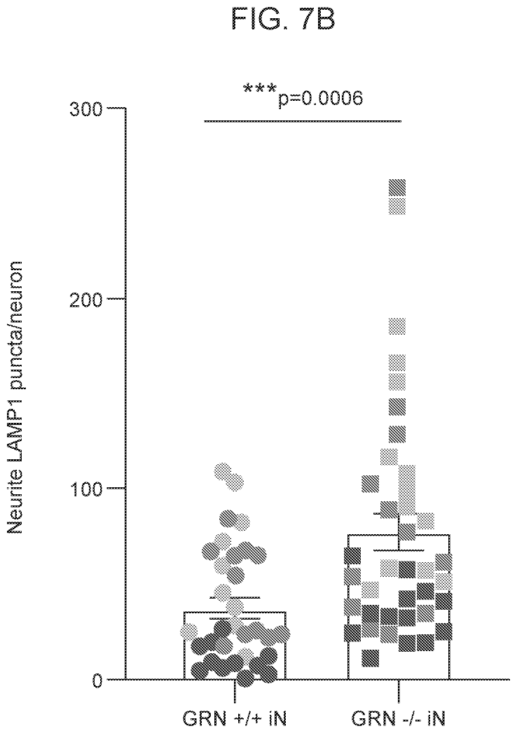

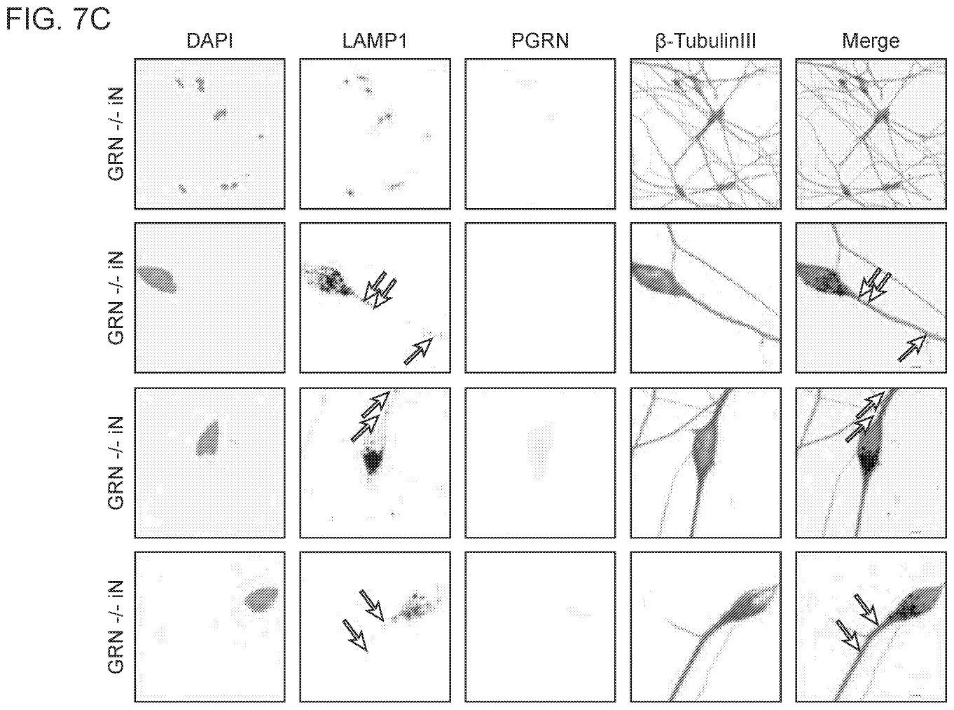

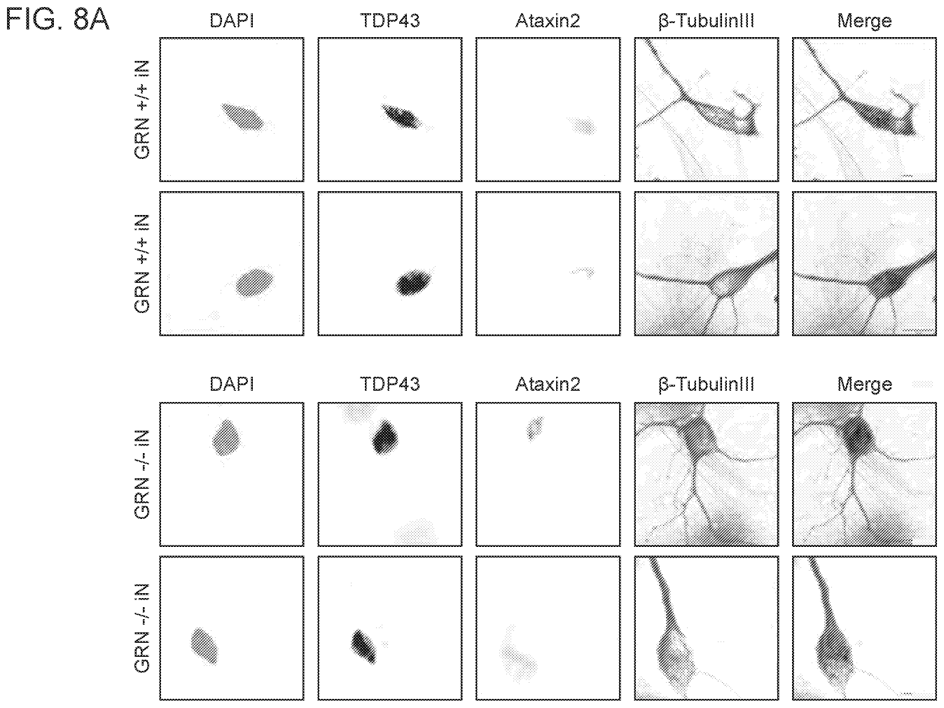

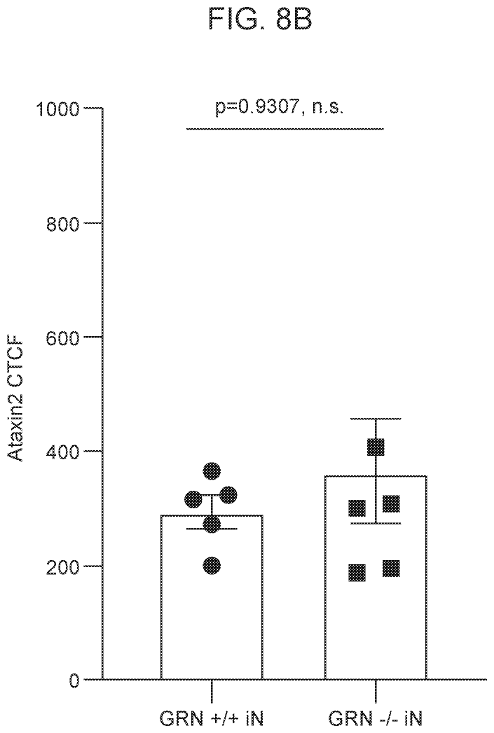

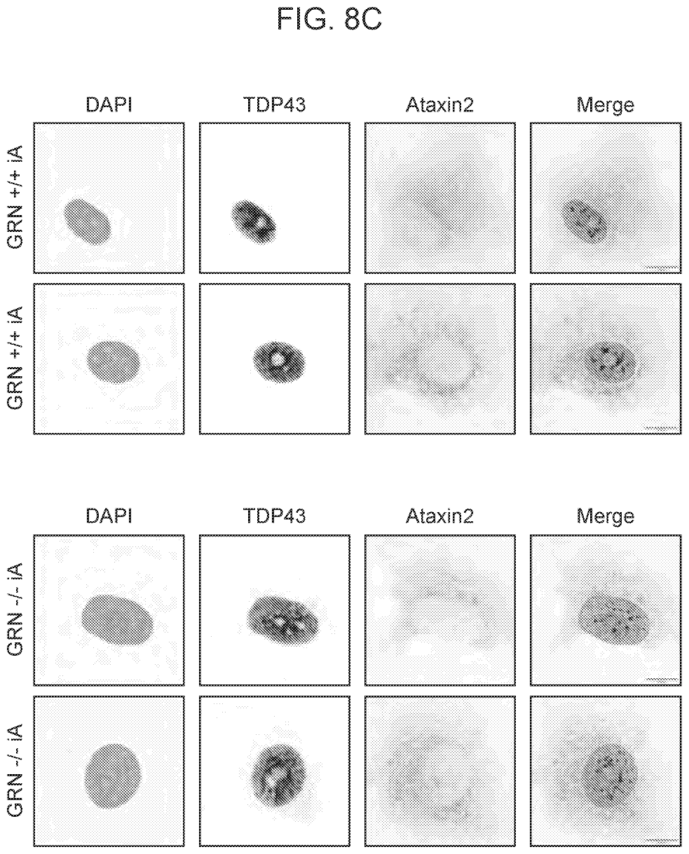

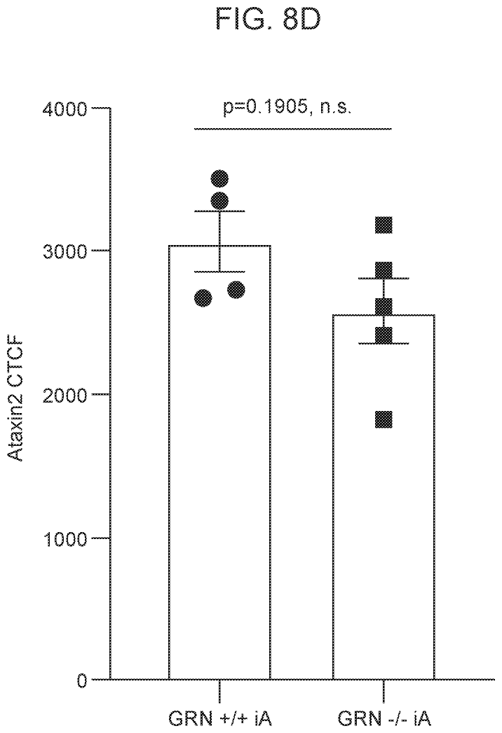

Finally, we investigated lysosomal changes in GRN KO iN by immunostaining for the lysosomal protein LAMP1. PGRN function in lysosomes and the granulin peptides derived from the pro-protein have been described as having important lysosomal regulatory functions^{56;57;58}. In WT isogenic controls LAMP1 is clustered in neuronal cell bodies similar to previous descriptions (refs, FIG. 7A). In contrast, in GRN KO iNs, LAMP1 staining was measurably increased along neurites, indicative of disruption of stable lysosomal networks (FIGS. 7B, 7C). Thus, loss of GRN in neurons does lead to subtle cell autonomous lysosomal phenotypes.

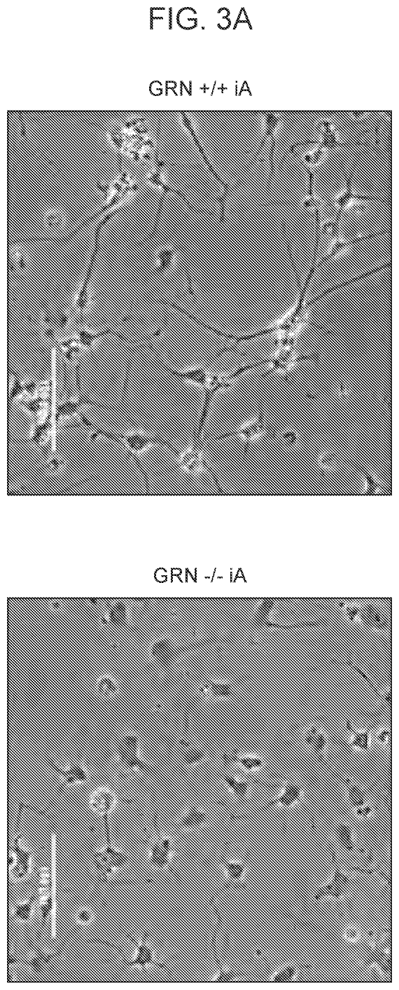

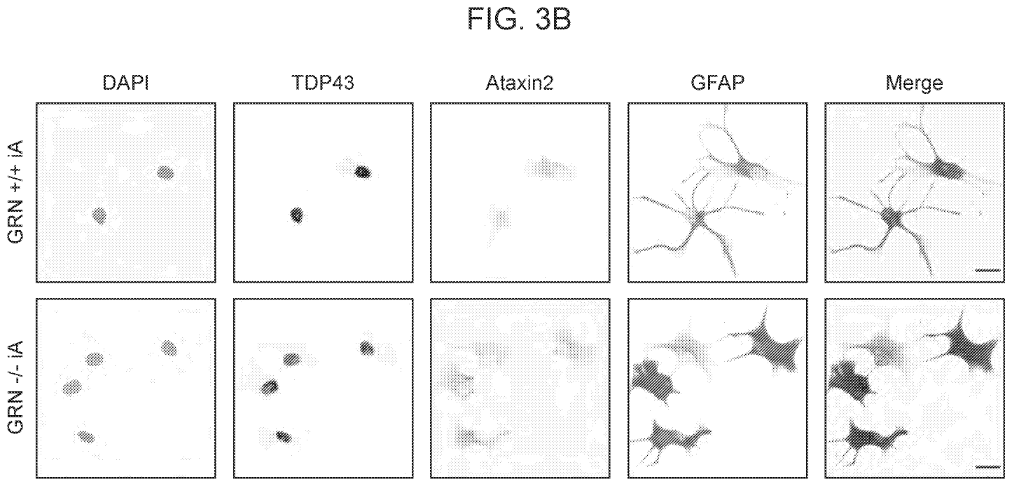

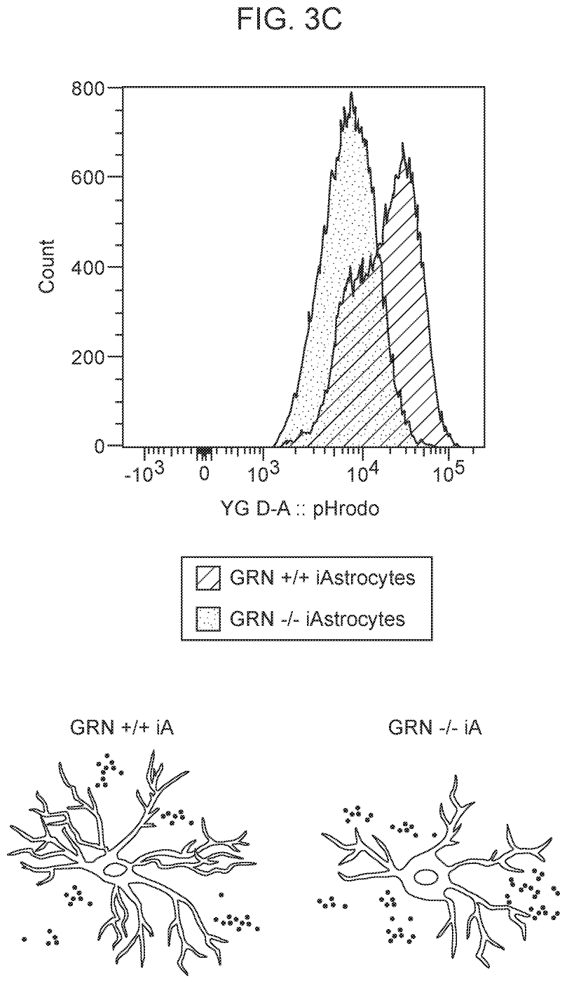

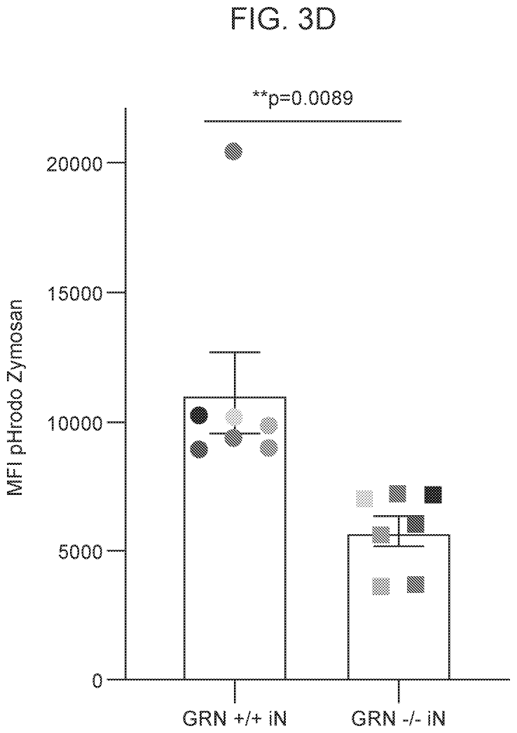

We next investigated whether GRN KO astrocytes have clear phenotypic differences when compared to isogenic controls. We utilized the mature GRN KO and WT iAstrocytes using a well-established protocol⁵⁹ (FIG. 3A). 9-month-old WT or GRN KO iAstrocytes were plated in 2D cultures and supplemented with CNTF and BMP4 for 2 weeks (FIG. 3b). We observed robust TDP43 nuclear localization in both WT and GRN KO iPSC-derived astrocytes (FIG. 3B). Similarly, Ataxin was primarily diffuse with few puncta in both WT and GRN KO iAstrocytes (FIG. 3B). iPSC derived astrocytes can recapitulate many aspects of neurodegenerative disease phenotype^{60,61}. To more carefully characterize the GRN KO human astrocytes, we examined if GRN KO iAstrocytes have functional or morphological changes. GFAP was readily detectable in both WT iAstrocytes and isogenic GRN KO iAstrocytes (FIG. 3B), however, GRN KO iAstrocytes displayed a more hypertrophic morphology similar to that described for reactive astrocytes^{62,63} (FIG. 3B). To determine if GRN KO astrocytes display physiological differences, we used zymosan conjugated pHrodo dye to investigate phagocytosis. Measuring dye uptake with FACS, we found that phagocytosis is impaired in GRN KO iAstrocytes compared to isogenic controls (FIGS. 3C, 3D). Interestingly, astrocytes are known to become reactive during brain injury as well as in many neurodegenerative diseases^{64;63} and decreased phagocytosis is consistent with previous functional descriptions of AI-like reactive astrocytes^{64 63} These data suggest GRN KO iAstrocytes have diminished capacity to clear debris in vitro and, perhaps, in vivo under FTD disease states.

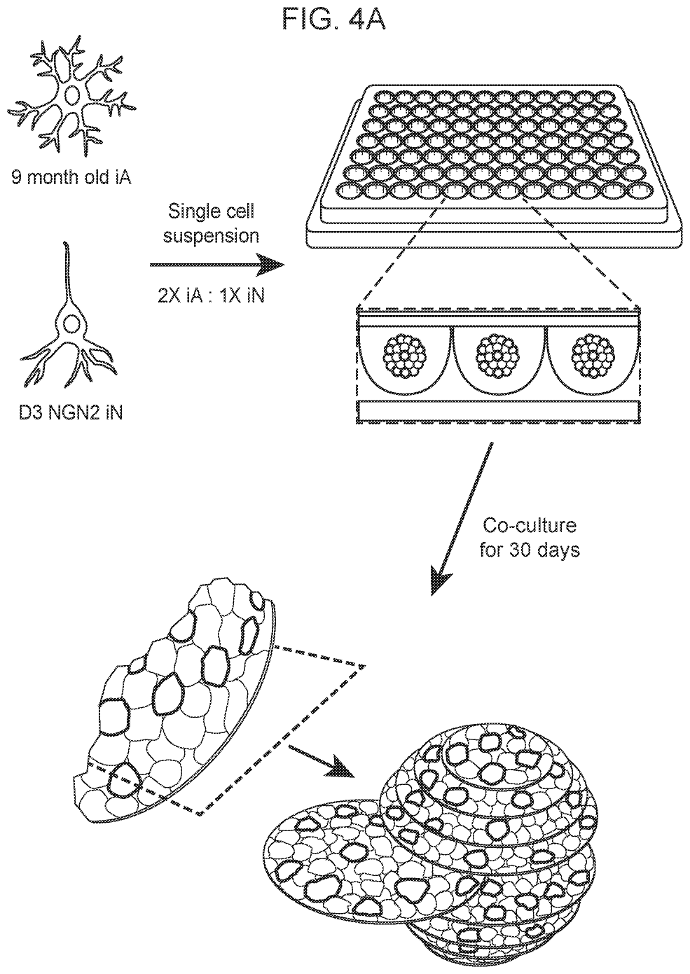

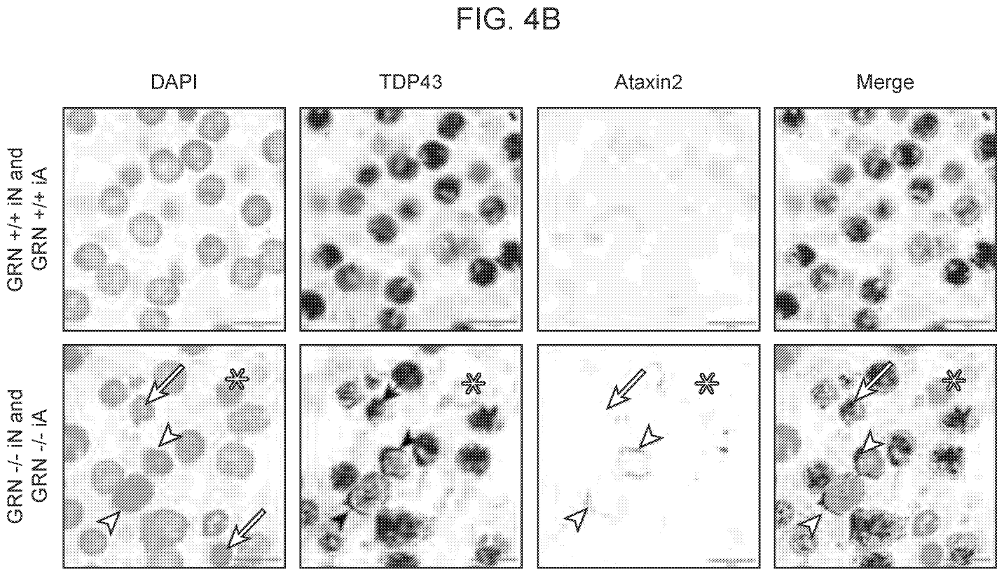

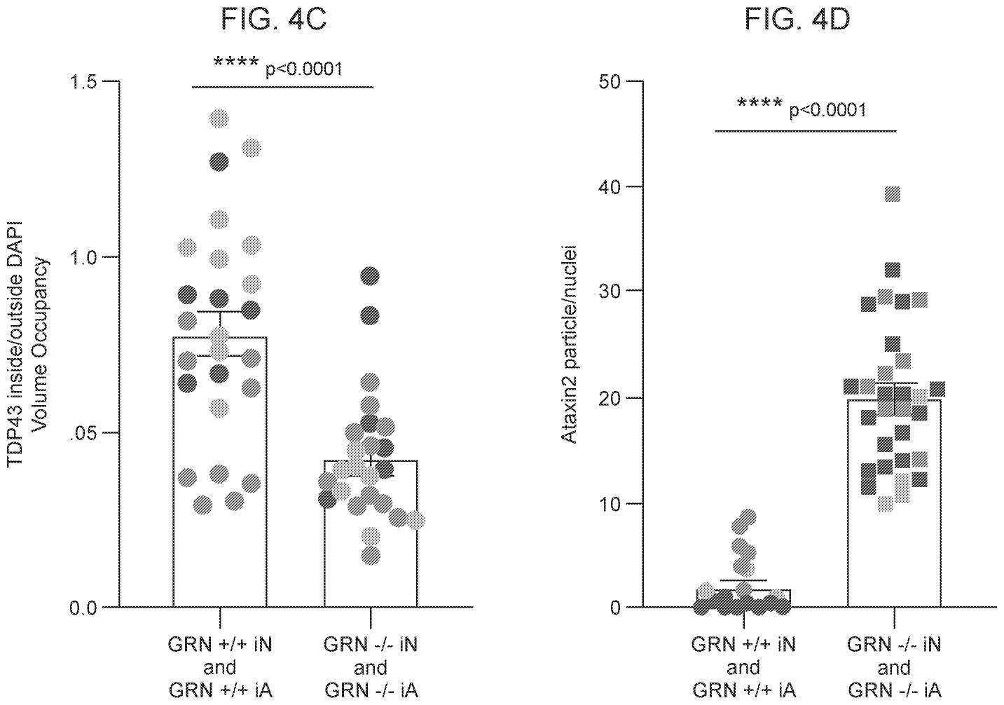

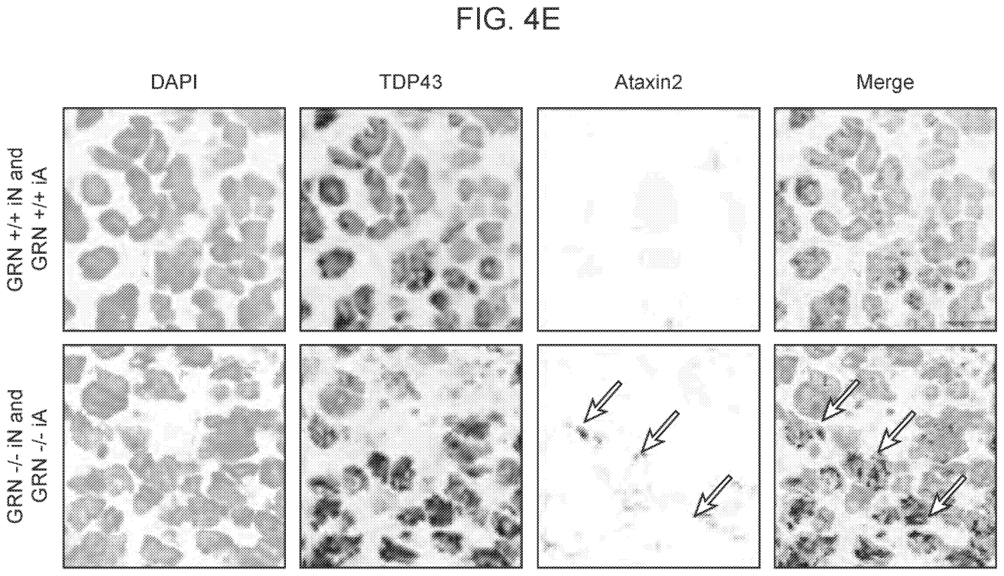

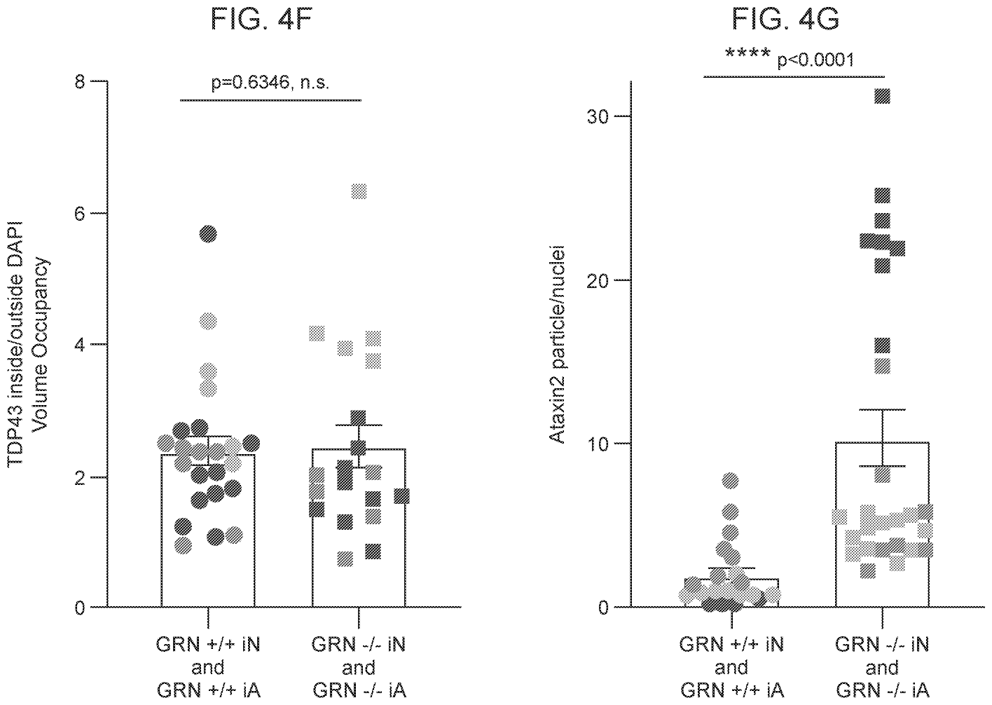

Combined, these data indicate that loss of GRN affects both human neurons and astrocytes leading to cell autonomous phenotypes. To better understand how the interplay between neurons and astrocytes in FTD leads to disease phenotypes, we developed a modification of our published 3D human assembled organoid system consisting of defined numbers and ratios of neurons and astrocytes⁶⁵ (FIG. 4A). This approach is advantageous because, first, the 3D assembled cultures contain only mature neurons and astrocytes. Second, we can control the exact ratio of cell types to better model the ratio reported in human cortex⁶⁶. Third, this approach is highly reproducible generating assembled organoids of uniform size and cellular ratios over many experimental runs. We previously reported that a similar 3D assembled approach enhances diseases phenotypes²⁸ so we decided to culture our human neurons and astrocytes using this modified approach (see Methods). First, we examined the nuclear associated protein TDP43 in isogenic WT 3D assembled organoids consisting of 2:1 ratio of astrocytes to neurons. We found that TDP43 remained nuclear over the course of four weeks in vitro in the 3D cultures (FIG. 4B). Next, we analyzed TDP43 in GRN KO cultures using the same 2:1 ratio of astrocytes to neurons. At four weeks in culture, we found a clear phenotypic difference in TDP43 staining. TDP43 showed a range of depletion phenotypes from partially nuclear depleted (FIG. 4B, arrow) to completely depleted from the nucleus (FIG. 4B asterisk). Significantly, we observed TDP43 aggregating just outside of many nuclei (FIG. 4B arrow head). Importantly, this type of TDP43 nuclear depletion and extra-nuclear aggregation has been reported in human postmortem brain with FTD and ALS^{31,36,67,27}. Previous work in mouse retina found a decreased nuclear to cytoplasmic ratio of TDP43 in GRN KOs compared to WT controls³¹. We adopted a similar approach to quantify the nuclear to extranuclear ratio of per section in mAssembloids. Through the quantification of volume occupancy (see methods), we were able to quantify both the signal of nuclear and extranuclear TDP43 signal within each mAssembloid section. Of 3 biological repeats, consisting of over 30 individual mAssembloids, we found that GRN KO mAssembloids have a lower ratio of intranuclear signal to extra-nuclear TDP43 signal compared to isogenic WT controls (FIG. 4C). These data indicate that the 3D assembled organoids show clear phenotypic differences between GRN KO and WT even when analyzed on a whole-organoid section level.

These data demonstrate that by 4 weeks in vitro GRN KO assembled organoids display clear phenotypes associated with human neurodegeneration. We next wanted to determine if other previously implicated neurodegenerative pathways and mechanisms might be occurring in this system. We again focused on stress granules because the formation of stress granules has been implicated as a possible early step in protein aggregation such as occurs with TDP43⁵³. We examined Ataxin2 as a well-known stress granule marker. As expected for healthy WT assembled organoids, Ataxin2 punctate staining was largely absent in the WT 3D cultures. This suggests that the WT mAssembloids have low levels of cellular stress⁶⁸. In contrast, GRN KO assembled organoids showed clear Ataxin2 punctate staining at 4 weeks (FIGS. 4B, 4D).

We next asked if Ataxin2 stress granule formation may be an early event in the GRN KO 3D cultures. We generated 3D cultures of WT and KO organoids and cultured them for 2 weeks and then immunostained for Ataxin2 and TDP43. TDP43 showed no significant difference in GRN and WT 3D cultures at 2 weeks indicating that the TDP43 phenotype is a late-stage phenotype seen in vitro (FIGS. 4E, 4F). In contrast, clear increases in Ataxin2 staining were seen in the GRN KO 3D cultures at 2 weeks compared to isogenic controls (FIGS. 4E, 4G). This indicates that stress granule formation may be an early step in expressed pathological phenotypes in our 3D culture system^{13,69}.

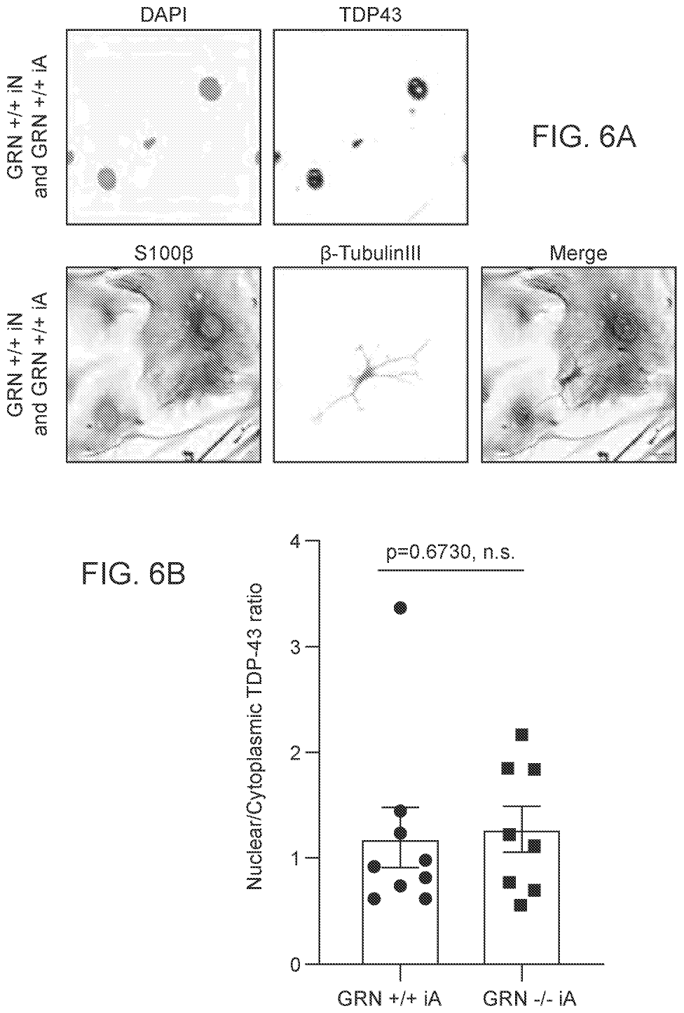

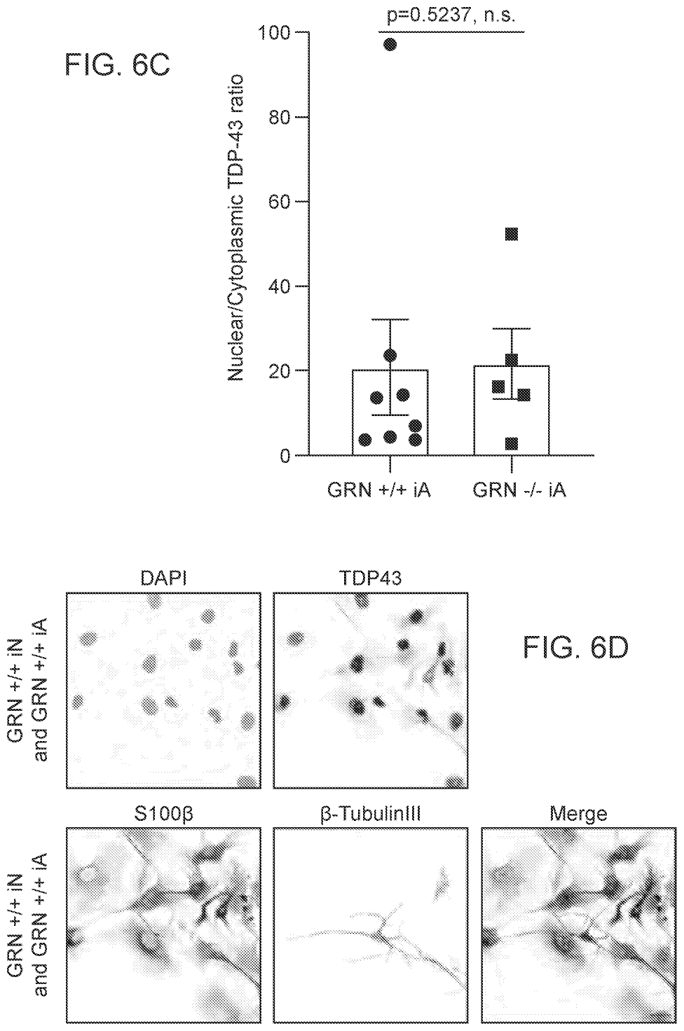

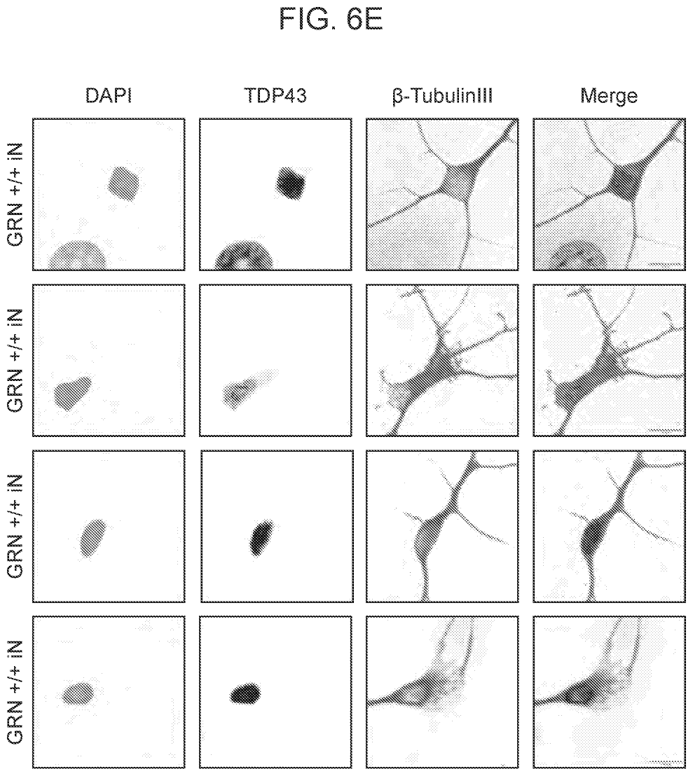

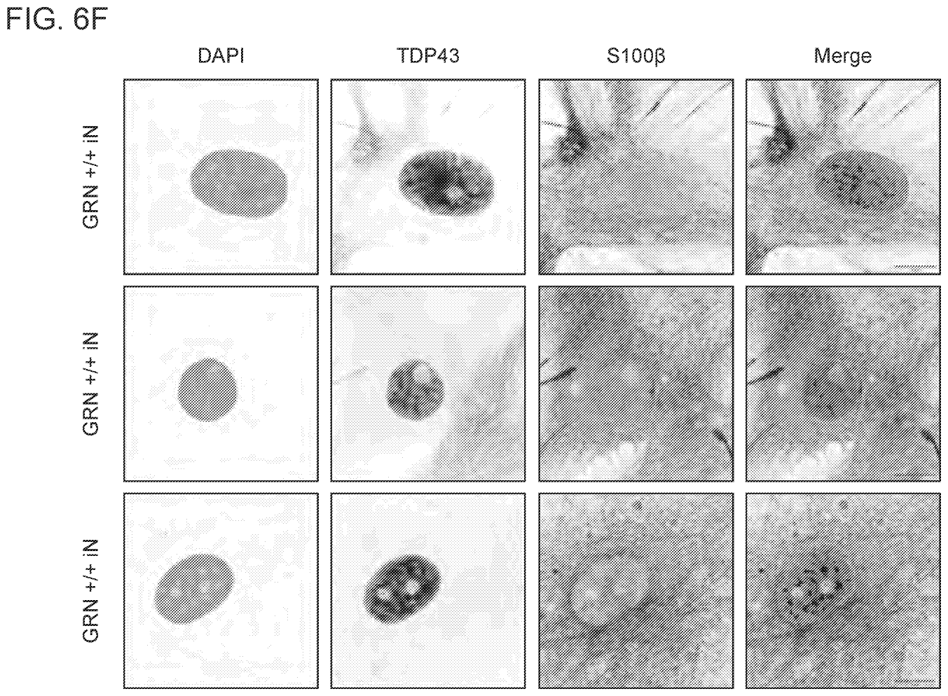

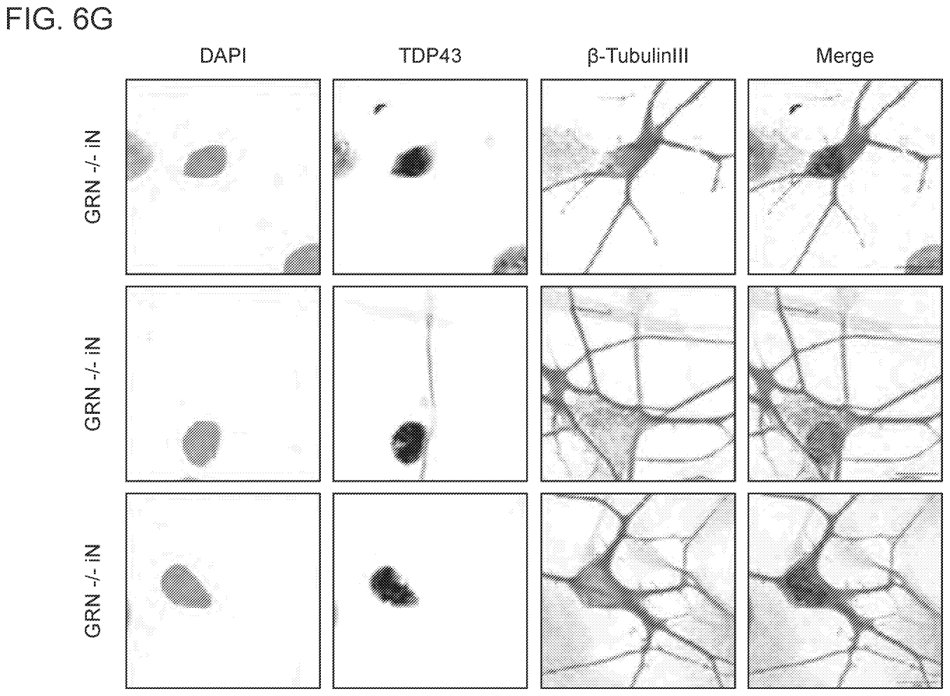

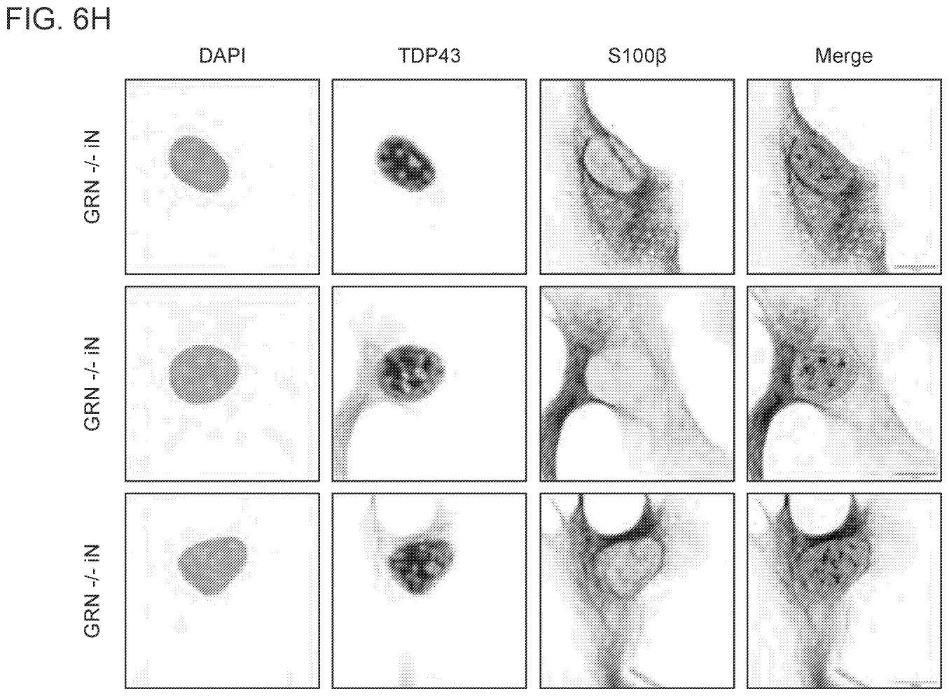

To test if similar phenotypes could be detected in 2D culture, we also co-cultured the iNeurons and iAstrocytes in 2D for 14-21 days (FIG. 6). We did not observe any non-nuclear TDP43 pathology (S3a-d) nor examples of stress-granule Ataxin2 puncta in the GRN KO neuron and astrocyte co-cultures (S1a-h S3a-d). Ataxin2 total fluorescence was not significantly changed in 2D co-culture between WT and GRN KO neurons (p=0.52) and between WT and GRN KO astrocytes (p=0.17). We could not analyze 2D cultures at 30 days because, unlike 3D mAssembloids, neurons showed significant apoptosis in both WT and GRN KO culture conditions (data not shown). This result suggests that the 2D culture technique is not sufficient to recapitulate neuron-glia interactions that lead to pathological phenotypes.

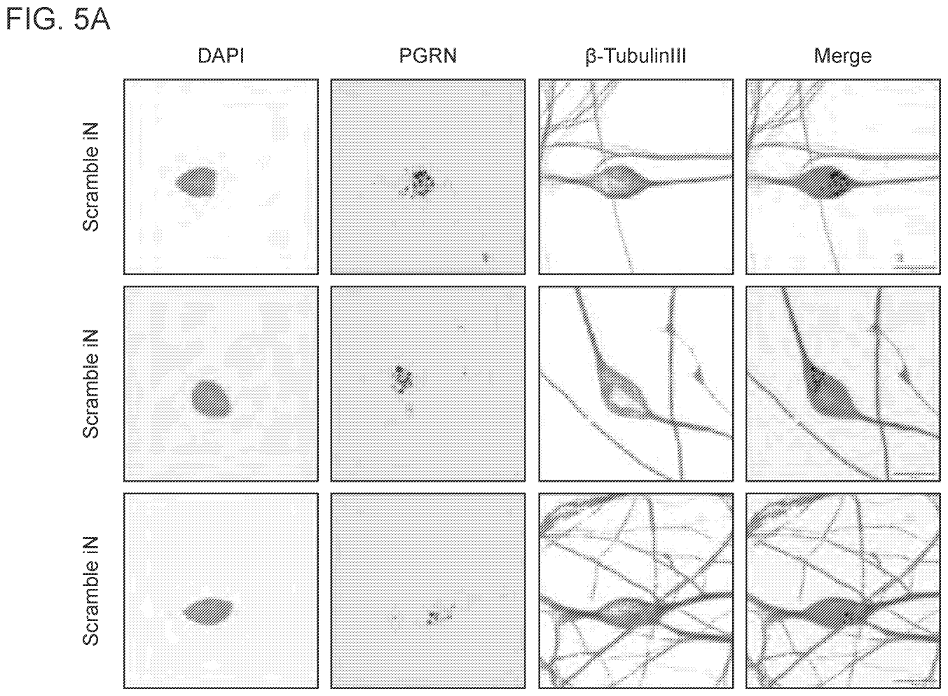

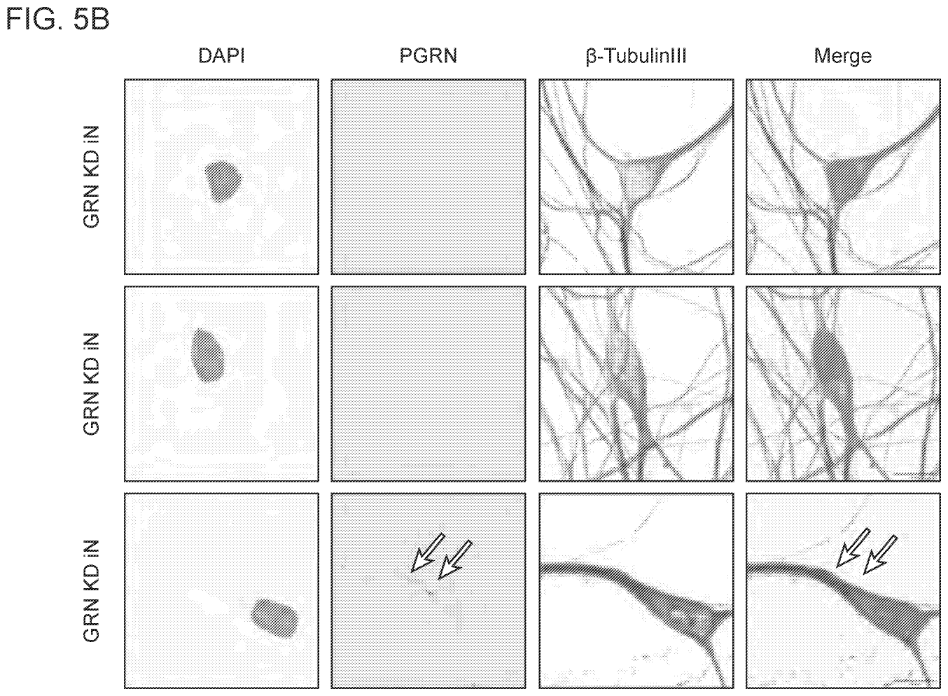

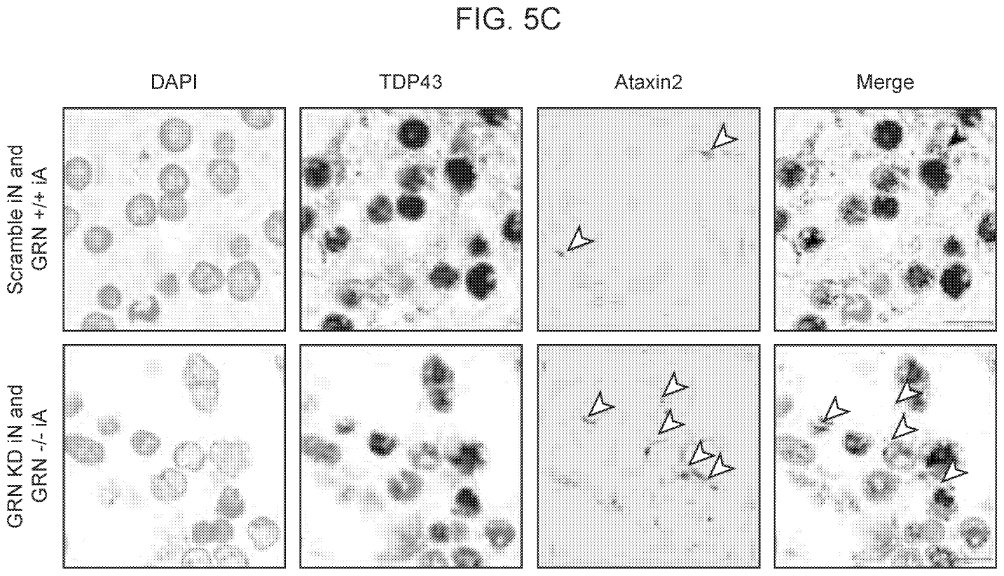

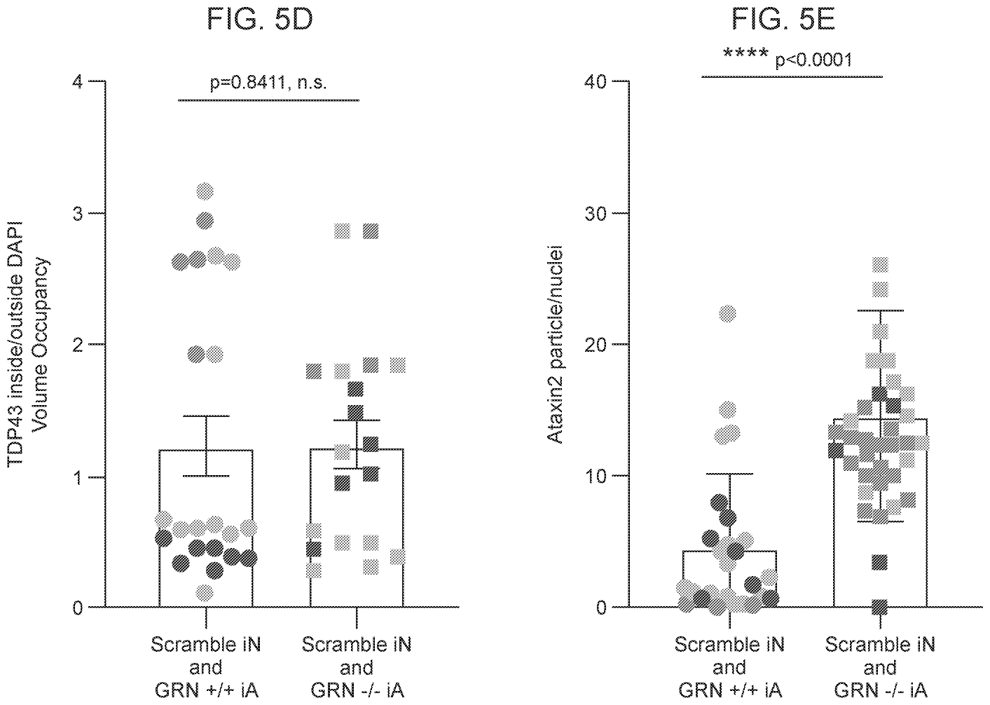

In order to further investigate the role of GRN loss, we generated GRN CRISPRi knockdown lines⁷⁰. Human FTD is caused by GRN haploinsufficiency³), and we wanted to examine if partial GRN loss recapitulates the TDP43 or Ataxin2 phenotypes in the 4-week time-course of these experiments. Using the WTC11 CRISPRi system⁷¹, we generated both scrambled guide RNAs and GRN guide RNAs to knocked down GRN in induced neurons (FIGS. 5A, 5B). Interestingly, this line showed strong knockdown of PGRN in iPSCs by Western Blot (FIG. 9A) but immunostaining of PGRN CRISPRi KD neurons showed occasional PGRN positive puncta in cell bodies (FIGS. 5B, 9C, approximately 1 in 40 neurons showed PGRN staining), indicating that the CRISPRi knockdown was not a complete loss of protein. To further characterize the development of pathological features in neurons with reduced, but not complete loss of PGRN, we cultured these neurons with GRN KO iAstrocytes for 4 weeks using the same mAssembloid approach described above (FIG. 4A). Interestingly, we did not observe strong loss of nuclear TDP43 in mAssembloids at 4-week and measured no significant change in the nuclear to extranuclear ratio of TDP43 compared to CRISPRi scrambled control iN and WT iAstrocyte containing mAssembloids (FIGS. 5C, 5D). However, we did detect a significant difference in Ataxin2 puncta (FIGS. 5C, 5E). These data suggest that stress granule formation and Ataxin2 pathology may be an early marker for disease progression and that partial loss of GRN in neurons is less severe than complete knockout in vitro (FIG. 5); consistent with described patient outcomes for homozygous vs heterozygous GRN mutations^{72;3;73;74;75}. Furthermore, these results indicate that there are neuronal cell-autonomous mechanisms occurring in the 3D assembled organoid as GRN KO astrocytes cultured with GRN KD neurons showed a rescue of TDP43 in GRN KD neurons when even low amounts of PGRN are present. The 1 in 40 neurons showing PGRN expression corresponds to approximately 167 neurons per 20,000 cell mAssembloid consisting of ⅓ neurons and ⅔ astrocytes. This suggests that even low-level expression of GRN in a subset of cells could mitigate GRN loss in FTD.

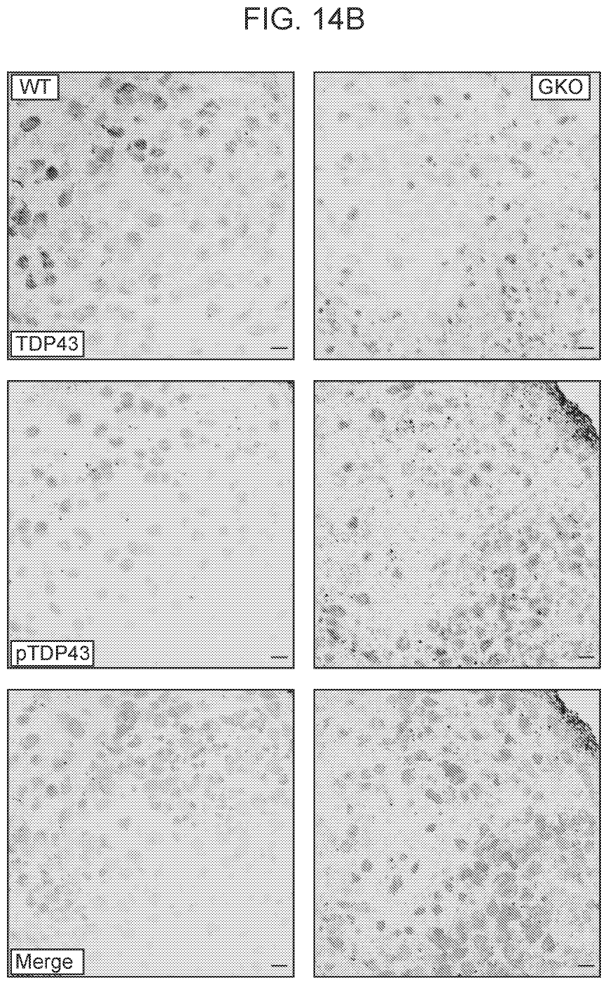

The assembloids showed increased phospho-TDP-43 in a GRN FTD disease model (FIGS. 14A-14B). FIG. 14A shows the quantification of phospho-TDP-43/TDP-43 ratio in 4 separate runs of assembloids from WT or isogenic GRN KO assembloids of neurons and astrocytes. FIG. 14B shows immunostaining of TDP-43, phospho-TDP-43 and DAPI in WT or isogenic GRN KO assembloids showing increased phospho-TDP-43 in GRN KO assembloids. Increased phospho-TDP-43 is a hallmark of human neurodegenerative disease but is not found in animal models and has not been reported in other stem cell models.

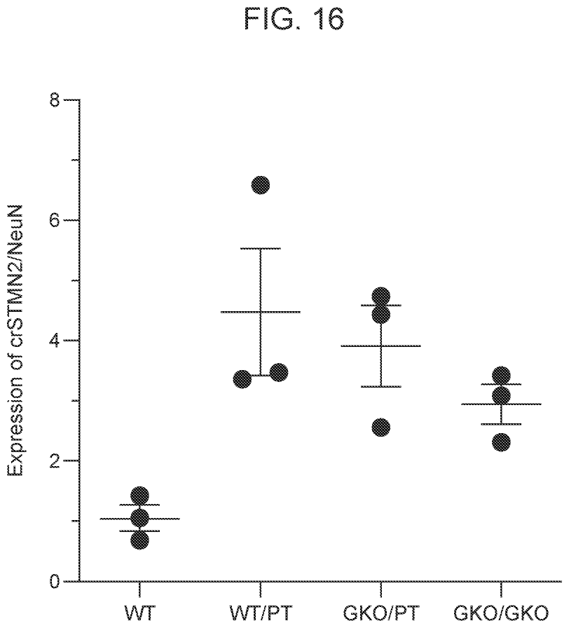

Patient-derived assembloids containing FTD/ALS mutation-bearing neurons showed cryptic STM2 expression indicating TDP-43 pathology (FIG. 16). All combinations of disease assembloids showed cryptic STM2 in assembloids.

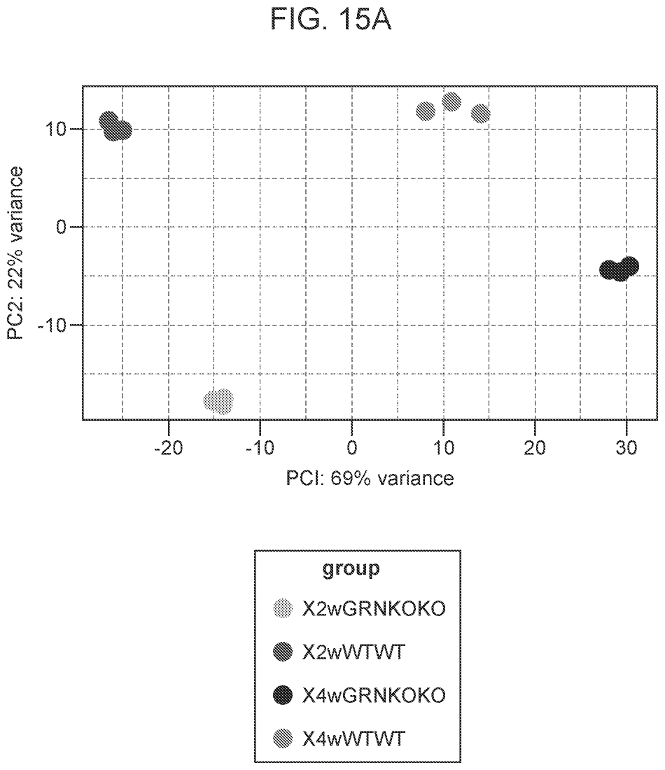

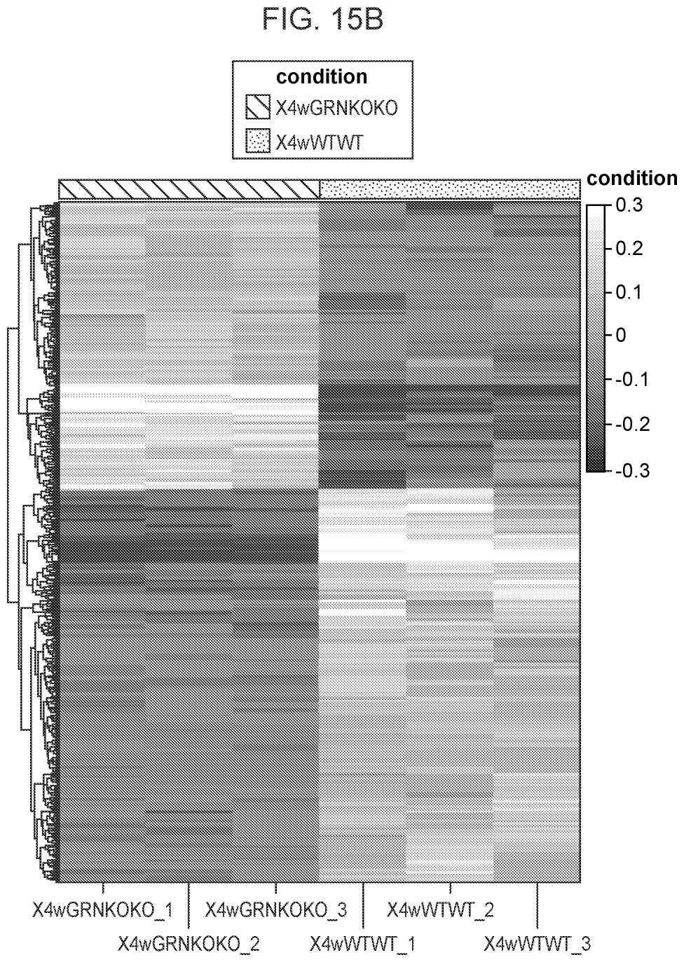

The assembloids showed high reproducibility among separate experiments (FIGS. 15A-15B). The coefficient of variation of gene expression in separate culture runs of GRN WT and KO assembloids show highly correlated gene expression among separate experiments indicative of high reproducibility (FIG. 15A). A heat map of gene expression showed similar direction of gene expression changes among three separate runs of isogenic WT and GRN KO assembloids (FIG. 15B), again indicating high reproducibility using the assembloid method.

### Discussion

### 3D Culture Approach

Here, we describe an in vitro neuron glia 3D model for investigating TDP43 proteinopathy in human iPSC-derived cell types. While iPSC induced neurons have provided numerous cell-autonomous biological insights into neurodegeneration, most models do not fully recapitulate TDP43 pathology similar to that seen in human brain. Our approach is unique in that we use a simply, reproducible, and straightforward engineered approach to model neurodegenerative diseases incorporating both neuron and glia cell types in a 3D model. Indeed, our 3D iPSC induced neuron and astrocyte co-culture with GRN loss strikingly recapitulated human specific TDP43 proteinopathy reported in postmortem brain. Importantly, these phenotypes are not seen in the same individually cultured iPSC induced neuron, astrocytes, or even neuron-astrocyte co-culture in 2D. The reason for this stark difference between 2D and 3D cultures is not entirely clear, but we have previously found that astrocytes in 3D culture are significantly more complex and resemble the highly complex morphology of human astrocytes in vivo²⁸. Furthermore, these results suggest that iPSC-derived astrocytes in 3D are not highly reactive by 4 weeks in vitro. This may further allow the clear differentiation of disease phenotypes using this culture approach and isogenic cells.

### Progression of Phenotypes

It is noteworthy that the TDP-43 phenotype is quantifiably increased by four weeks in vitro yet, at two weeks is not statistically significant by the method we used to assess pathology. Looking at potential early markers, we found that at two weeks in vitro, stress granules are upregulated in GRN KO mAssembloid prior to TDP43 pathology, and persist into the 4-week timepoint. The stress granule phenomenon was further confirmed with CRISPRi GRN Knock Down neurons. These neurons show a strong downregulation of GRN but still retain some GRN gene expression. Thus, it is not surprising that this line may yield a milder phenotype by four weeks in vitro. Taking advantage of this, we found clear Ataxin2 clustering yet no statistically significant change in TDP-43 using our imaging approach. This finding along with the early Ataxin phenotype seen at two weeks in the GRN KO 3D mAssembloids suggests that the early formation of stress granules may be an important cellular mechanism and this could have profound implication for potential therapeutic approaches⁵⁵. Lastly, our investigation of phagocytic activity assay in GRN KO astrocytes demonstrated significant phagocytic defects. This finding is consistent with a more reactive-like phenotype⁶⁴. This finding along with the clear necessity for GRN KO astrocytes in 3D cultures with GRN KO neurons suggests that human astrocytes have cell-autonomous effects with GRN loss that contribute to 3D mAssembloid pathology. Future experiments will explore the cell autonomous and non-autonomous phenotypes uncovered by this methodology and allow more complex mix-and-match experiments using combinations of GRN KO and GRN WT neurons and astrocytes.

### Malleability of Approach

This platform is very malleable to future cell type investigation in neurodegenerative diseases. Differentiation protocols based on induced expression of transcription factors are particularly useful for gene mutation led phenotype investigation, as they are rapid and yield large numbers of homogeneous cells. Our utilization of the WTC11 NGN2 line facilitated our ability to rapidly assess phenotypes in the mAssembloid platform^{47;46;48}). In addition to the Ngn2-driven generation of cortical glutamatergic neurons and the protocol for generation of astrocytes^{59,28} used here, induced expression of different transcription factors can yield other types of neurons, such as motor neurons ^{76;77} and inhibitory neurons⁷⁸. These can be easily added to the 3D mAssembloid in defined ratios and numbers. These mAssembloid culture are also amenable to the addition of iPSC-induced microglia like cells^{79,80,81,82,83,84} Microglia are increasingly implicated in numerous neurodegenerative diseases so a system incorporating them is a necessary next step for model disease. The ability to incorporate CRISPRi lines, further enhances the utility of this approach to future screening capability⁷⁰. These approaches, to be refined and improved using the basic platform described here, can yield new therapeutic targets for neurodegenerative disease. Despite their utility, iPSC-derived cells have limitations—in particular, they do not fully recapitulate all features of the complex human brain. Nor do they currently incorporate vascular networks, although this may be possible^{85,86,87,88,89,90,91,92}). Despite these limitations, this approach provides a simple straightforward method to model neurodegeneration that can be readily incorporated into laboratories and expanded to study basic cellular interactions and mechanisms of disease.

### Material and Methods

### Human Induced Pluripotent Stem Cell Line

Isogenic iPSC line WTC11 was generated in Gladstone Institute by Dr. Bruce R. Conklin (Miyaoka et al., 2014). More information regarding this cell line can be found online (GM25256, Coriell Institute for Medical Research). NGN2 inserted WTC11 have been generated from previous publication (PMC5639430). GRN KO iPSC and GRN KO NGN2 iPSC were engineered and provided by Dr. Michael E. Ward (NIH). iPSCs were maintained at 37° C. and 5% CO₂ in a humidified incubator. IPSCs were cultured in Essential 8 Medium (Gibco/Thermo Fisher Scientific; Cat. No. A1517001) on 6 well Cell Culture plates (Eppendorf; 0030720113) coated with Matrigel GFR Membrane Matrix (Corning; Cat. No. CB40230) diluted at final 10 μg/ml concentration in DMEM/F-12, GlutaMAX supplement (Gibco/Thermo Fisher Scientific; Cat. No. 10565018). Briefly, Essential 8 Medium was replaced every day. When 80-90% confluent, cells were passaged, which entailed the following: aspirating media, washing with DPBS, incubating with StemPro Accutase Cell Dissociation Reagent (Gibco/Thermo Fisher Scientific; Cat. No. A11105-01) at 37° C. for 7 minutes, diluting Accutase 1:1 in DMEM, collecting in conical tubes (GENESEE Scientific Corporation, 28-103) centrifuging at 300 g for 1.5 minutes, aspirating supernatant, resuspending in Essential 8 Medium supplemented with 10 nM Y-27632 dihydrochloride ROCK inhibitor (Tocris; Cat. No. 125410), counting, and plating onto Matrigel-coated plates at desired numbers.

### GRN Knock Down and Scramble CRISPRi iPS Cell Line Generation

CRISPRi iPSC line was provided by Dr. Martin Kampmann (IND, UCSF) (Tian et al., 2019). GRN single guide (GRN_-_42422695.23-P1P2 GTGCCCAAGGACCGCGGAGT (SEQ ID NO:1) pLG15-1 MK) and no-known target scramble (negative_control-10016 GGACTAAGCGCAAGCACCTA (SEQ ID NO:2) pLG15-1 MK) options were used from previous publication (Horlbeck et al., 2016). HEK293 cells were transfected with sgRNA transfer plasmids and third generation lentiviral packaging mix using Mirus TransIT Lenti transfection reagent (MIR6600) following the manufacturer's protocol. Lentiviral supernatant was then concentrated using Allstem Lentivirus Concentration Solution (LV810A-1) following the manufacturer's protocol. 24 hours after infecting the CRISPRi iPSCs, cells were further selected with 0.75 μg/mL puromycin. To ensure selecting stable integration of GRN Knock Down and scramble single guides, cells were passaged 4 more generations with E8 media supplemented with 0.75 μg/mL puromycin.

### Induction of Neurons

NGN2 inserted iPSC (WT and GRN KO) line were engineered and shared by Michael E. Ward (NIH)^{46,47}. There were two steps for this induction, pre-differentiation and maturation. For pre-differentiation, iPSCs were cultured with Essential 8 medium (Thermo Fisher Scientific A1517001) on Matrigel coated cell culture plate. When 70%-80% confluent, cells were dissociated with accutase for 7 mins. iPSCs at a density of 300 k cells/well in six-well Matrigel coated cell culture plates again with pre-differentiation media, DMEM/F12 with HEPES (Gibco 11330032), Non-Essential Amino Acids NEAA (Gibco, 11140050), GlutaMAX (Gibco, 35050061), N2 supplement (Gibco, 17502048), supplemented with ROCK Inhibitor Y-27632 (R&D System, 125410) at 10 μM concentration, as well as doxycycline at 2 μg/mL. ROCK Inhibitor was taken off on the second day and medium was changed daily. Pre-differentiated neurons can be harvested on Day 4 with accutase. Excessive pre-differentiated neurons can be stored in Bambanker (Nippon Genetics, BB01) at 500 k/1 ml in cryovial. Short term storage can be in a −80° C. freezer. Pre-differentiated neurons can be moved to liquid nitrogen after storage overnight at −80° C. freezer for long term storage. For maturation, pre-differentiated neurons were seeded at 90 k/well onto poly-D-lysine (PDL)/laminin coated glass coverslips in 24 well cell culture plate with maturation medium containing 50% DMEM/F12, 50% Neurobasal-A medium (Gibco, 21103049), B27 supplement (Gibco, 17504044), N2 supplement (Gibco, 17502048), GlutaMax (Gibco, 35050061), NEAA (Gibco, 11140050), mouse laminin (Cultrex, 3400-010-01, 1 μg/mL), BDNF (Peprotech 450-02-50UG, 10 ng/mL), and NT3 (Peprotech, 450-03-50UG, 10 ng/mL). Half of the medium was replaced on day 7 and again on day 14, and the medium volume was doubled on day 21. Thereafter, one-third of the medium was replaced weekly until the cells were used.

### Induction of Cortical Astrocytes

The induction process was adapted from previous publication^{59,93}. Briefly, there are three stages of the induction: first stage regional specification of neuroepithelial first differentiated into neuroepithelial cells with or without exogenous patterning molecules (days 0-21). During this stage (Day 1), 70-80% confluent iPSCs were dissociated with accutase for 7 mins and around 1.5 million cells were seeded in 5 ml Essential 8 medium supplemented with ROCK Inhibitor Y-27632 (R&D System, 125410) at 10 μM concentration for forming aggregation of embryo bodies (EB) in a non-coated T25 flask (C6356-200EA). At Day 2, media was switched to astrocyte condition media (DMEM/F12 485 ml, B27 5 ml, N2 2.5 ml and 2 μg/ml heparin (07980, Stem Cell Technologies) with 5 ml Antibiotic-Antimycotic 100× (15240062, Life Technologies) supplemented with SB431542 (2 μM, Tocris, Ellisville, MO) and DMH-1 (200 nM, 412610, R&D System). On Day 2-Day 7, media were changed every other day. EBs could be changed from T25 to T75 due to increasing volume. On Day 8, EB were transferred to Matrigel coated 6 cell culture plates evenly with neural medium (DMEM/F12, NEAA, N2 supplement and heparin with Antibiotic-Antimycotic). Typically, each T75 flask could generate 4 6-well plate worth of EBs. Media were changed every other day with astrocyte condition media. On Day 11 or Day 12, EBs settled down and flatted, forming rosette structures under bright field microscope. On Day 12 or Day 13, through a process named rosette picking, glial progenitor cell clusters were selected. These rosettes were transferred to T25 flasks with wide orifice micropipette tips. Each T25 flask was added with 5 ml astrocyte condition media. From Day 14 to Day 20, media were changed every other day to make sure media did not get too acidic. During this week, flattened picked rosettes would aggregate into spheres and further develop. Large dead cell clusters were disposed in order to keep other clusters healthy. The second stage is the regular dissociation of the neuroepithelial clusters in suspension, permitting the generation of astroglial subtypes over a long-term expansion. From Days 21-Day 90, the astroglial progenitors were either replicated for an extended time or differentiated into functional astrocyte progenitor cells. This process was a propagation and purification process to gain astrocyte progenitor cell population. Astrocyte condition media were added EGF (10 ng/ml, AF-100-15, PeproTech) and FGFP (10 ng/ml, 10778-902, PeproTech). Media were changed every 3-5 days to keep the media pH neutral. When astrosphere attached to the bottom of the flask, either gently resuspended them or if there were enough spheres, they can be disposed and changed for a new flask. During observation of the astrosphere development, if there was dark core inside the sphere, or attached spheres, use accutase to dissociate the spheres and resuspend with astrocyte condition media with EGF and FGFP with ROCK Inhibitor. Around Day 80, there would be a time point when spheres were propagated exponentially, either split into multiple T75 flasks or they could be frozen down with Bambanker (Nippon Genetics, B101). Over 90 days, astrospheres are considered pure astrocyte progenitor cells. From this day on, maintain astrocyte progenitor cells and further mature them. Every week triturate astrospheres by collecting and transferring spheres to a 15 ml canonical tube, allow spheres to settle or centrifuge at 100 g for 90 seconds, then aspirate supernatant and add 1 ml of accutase to spheroid-pellet, gently resuspend, incubate at 37° C. for ˜10 min, use p1000 micropipette to break down the sphere with 5-10 stable agitations. Then allow cells to settle or centrifuge at 100 g for 90 seconds, aspirate supernatant and add 1 ml astrocyte condition media supplemented with ROCK Inhibitor to canonical tube. Ideally, cell suspension should consist of mostly single cells and some small aggregates (3-10 cells each). Repeatedly dissociate astrosphere to further purify astrocyte progenitor cell population until 9 month later. Typically, astrospheres over 9 months old were used for co-culture or tri-culture experiments.

### Induced Neuron 2D Culture

24 well sized autoclaved, PDL (120 μl at 1 mg/ml overnight) and laminin (1 ml at 1 mg/ml) coated glass coverslips were prepared ahead of time. Pre-differentiated neurons were thawed and resuspended with DMEM in a 15 ml canonical tube followed by centrifuged at 300 g for 1.5 mins. After counting, 90 k/well were plated on coverslips with Classic Neuronal Medium containing the following: half DMEM/F12 (Gibco/Thermo Fisher Scientific; Cat. No. 11320-033) and half Neurobasal-A (Gibco/Thermo Fisher Scientific; Cat. No. 10888-022) as the base, 1×MEM Non-Essential Amino Acids, 0.5× GlutaMAX Supplement (Gibco/Thermo Fisher Scientific; Cat. No. 35050-061), 0.5×N2 Supplement, 0.5×B27 Supplement (Gibco/Thermo Fisher Scientific; Cat. No. 17504-044), 10 ng/mL NT-3, 10 ng/mL BDNF, 1 μg/mL Mouse Laminin, and 2 μg/mL doxycycline hydrochloride. Media were changed every other day for 2 weeks.

### Induced Astrocyte 2D Culture

24 well sized autoclaved, PDL (120 μl at 1 mg/ml overnight) and laminin (1 ml at 1 mg/ml) coated glass coverslips were prepared ahead of time. Pre-differentiated astrocytes were dissociated from astrospheres with accutase for 7 mins incubation and resuspended with DMEM in a 15 ml canonical tube followed by centrifuged at 300 g for 1.5 mins. After counting, 20 k/well were plated on coverslips with Classic Neuronal Medium containing the following: astrocyte condition media (DMEM/F12 485 ml, B27 5 ml, N2 2.5 ml and 2 μg/ml heparin (07980, Stem Cell Technologies) with 5 ml Antibiotic-Antimycotic 100× (15240062, Life Technologies) with the addition of ciliary neurotrophic factor (CNTF, 450-13, Peprotech at 2 ng/ml and BMP4, AF-120-05ET, Peprotech, at 2 ng/ml). Media were changed every other day for 2 weeks.

### Induced Neuron and Astrocyte 2D Co-Culture

24 well sized autoclaved, PDL (120 ul at 1 mg/ml overnight) and laminin (1 ml at 1 mg/ml) coated glass coverslips were prepared ahead of time. Pre-differentiated neurons were either dissociated or thawed and resuspended with DMEM in a 15 ml canonical tube followed by centrifuged at 300 g for 1.5 mins. After counting, neurons were plated at the density of 20K/well and were plated. After at least 1-hour, pre-differentiated astrocytes were dissociated from astrospheres with accutase for 7 mins incubation and resuspended with DMEM in a 15 ml canonical tube followed by centrifuged at 300 g for 1.5 mins. After counting, 20 k/well were plated on coverslips with Classic Neuronal Medium containing the following: astrocyte condition media (DMEM/F12 485 ml, B27 5 ml, N2 2.5 ml and 2 μg/ml heparin (07980, Stem Cell Technologies) with 5 ml Antibiotic-Antimycotic 100× (15240062, Life Technologies). Media were changed every other day for 3 weeks.

### Immunocytochemistry of 2D Culture

To examine TDP43 and Ataxin2 localization in induced neuron and induced astrocytes co-culture, as well as neuron, astrocyte 2D mono-culture, immunofluorescent labeling was used. On day 22 for 2D co-culture, 14 for induced neurons, day 7 for induced astrocytes, culture medium was aspirated from each well and cells were subsequently washed with PBS. Cells were fixed with 4% paraformaldehyde, which was prepared by diluting 16% paraformaldehyde (Electron Microscopy Sciences; Cat. No. 15710) 1:4 in PBS, at room temperature for 15 minutes. Paraformaldehyde was removed with a P1000 pipette and collected for proper disposal, and glass coverslips were washed three times with PBS. Cells were blocked with 3% donkey serum in blocking buffer (10% normal goat serum, 3% BSA, 1% glycine and 0.4% Triton X-100) at room temperature for one hour and subsequently incubated with primary antibodies at 4° C. overnight. Coverslips were then washed three times with PBS and incubated with secondary antibodies in blocking buffer at room temperature for one and a half hour. Coverslips were again washed three times with PBS, and incubated with 5 μM DAPI (Thermo Fisher Scientific #D9542-5MG) in TBS at room temperature for 10 minutes. Glass coverslips were then mounted with prolong-gold (Invitrogen, P36934) and air dried.

To examine PRGN localization and knock down condition, on day 7 of 24 well of induced astrocytes, on day 14 of induced neurons, culture media was aspirated and each well and glass coverslips were rinsed with PBS for 3 times. After fixation with 4% paraformaldehyde (Electron Microscopy Sciences Cat. 80827) for 30 mins, glass coverslips with induced neurons were washed with PBS 3 times. Cells were then blocked with 3% donkey serum with 0.1% saponin in PSB (blocking buffer) at room temperature for 1 hour. Then glass coverslips were incubated with goat anti-human PRGN antibody and chicken anti-human p Tubulin Ill antibody in blocking buffer at 4 degree overnight. Cover slips were then washed 3 times in PBS before adding Alexa 488 donkey anti-chicken and Alexa 568 donkey anti-goat secondary antibody in blocking buffer at room temperature for 1.5 hours. After secondary antibody incubation, glass coverslips were washed 3 times in PBS prior to DAPI staining at room temperature for 15 mins. Coverslips were dried by using Kimwipe at the edge and added pro-long Gold Anti-fade (Invitrogen, P36934). We validated all secondary antibodies by negative control experiments omitting the primary antibody, by immunostaining.

To examine PRGN and lysosome co-localization of GRN +/+, GRN −/−, Scramble and GRN Knock Down induced neurons, on day 14 of induced neurons, culture media was aspirated and each well and glass coverslips were rinsed with PBS for 3 times. After fixation with 4% paraformaldehyde (Electron Microscopy Sciences Cat. 80827) for 30 mins, glass coverslips with induced neurons were washed with PBS 3 times. Cells were then blocked with 3% donkey serum with 0.1% saponin in PSB (blocking buffer) at room temperature for 1 hour. Then glass coverslips were incubated with goat anti-human LAMP1 antibody and chicken anti-human p Tubulin Ill antibody in blocking buffer at 4 degree overnight. Cover slips were then washed 3 times in PBS before adding Alexa 488 donkey anti-chicken and Alexa 568 donkey anti-goat secondary antibody in blocking buffer at room temperature for 1.5 hours. After secondary antibody incubation, glass coverslips were washed 3 times in PBS prior to DAPI staining at room temperature for 15 mins. Coverslips were dried by using Kimwipe at the edge and added pro-long Gold Anti-fade (Invitrogen, P36934). We validated all secondary antibodies by negative control experiments omitting the primary antibody, by immunostaining.

### 3D Co-Culture of Neuron and Astrocytes

Co-culture method was adapted from previous publication²⁸. Briefly, 96 well round bottom low attachment culture plates (12456721, Fisher Scientific) were pre-treated with anti-adherent rinsing solution 07010, Stem Cell Technologies) at least 2 hours before seeding the cells. D3 pre-differentiated neurons (WT, GRN knock out, scramble or GRN knock down) and astrocytes (WT or GRN knock out) were seeded roughly 1:2 ratio (540,000 cells in total each well) after optimization with astrocyte condition media (only the initial seeding media include ROCK Inhibitor). After seeding, plates were centrifuged at 300 g to pellet the cells in order to aggregate. For minimal cell death, half of the media was changed every 4-5 days until harvest Day 30.

### Cryosection and Immunohistochemistry of 3D Co-Culture

Assembloids were transferred to a 1.5-ml microcentrifuge tube, using a cut P1000 tip. Media were gently removed. Assembloids were briefly washed with 1 ml of DPBS. DPBS was removed and 500 μl cold 4% (vol/vol) PFA was added to fix for at least 30 min at 4° C. After fixation, PFA was removed and assembloids went through three times 15-min washes with DPBS at RT. After washing, 1 ml of 30% (wt/vol) sucrose was added to facilitate cryoprotection and kept at 4° C. until they equilibrated and sunk to the bottom of the tubes (˜24-48 h). Assembloids were then transferred to embedding solution with 1:1, OCT/30% (wt/vol) sucrose for another 48 h before transferring to 100% OTC and snap-freeze the spheres by placing the mold directly on dry ice. At this point, assembloids blocks were either stored in −80° C. for long term storage or immediate sections. Cryosections were around 40 μm in thickness on glycine coated slides for immunostaining.

Cryosections were briefly washed with TBS and subsequently permeabilized and blocked for fluorescence immunohistochemistry with blocking buffer (10% normal goat serum, 3% BSA, 1% glycine and 0.4% Triton X-100) with 3% normal donkey serum for 1 hr. Primary antibodies were incubated at 4° C. overnight in blocking buffer and appropriate fluorophore conjugated secondary antibodies were applied on the following day for 1.5 hr at RT after three washes with TBS. Sections were then added with DAPI staining at room temperature for 15 mins. Sections were dried by using Kimwipe at edge and added pro-long Gold Anti-fade (Invitrogen, P36934). We validated all secondary antibodies by negative control experiments omitting the primary antibody, by immunostaining.

TABLE 1

| | | Concen- |
|---|---|---|
| Antibody | Vendor and catalog No. | tration |
| TDP43 | Protein Tech, 10782-2-AP | 1:800 |
| BtubulinIII | Millipore Sigma, ab9354 | 1:5000 |
| Ataxin2 | BD Bioscience, 611378 | 1:600 |
| PGRN | R&D System, AF2420 | 1:3000 |
| GFAP | Abcam, ab4674 | 1:1000 |
| LAMP1 | dSHB, H4A3 | 1:1000 |
| PGRN (WB) | ThermoFisher, Cat# | 1:250 |
| | 40-3400 | |
| GAPDH | Sigma-Aldrich, Cat no. | 1:6000 |
| | G8795 | |
| Goat anti-Rabbit IgG (H + L) | Thermo Fisher, Cat# | 1:1000 |
| Poly-HRP Secondary Antibody, | 32260 | |
| HRP | | |
| Goat anti-Mouse IgG (H + L) | Thermo Fisher, Cat# | 1:1000 |
| Poly-HRP Secondary Antibody, | 32230 | |
| HRP | | |
| Goat-Anti-Mouse Alexa 568 | Thermofisher Scientific, | 1:1000 |
| | A11004 | |
| Goat-Anti-Chicken Alexa 647 | Thermofisher Scientific, | 1:1000 |
| | A21449 | |
| Goat-Anti-Rabbit Alexa 488 | Thermofisher Scientific, | 1:1000 |
| | A11008 | |
| Donkey-Anti-Mouse Alexa 647 | Thermofisher Scientific, | 1:1000 |
| | A21202 | |
| Donkey-Anti-Goat Alexa 594 | Thermofisher Scientific, | 1:1000 |
| | A11058 | |
| Donkey-Anti-Chicken CF 633 | Biotium, 20168 | 1:1000 |

### Confocal Image Acquisition and Analysis

3D Assembloid TDP43 Intra/Extra nuclear volume occupancy ratio quantification: Images were collected either on 63× oil immersion objective on Nikon C2 microscope or a Leica SP8 with the oil immersion 63× (NA 1.4) objective. High resolution image stacks of TDP43 labeling were analyzed using a combination of ImageJ and MATLAE R2019b (Mathworks, Inc. Natick, MA) software, the latter implementing custom plotting routines (available at: https://github.com/lucadellasantina/OrganoidImageAnalysis) and VolumeCut, a MATLAEB® app designed for volumetric segmentation and 3D quantification of confocal images (available at: https://lucadellasantina.github.io/VolumeCut/). Briefly, with ImageJ the acquired DAPI signal and TDP43 signals were converted into binary masks using ImageJ's automatic thresholding in order define the nuclear and assembloid volumetric regions, respectively. Using VolumeCut, the raw TDP43 signal intensity and volume occupancy were measured within the assembloids, quantifying separately the intranuclear versus extranuclear compartments. This image analysis pipeline was automated for all the acquired assembloids using the batch processing functionality of VolumeCut. TDP43 volume occupancy was measured for 10 WT co-culture assembloids and 17 GRN KO co-culture assembloids, obtained from 4 repeats of experiments.

3D Assembloid Ataxin2 fluorescence Quantification: 3D images were collected from Leica SP8 and analyzed with Imaris ×64 9.5.1. Briefly, Ataxin2 channel was processed with Gaussian filter and background subtraction. Background fluorescence was determined for each experiments blind to genotypes. Surfaces module was used to obtain Ataxin2 particle number/volume, and Spots module was used to count the number of nuclei in each volume of image.

2D induced neuron TDP43 Intra/Extra nuclear signal ratio analysis quantification: Individual neuron images were collected from Leica SP8 and analyzed within Image J. Intra nuclear, Extra nuclear and background signal in TDP43 channel were measured. Corrected total cell fluorescence (CTCF)=Integrated Density−(Area of selected cell×Mean fluorescence of background readings). Intra nuclear and Extra nuclear CTCF were calculated. The intra/extra nuclear ratio=Intra nuclear TDP43 CTCF/Extra nuclear TDP43 CTCF.

2D induced neuron Ataxin2 fluorescence quantification: Individual neuron images were collected from Leica SP8 and analyzed within Image J. Cytoplasmic and background signal in Ataxin2 channel were measured. Corrected total cell fluorescence (CTCF)=Integrated Density−(Area of selected cell×Mean fluorescence of background readings).

2D Neurite LAMP1 quantification: Individual neuron images were collected from Leica SP8 and analyzed with Imaris ×64 9.5.1. Briefly, LAMP1 channel was analyzed using Spots module, algorithm settings are shortest distance calculation and different spot sizes, with estimated XY diameter threshold 0.3 μm in spot detection. Then signals were excluded within the vicinity of 5-7 μm away from DAPI edges. The remaining count was considered as neurite LAMP1 puncta.

### Astrocyte Phagocytosis Assay

Method has been adapted from previous publication⁹⁴. Briefly, hiPSC-derived astrocytes were plated at approximately 200 k cells per well in a 24-well Matrigel coated plate and allowed to grow for 1 weeks prior to experiments in Astrosphere media supplemented with CNTF and BMP4. Zymosan conjugated with a pHrodo red dye (Thermo Fisher, P35364) is dissolved in PBS at 1 mg/ml and sonicated for 10 minutes to break down large chunks. Astrocytes are incubated with 30 μg of pHrodo-labeled Zymosan (50 μl reconstituted) for 3 hours. Cells are then dissociated with accutase from the culture plate, washed with PBS once and resuspended in 500 μl of 1% BSA in PBS, then DAPI is added at 1:1000 dilution for 5 mins. There are three controls, one control is astrocytes in PBS only without DAPI or Zymosan, one is just added DAPI (1:1000 dilution) for 5 mins, one is astrocytes only incubated with Zymosan for 3 hours. Cells were kept on ice and immediately analyzed by flow cytometry. Experiments are repeated at least 5 times. All flow cytometry data were acquired on a LSRII (BD Biosciences) and analyzed using FlowJo software. Median Fluorescent Intensity (MFI) was plotted between GRN +/+ and GRN −/− induced astrocytes.

### RNA Isolation and RT-qPCR

In order to confirm GRN KO and assess GRN knock down level, RT PCR was performed on WT, GRN KO, scramble and GRN knock down iPSCs. RNAs from iPSC of different genotypes were extracted using BioRad Aurum Total RNA Mini Kit (7326820) following manufacturer's instructions with three replicates of iPSCs. Samples were prepared for iTaq™ Universal SYBR® Green One-Step Kit (1725150), as well as the PCR cycles using a Bio-Rad C1000 Thermal Cycler/CF96 Real-Time System.

GRN_F
(SEQ ID NO: 3)
TCCAGAGTAAGTGCCTCTCCA
GRN R
(SEQ ID NO: 4)
TCACCTCCATGTCACATTTCA
GAPDH_F
(SEQ ID NO: 5)
ATGGGGAAGGTGAAGGTCG
GAPDH_R
(SEQ ID NO: 6)
GGGGTCATTGATGGCAACAATA

### Quantification of Knock Down by Western Blotting

To quantify protein level knockdown of GRN by GRN sgRNA in CRISPRi-iPSCs, iPSCs with 1 non-targeting sgRNAs (Scramble), along with WT and GRN KO iPSCs were lysed and 20 ug of total protein from each lysate was loaded into a NuPAGE 4%-12% Bis-Tris Gel (Invitrogen, Cat #NP0336BOX). Subsequently, the gel was transferred onto a nitrocellulose membrane, which was then blocked in 5% milk in PBS-T (PBS with 0.02% tween), followed by overnight incubation with primary antibodies at 4 degree. The primary antibodies used were Mouse monoclonal anti-GAPDH (Sigma-Aldrich, Cat no. G8795) and Rabbit monoclonal anti-GRN (ThermoFisher, cat #40-3400). After incubation, the membrane was washed three times with PBS-T and then incubated with secondary antibodies at room temperature for 1 hr. The membrane was then washed 3 times with PBS-T, incubated for two minutes with Pierce ECL western blotting substrate (Thermo Fisher Cat #32106) and imaged at ChemiDoc (BioRad). Digital images were processed and analyzed using the image analysis software, ImageJ.

### Statistical Analysis

For all experiments, data are represented as mean±SEM of at least three independent experiments. We compared groups using a Mann-Whitney U test for all analysis except FIG. 3D where a paired-t-test was used. Significant comparisons are labeled with * p<0.05, ** p<0.01, *** p<0.001, **** p<0.0001. GraphPad Prism version 8.4.2 was used for statistical analyses.

### REFERENCES

1 Dawson, T. M., Golde, T. E. & Lagier-Tourenne, C. Animal models of neurodegenerative diseases. Nat Neurosci 21, 1370-1379, doi:10.1038/s41593-018-0236-8 (2018).
2 Deep-brain stimulation of the subthalamic nucleus or the pars interna of the globus pallidus in Parkinson's disease. N Eng J Med 345, 956-963, doi:10.1056/NEJMoa000827 (2001).
3 Kao, A. W., McKay, A., Singh, P. P., Brunet, A. & Huang, E. J. Progranulin, lysosomal regulation and neurodegenerative disease. Nat Rev Neurosci 18, 325-333, doi:10.1038/nrn.2017.36 (2017).
4 Choi, S. H. et al. A three-dimensional human neural cell culture model of Alzheimer's disease. Nature 515, 274-278, doi:10.1038/nature13800 (2014).
Gonzalez, C. et al. Modeling amyloid beta and tau pathology in human cerebral organoids. Mol Psychiatry 23, 2363-2374, doi:10.1038/s41380-018-0229-8 (2018).
6 Kim, Y. H. et al. A 3D human neural cell culture system for modeling Alzheimer's disease. Nat Protoc 10, 985-1006, doi:10.1038/nprot.2015.065 (2015).
7 Kwak, S. S. et al. Amyloid-beta42/40 ratio drives tau pathology in 3D human neural cell culture models of Alzheimer's disease. Nat Commun 11, 1377, doi:10.1038/s41467-020-15120-3 (2020).
8 Lee, H. K. et al. Three Dimensional Human Neuro-Spheroid Model of Alzheimer's Disease Based on Differentiated Induced Pluripotent Stem Cells. PLoS One 11, e0163072, doi:10.1371/journal.pone.0163072 (2016).
9 Yan, Y. et al. Modeling Neurodegenerative Microenvironment Using Cortical Organoids Derived from Human Stem Cells. Tissue Eng Part A 24, 1125-1137, doi:10.1089/ten.TEA.2017.0423 (2018).
10 Penney, J., Ralvenius, W. T. & Tsai, L. H. Modeling Alzheimer's disease with iPSC-derived brain cells. Mol Psychiatry 25, 148-167, doi:10.1038/s41380-019-0468-3 (2020).
11 Hernandez-Sapiens, M. A. et al. A Three-Dimensional Alzheimer's Disease Cell Culture Model Using iPSC-Derived Neurons Carrying A246E Mutation in PSEN1. Front Cell Neurosci 14, 151, doi:10.3389/fncel.2020.00151 (2020).
12 Slanzi, A., Iannoto, G., Rossi, B., Zenaro, E. & Constantin, G. In vitro Models of Neurodegenerative Diseases. Front Cell Dev Biol 8, 328, doi:10.3389/fcell.2020.00328 (2020).
13 Choi, S. H., Kim, Y. H., Quinti, L., Tanzi, R. E. & Kim, D. Y. 3D culture models of Alzheimer's disease: a road map to a “cure-in-a-dish”. Mol Neurodegener 11, 75, doi:10.1186/s13024-016-0139-7 (2016).
14 Wu, Y. Y., Chiu, F. L., Yeh, C. S. & Kuo, H. C. Opportunities and challenges for the use of induced pluripotent stem cells in modelling neurodegenerative disease. Open Biol 9, 180177, doi:10.1098/rsob.180177 (2019).
15 Mofazzal Jahromi, M. A. et al. Microfluidic Brain-on-a-Chip: Perspectives for Mimicking Neural System Disorders. Mol Neurobiol 56, 8489-8512, doi:10.1007/s12035-019-01653-2 (2019).
16 Bordoni, M. et al. From Neuronal Differentiation of iPSCs to 3D Neuro-Organoids: Modelling and Therapy of Neurodegenerative Diseases. Int J Mol Sci 19, doi:10.3390/ijms19123972 (2018).
17 Korhonen, P., Malm, T. & White, A. R. 3D human brain cell models: New frontiers in disease understanding and drug discovery for neurodegenerative diseases. Neurochem Int 120, 191-199, doi:10.1016/j.neuint.2018.08.012 (2018).
18 Centeno, E. G. Z., Cimarosti, H. & Bithell, A. 2D versus 3D human induced pluripotent stem cell-derived cultures for neurodegenerative disease modelling. Mol Neurodegener 13, 27, doi:10.1186/s13024-018-0258-4 (2018).
19 Poon, A. et al. Modeling neurodegenerative diseases with patient-derived induced pluripotent cells: Possibilities and challenges. N Biotechnol 39, 190-198, doi:10.1016/j.nbt.2017.05.009 (2017).
20 Lee, C. T., Bendriem, R. M., Wu, W. W. & Shen, R. F. 3D brain Organoids derived from pluripotent stem cells: promising experimental models for brain development and neurodegenerative disorders. J Biomed Sci 24, 59, doi:10.1186/s12929-017-0362-8 (2017).
21 Son, M. Y. et al. Distinctive genomic signature of neural and intestinal organoids from familial Parkinson's disease patient-derived induced pluripotent stem cells. Neuropathol Appl Neurobiol 43, 584-603, doi:10.1111/nan.12396 (2017).
22 Kim, H. et al. Modeling G2019S-LRRK2 Sporadic Parkinson's Disease in 3D Midbrain Organoids. Stem Cell Reports 12, 518-531, doi:10.1016/j.stemcr.2019.01.020 (2019).
23 Miguel, L. et al. Detection of all adult Tau isoforms in a 3D culture model of iPSC-derived neurons. Stem Cell Res 40, 101541, doi:10.1016/j.scr.2019.101541 (2019).
24 Bolognin, S. et al. 3D Cultures of Parkinson's Disease-Specific Dopaminergic Neurons for High Content Phenotyping and Drug Testing. Adv Sci (Weinh) 6, 1800927, doi:10.1002/advs.201800927 (2019).
25 Arai, T. et al. TDP-43 is a component of ubiquitin-positive tau-negative inclusions in frontotemporal lobar degeneration and amyotrophic lateral sclerosis. Biochem Biophys Res Commun 351, 602-611, doi:10.1016/j.bbrc.2006.10.093 (2006).
26 Neumann, M. et al. Ubiquitinated TDP-43 in frontotemporal lobar degeneration and amyotrophic lateral sclerosis. Science 314, 130-133, doi:10.1126/science.1134108 (2006).
27 Vatsavayai, S. C., Nana, A. L., Yokoyama, J. S. & Seeley, W. W. C9orf72-FTD/ALS pathogenesis: evidence from human neuropathological studies. Acta Neuropathol 137, 1-26, doi:10.1007/s00401-018-1921-0 (2019).
28 Krencik, R. et al. Systematic Three-Dimensional Coculture Rapidly Recapitulates Interactions between Human Neurons and Astrocytes. Stem Cell Reports 9, 1745-1753, doi:10.1016/j.stemcr.2017.10.026 (2017).
29 Cruts, M. et al. Null mutations in progranulin cause ubiquitin-positive frontotemporal dementia linked to chromosome 17q21. Nature 442, 920-924, doi:10.1038/nature05017 (2006).
30 Lui, H. et al. Progranulin Deficiency Promotes Circuit-Specific Synaptic Pruning by Microglia via Complement Activation. Cell 165, 921-935, doi:10.1016/j.cell.2016.04.001 (2016).
31 Ward, M. E. et al. Early retinal neurodegeneration and impaired Ran-mediated nuclear import of TDP-43 in progranulin-deficient FTLD. J Exp Med 211, 1937-1945, doi:10.1084/jem.20140214 (2014).
32 Nguyen, A. D. et al. Murine knockin model for progranulin-deficient frontotemporal dementia with nonsense-mediated mRNA decay. Proc Natl Acad Sci USA 115, E2849-E2858, doi:10.1073/pnas.1722344115 (2018).
33 Zhang, J. et al. Neurotoxic microglia promote TDP-43 proteinopathy in progranulin deficiency. Nature 588, 459-465, doi:10.1038/s41586-020-2709-7 (2020).
34 Ward, M. E. et al. Individuals with progranulin haploinsufficiency exhibit features of neuronal ceroid lipofuscinosis. Sci Transl Med 9, doi:10.1126/scitranslmed.aah5642 (2017).
35 Rosen, E. Y. et al. Functional genomic analyses identify pathways dysregulated by progranulin deficiency, implicating Wnt signaling. Neuron 71, 1030-1042, doi:10.1016/j.neuron.2011.07.021 (2011).
36 Lee, S. & Huang, E. J. Modeling ALS and FTD with iPSC-derived neurons. Brain Res 1656, 88-97, doi:10.1016/j.brainres.2015.10.003 (2017).
37 Almeida, S. et al. Induced pluripotent stem cell models of progranulin-deficient frontotemporal dementia uncover specific reversible neuronal defects. Cell Rep 2, 789-798, doi:10.1016/j.celrep.2012.09.007 (2012).
38 Ahmed, Z. et al. Accelerated lipofuscinosis and ubiquitination in granulin knockout mice suggest a role for progranulin in successful aging. Am J Pathol 177, 311-324, doi:10.2353/ajpath.2010.090915 (2010).
39 Kelley, K. W., Nakao-Inoue, H., Molofsky, A. V. & Oldham, M. C. Variation among intact tissue samples reveals the core transcriptional features of human CNS cell classes. Nat Neurosci 21, 1171-1184, doi:10.1038/s41593-018-0216-z (2018).
40 Zhang, Y. et al. Purification and Characterization of Progenitor and Mature Human Astrocytes Reveals Transcriptional and Functional Differences with Mouse. Neuron 89, 37-53, doi:10.1016/j.neuron.2015.11.013 (2016).
41 Zhang, Y. et al. An RNA-sequencing transcriptome and splicing database of glia, neurons, and vascular cells of the cerebral cortex. J Neurosci 34, 11929-11947, doi:10.1523/JNEUROSCI.1860-14.2014 (2014).
42 Hansen, D. V., Hanson, J. E. & Sheng, M. Microglia in Alzheimer's disease. J Cell Biol 217, 459-472, doi:10.1083/jcb.201709069 (2018).
43 Verkhratsky, A., Olabarria, M., Noristani, H. N., Yeh, C. Y. & Rodriguez, J. J. Astrocytes in Alzheimer's disease. Neurotherapeutics 7, 399-412, doi:10.1016/j.nurt.2010.05.017 (2010).
44 Desai, M. K. et al. Early oligodendrocyte/myelin pathology in Alzheimer's disease mice constitutes a novel therapeutic target. Am J Pathol 177, 1422-1435, doi:10.2353/ajpath.2010.100087 (2010).
45 Tian, R. et al. CRISPR Interference-Based Platform for Multimodal Genetic Screens in Human iPSC-Derived Neurons. Neuron 104, 239-255 e212, doi:10.1016/j.neuron.2019.07.014 (2019).
46 Wang, C. et al. Scalable Production of iPSC-Derived Human Neurons to Identify Tau-Lowering Compounds by High-Content Screening. Stem Cell Reports 9, 1221-1233, doi:10.1016/j.stemcr.2017.08.019 (2017).
47 Fernandopulle, M. S. et al. Transcription Factor-Mediated Differentiation of Human iPSCs into Neurons. Curr Protoc Cell Biol 79, e51, doi:10.1002/cpcb.51 (2018).
48 Zhang, Y. et al. Rapid single-step induction of functional neurons from human pluripotent stem cells. Neuron 78, 785-798, doi:10.1016/j.neuron.2013.05.029 (2013).
49 Davidson, Y. et al. Ubiquitinated pathological lesions in frontotemporal lobar degeneration contain the TAR DNA-binding protein, TDP-43. Acta Neuropathol 113, 521-533, doi:10.1007/s00401-006-0189-y (2007).
50 de Majo, M., Koontz, M., Rowitch, D. & Ullian, E. M. An update on human astrocytes and their role in development and disease. Glia 68, 685-704, doi:10.1002/glia.23771 (2020).
51 Nonhoff, U. et al. Ataxin-2 interacts with the DEAD/H-box RNA helicase DDX6 and interferes with P-bodies and stress granules. Mol Biol Cell 18, 1385-1396, doi:10.1091/mbc.e06-12-1120 (2007).
52 Kaehler, C. et al. Ataxin-2-like is a regulator of stress granules and processing bodies. PLoS One 7, e50134, doi:10.1371/journal.pone.0050134 (2012).
53 Li, Y. R., King, O. D., Shorter, J. & Gitler, A. D. Stress granules as crucibles of ALS pathogenesis. J Cell Biol 201, 361-372, doi:10.1083/jcb.201302044 (2013).
54 Khalfallah, Y. et al. TDP-43 regulation of stress granule dynamics in neurodegenerative disease-relevant cell types. Sci Rep 8, 7551, doi:10.1038/s41598-018-25767-0 (2018).
55 Becker, L. A. et al. Therapeutic reduction of ataxin-2 extends lifespan and reduces pathology in TDP-43 mice. Nature 544, 367-371, doi:10.1038/nature22038 (2017).
56 Feng, T. et al. Loss of TMEM106B and PGRN leads to severe lysosomal abnormalities and neurodegeneration in mice. EMBO Rep 21, e50219, doi:10.15252/embr.202050219 (2020).
57 Zhou, X. et al. Loss of Tmem106b exacerbates FTLD pathologies and causes motor deficits in progranulin-deficient mice. EMBO Rep 21, e50197, doi:10.15252/embr.202050197 (2020).
58 Tayebi, N., Lopez, G., Do, J. & Sidransky, E. Pro-cathepsin D, Prosaposin, and Progranulin: Lysosomal Networks in Parkinsonism. Trends Mol Med 26, 913-923, doi:10.1016/j.molmed.2020.07.004 (2020).
59 Krencik, R. & Zhang, S. C. Directed differentiation of functional astroglial subtypes from human pluripotent stem cells. Nat Protoc 6, 1710-1717, doi:10.1038/nprot.2011.405 (2011).
60 di Domenico, A. et al. Patient-Specific iPSC-Derived Astrocytes Contribute to Non-Cell-Autonomous Neurodegeneration in Parkinson's Disease. Stem Cell Reports 12, 213-229, doi:10.1016/j.stemcr.2018.12.011 (2019).
61 Aflaki, E. et al. A characterization of Gaucher iPS-derived astrocytes: Potential implications for Parkinson's disease. Neurobiol Dis 134, 104647, doi:10.1016/j.nbd.2019.104647 (2020).
62 Barbar, L. et al. CD49f Is a Novel Marker of Functional and Reactive Human iPSC-Derived Astrocytes. Neuron 107, 436-453 e412, doi:10.1016/j.neuron.2020.05.014 (2020).
63 Zamanian, J. L. et al. Genomic analysis of reactive astrogliosis. J Neurosci 32, 6391-6410, doi:10.1523/JNEUROSCI.6221-11.2012 (2012).
64 Liddelow, S. A. et al. Neurotoxic reactive astrocytes are induced by activated microglia. Nature 541, 481-487, doi:10.1038/nature21029 (2017).
65 Herculano-Houzel, S. The glia/neuron ratio: how it varies uniformly across brain structures and species and what that means for brain physiology and evolution. Glia 62, 1377-1391, doi:10.1002/glia.22683 (2014).
66 Pelvig, D. P., Pakkenberg, H., Stark, A. K. & Pakkenberg, B. Neocortical glial cell numbers in human brains. Neurobiol Aging 29, 1754-1762, doi:10.1016/j.neurobiolaging.2007.04.013 (2008).
67 Mackenzie, I. R. & Rademakers, R. The role of transactive response DNA-binding protein-43 in amyotrophic lateral sclerosis and frontotemporal dementia. Curr Opin Neurol 21, 693-700, doi:10.1097/WCO.0b013e3283168d1d (2008).
68 Auburger, G., Sen, N. E., Meierhofer, D., Basak, A. N. & Gitler, A. D. Efficient Prevention of Neurodegenerative Diseases by Depletion of Starvation Response Factor Ataxin-2. Trends Neurosci 40, 507-516, doi:10.1016/j.tins.2017.06.004 (2017).
69 Zhang, D. et al. A 3D Alzheimer's disease culture model and the induction of P21-activated kinase mediated sensing in iPSC derived neurons. Biomaterials 35, 1420-1428, doi:10.1016/j.biomaterials.2013.11.028 (2014).
70 Kampmann, M. A CRISPR Approach to Neurodegenerative Diseases. Trends Mol Med 23, 483-485, doi:10.1016/j.molmed.2017.04.003 (2017).
71 Horlbeck, M. A. et al. Compact and highly active next-generation libraries for CRISPR-mediated gene repression and activation. Elife 5, doi:10.7554/eLife.19760 (2016).
72 Chen-Plotkin, A. S. et al. Brain progranulin expression in GRN-associated frontotemporal lobar degeneration. Acta Neuropathol 119, 111-122, doi:10.1007/s00401-009-0576-2 (2010).
73 Hosaka, T. et al. A novel frameshift GRN mutation results in frontotemporal lobar degeneration with a distinct clinical phenotype in two siblings: case report and literature review. BMC Neurol 17, 182, doi:10.1186/s12883-017-0959-2 (2017).
74 Wauters, E., Van Mossevelde, S., Van der Zee, J., Cruts, M. & Van Broeckhoven, C. Modifiers of GRN-Associated Frontotemporal Lobar Degeneration. Trends Mol Med 23, 962-979, doi:10.1016/j.molmed.2017.08.004 (2017).
75 Holler, C. J. et al. Trehalose upregulates progranulin expression in human and mouse models of GRN haploinsufficiency: a novel therapeutic lead to treat frontotemporal dementia. Mol Neurodegener 11, 46, doi:10.1186/s13024-016-0114-3 (2016).
76 Hester, M. E. et al. Rapid and efficient generation of functional motor neurons from human pluripotent stem cells using gene delivered transcription factor codes. Mol Ther 19, 1905-1912, doi:10.1038/mt.2011.135 (2011).
77 Shi, Y. et al. Haploinsufficiency leads to neurodegeneration in C90RF72 ALS/FTD human induced motor neurons. Nat Med 24, 313-325, doi:10.1038/nm.4490 (2018).
78 Yang, N. et al. Generation of pure GABAergic neurons by transcription factor programming. Nat Methods 14, 621-628, doi:10.1038/nmeth.4291 (2017).
79 Muffat, J. et al. Efficient derivation of microglia-like cells from human pluripotent stem cells. Nat Med 22, 1358-1367, doi:10.1038/nm.4189 (2016).
80 Pandya, H. et al. Differentiation of human and murine induced pluripotent stem cells to microglia-like cells. Nat Neurosci 20, 753-759, doi:10.1038/nn.4534 (2017).
81 Douvaras, P. et al. Directed Differentiation of Human Pluripotent Stem Cells to Microglia. Stem Cell Reports 8, 1516-1524, doi:10.1016/j.stemcr.2017.04.023 (2017).
82 Abud, E. M. et al. iPSC-Derived Human Microglia-like Cells to Study Neurological Diseases. Neuron 94, 278-293 e279, doi:10.1016/j.neuron.2017.03.042 (2017).
83 Xu, R. et al. Human iPSC-derived mature microglia retain their identity and functionally integrate in the chimeric mouse brain. Nat Commun 11, 1577, doi:10.1038/s41467-020-15411-9 (2020).
84 Haenseler, W. et al. A Highly Efficient Human Pluripotent Stem Cell Microglia Model Displays a Neuronal-Co-culture-Specific Expression Profile and Inflammatory Response. Stem Cell Reports 8, 1727-1742, doi:10.1016/j.stemcr.2017.05.017 (2017).
85 Mantle, J. L. & Lee, K. H. A differentiating neural stem cell-derived astrocytic population mitigates the inflammatory effects of TNF-alpha and IL-6 in an iPSC-based blood-brain barrier model. Neurobiol Dis 119, 113-120, doi:10.1016/j.nbd.2018.07.030 (2018).
86 Canfield, S. G. et al. An isogenic blood-brain barrier model comprising brain endothelial cells, astrocytes, and neurons derived from human induced pluripotent stem cells. J Neurochem 140, 874-888, doi:10.1111/jnc.13923 (2017).
87 Appelt-Menzel, A. et al. Establishment of a Human Blood-Brain Barrier Co-culture Model Mimicking the Neurovascular Unit Using Induced Pluri- and Multipotent Stem Cells. Stem Cell Reports 8, 894-906, doi:10.1016/j.stemcr.2017.02.021 (2017).
88 Qian, T. et al. Directed differentiation of human pluripotent stem cells to blood-brain barrier endothelial cells. Sci Adv 3, e1701679, doi:10.1126/sciadv.1701679 (2017).
89 Lippmann, E. S. et al. Derivation of blood-brain barrier endothelial cells from human pluripotent stem cells. Nat Biotechnol 30, 783-791, doi:10.1038/nbt.2247 (2012).
90 Orlova, V. V. et al. Functionality of endothelial cells and pericytes from human pluripotent stem cells demonstrated in cultured vascular plexus and zebrafish xenografts. Arterioscler Thromb Vasc Biol 34, 177-186, doi:10.1161/ATVBAHA.113.302598 (2014).
91 Kumar, A. et al. Specification and Diversification of Pericytes and Smooth Muscle Cells from Mesenchymoangioblasts. Cell Rep 19, 1902-1916, doi:10.1016/j.celrep.2017.05.019 (2017).
92 Blanchard, J. W. et al. Reconstruction of the human blood-brain barrier in vitro reveals a pathogenic mechanism of APOE4 in pericytes. Nat Med 26, 952-963, doi:10.1038/s41591-020-0886-4 (2020).
93 Krencik, R., Weick, J. P., Liu, Y., Zhang, Z. J. & Zhang, S. C. Specification of transplantable astroglial subtypes from human pluripotent stem cells. Nat Biotechnol 29, 528-534, doi:10.1038/nbt.1877 (2011).
94 Tcw, J. et al. An Efficient Platform for Astrocyte Differentiation from Human Induced Pluripotent Stem Cells. Stem Cell Reports 9, 600-614, doi:10.1016/j.stemcr.2017.06.018 (2017).

### Example 2

### Assembled 3D Cultures of Human Neurons and Microglia

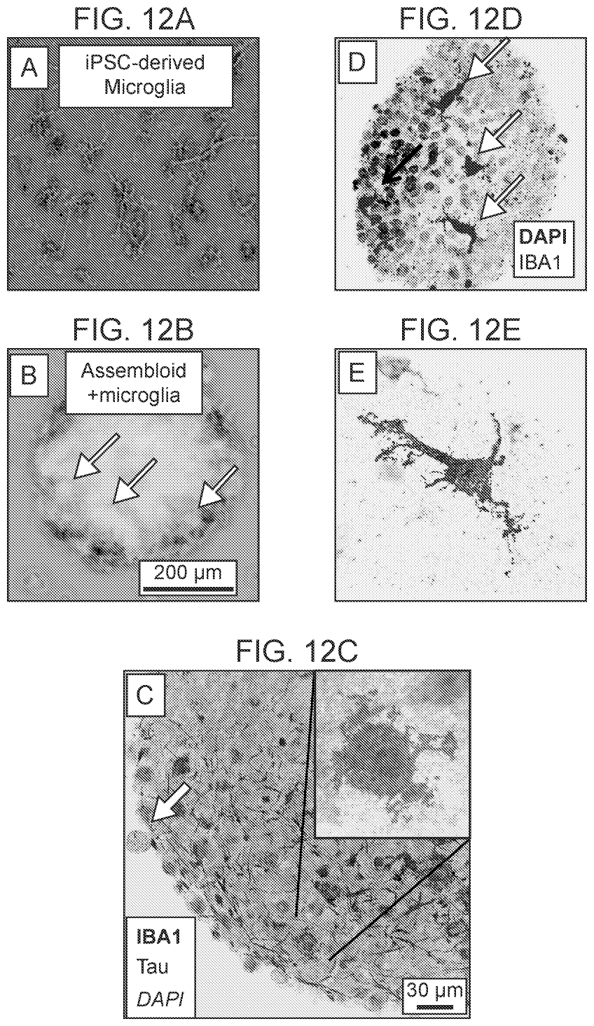

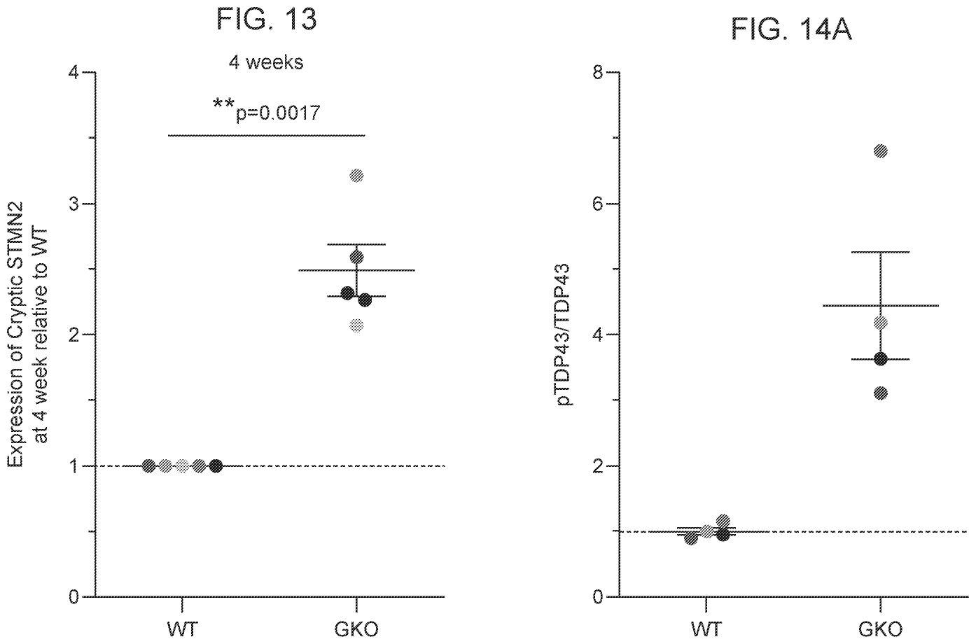

3D mAssembloids were generated containing neurons and microglia (FIGS. 12A-12E). FIG. 12A shows iPSC-derived microglia in a 2D conventional culture. FIG. 12B. shows incorporation of iPSC-derived microglia into a mAssembloid imaged with conventional phase light microscopy. The mAssembloid was immunostained for neuronal Tau (red) and microglial IBA1 (green) 24 hours after addition of the microglia (FIG. 12C). Immunostaining showed that numerous microglia were present in the mAssembloid with extending processes. FIG. 12D shows a 30-micron section of the mAssembloid 30 days after microglia incorporation. Numerous cell bodies and processes were seen in the section. FIG. 12E shows a computer reconstruction of a microglial cell in a 30-micron section. Numerous processes were seen in the section and the microglial cell exhibits a resting-like morphology indicating a non-activated state.

### Example 3

### Midbrain, Parkinson's Disease Model

### Methodology:

A. Generate Dopamine Neurons Using PB6F:

To generate dopaminergic (DA) neurons using the PB6F approach, we use the plasmids from Dr. Marius Wernig's lab and piggyBac electroporation and selection as described by Ng et al. (Stem Cell Reports (2021) 16(7):1763-1776; herein incorporated by reference in its entirety) to generate stable transcription factor expressing cell lines. This method requires the use of 5 plasmids (including the transposase) and yields cell lines that can be expanded in about 7-10 days. These cell lines can be differentiated into about 20% DA neurons using doxycline. The DA neurons are useable in another approximately 21 days.

B. Generate Ventral Midbrain Astrocytes with Chir99021 and Purmorphamine

To generate ventral midbrain astrocytes, we employ our published methods based on the protocol by Krencik et al., (2015). This protocol generates neural rosettes that can be specified to various CNS regions including ventral midbrain using a combination of Chir99021 and Purmorphamine and then differentiated to astrocytes (similar to Krencik et al. (2011) Nat. Protoc. 6(11):1710-1717 (PMC: 3198813); herein incorporated by reference in its entirety).

C. Generate Microglia

Microglia are generated using the method of Abud et al. (Neuron (2017) 94(2):278-293.e9 (PMID: 28426964; herein incorporated by reference in its entirety). Briefly iPSCs are differentiated into the haemopoietic lineage and after about 35 days yield mature microglial-like cells. These cells can be made from a variety of iPSC lines and are differentiated from the lines listed above.

iPSC-derived induced microglia-like cells (iMGL) are generated within less than 6 weeks using a defined protocol by Abud et al., supra). The two-step protocol starts with the differentiation of hiPSC lines to hematopoietic progenitor cells (iHPCs), that recapitulates the early primitive cells derived from the yolk sac that give rise to microglia during embryo development. This robust protocol yields CD43+/CD235a+/CD41+ iHPC cells that can be further differentiated into microglia-like cells, by growing in a defined serum-free media made in-house, supplemented with three cytokines: M-CSF1, TGFβ and IL-34. The last step including an attempt to recapitulate soluble CNS cues present in the brain by including signals derived from other cell types in the brain that interact with microglia to influence gene expression and function. These include CX3CL1, CD200, and TGFβ. Over the course of 38 days, iMGL cells are matured and form ramified morphology similar to in vivo microglia.

D. Generate Assembloids

3D co-culture of Neuron, Astrocytes, and Microglia: Briefly, 96 well round bottom low attachment culture plates (Fisher Scientific Cat. 12456721) are pre-treated with anti-adherent rinsing solution (Stem Cell Technologies Cat. 07010) at least 2 hours before seeding the cells. PB6F pre-differentiated neurons and astrocytes are seeded at a roughly 2:1 ratio (approx. 50,000 cells in total each well) after initial culture period with astrocyte condition media (only the initial seeding media also includes ROCK Inhibitor). After seeding, plates are centrifuged at 300 g to pellet the cells in order to aggregate. Microglia are reseeded at 10,000 cells per assembloid at days 10-14 and allowed to migrate into the assembloid where they proliferate and tile into densities similar to those found in the CNS (200 cells/mm²). A 50% media change is performed every 4-5 days until harvest at Day 30.

E. Assembloid Parkinson's Disease Model

To create a Parkinson's disease model, assembloids comprising neuron, astrocytes, and microglia derived from cell lines comprising one or more mutations linked to Parkinson's disease are generated. Mutations assessed include an SNCA variant A53T, ATP13A2 variant c1306, and the SYNJ1 variant R219Q.

What is claimed is:

1. A method of producing an assembled three-dimensional organoid comprising mature neurons and mature glia, the method comprising:
a) isolating mature induced pluripotent stem cell (IPSC)-derived neurons from a first cell population and isolating mature IPSC-derived glia from a second cell population;
b) combining a selected number of the mature IPSC-derived neurons and the mature IPSC-derived glia to produce a mixed culture having the mature IPSC-derived neurons and the mature IPSC-derived glia at a selected ratio;
c) aggregating the mature IPSC-derived neurons and the mature IPSC-derived glia; and
d) culturing the aggregated IPSC-derived neurons and IPSC-derived glia, wherein the culturing results in generation of the assembled three-dimensional organoid.

2. The method of claim 1, wherein the mature IPSC-derived neurons are interneurons, motor neurons, sensory neurons, afferent neurons, efferent neurons, inhibitory neurons, or excitatory neurons, or any combination thereof.

3. The method of claim 1, wherein the mature IPSC-derived neurons are glutamatergic neurons, cholinergic neurons, GABAergic neurons, dopaminergic neurons, serotonergic neurons, or histaminergic neurons, or any combination thereof.

4. The method of any one of claims 1 to 3, wherein the mature IPSC-derived neurons are produced by a method comprising:
a) pre-differentiating IPSCs in pre-differentiation media comprising master neuronal transcriptional regulator neurogenin-2 (NGN2) and a rho-associated protein kinase (ROCK) inhibitor, wherein pre-differentiated neurons are produced; and
b) culturing the pre-differentiated neurons in maturation media comprising brain-derived neurotrophic factor (BDNF) and neurotrophin-3 (NT3), wherein mature IPSC-derived neurons are produced.

5. The method of any one of claims 1 to 4, wherein the mature IPSC-derived glia are astrocytes, oligodendrocytes, ependymal cells, microglia, NG2 glia, or any combination thereof.

6. The method of claim 5, wherein the mature IPSC-derived astrocytes are produced by a method comprising:
a) differentiating IPSCs into neuroepithelial cells in neural media comprising a ROCK inhibitor, wherein the neuroepithelial cells aggregate into embryo bodies;
b) differentiating neuroepithelial cells in astrocyte condition media comprising epidermal growth factor (EGF) and basic fibroblast growth factor (FGFβ), wherein astrospheres comprising astrocyte progenitor cells are produced; and
c) maturing astrocyte progenitor cells by culturing astrospheres in the astrocyte condition media for at least 9 months, wherein mature IPSC-derived astrocytes are produced.

7. The method of any one of claims 1 to 6, wherein the mature IPSC-derived neurons or the mature IPSC-derived glia or both the mature IPSC-derived neurons and the mature IPSC-derived glia comprise at least one genetic mutation associated with a neurological disorder, a neurodevelopmental disorder, or a neurodegenerative disease.

8. The method of claim 7, wherein said at least one genetic mutation is a GRN mutation associated with frontotemporal dementia or lipofusis.

9. The method of claim 7 or 8, where said at least one genetic mutation results in knockdown or knockout of a GRN gene.

10. The method of any one of claims 1 to 9, further comprising using a CRISPR system to make genetic changes to a gene of interest in the mature IPSC-derived neurons or the mature IPSC-derived glia, or the IPSCs or progenitor cells from which they are derived.

11. The method of claim 10, wherein the CRISPR system is used to knockdown or knockout a GRN gene in the mature IPSC-derived neurons or the mature IPSC-derived glia.

12. The method of claim 11, wherein the CRISPR system comprises a GRN guide RNA (gRNA) comprising the sequence of SEQ ID NO:1, or a gRNA having up to three nucleotide changes in the nucleotide sequence of SEQ ID NO:1, wherein the gRNA is capable of hybridizing to a target GRN gene sequence.

13. The method of any one of claims 1 to 12, wherein the mature IPSC-derived neurons or the mature IPSC-derived glia or both the mature IPSC-derived neurons and the mature IPSC-derived glia are generated from IPSCs comprising at least one genetic mutation associated with a neurological disorder, a neurodevelopmental disorder, or a neurodegenerative disease.

14. The method of any one of claims 1 to 12, further comprising:
a) collecting somatic cells from a patient having at least one genetic mutation associated with a neurological disorder, a neurodevelopmental disorder, or a neurodegenerative disease;
b) generating IPSCs from the somatic cells; and
c) differentiating the IPSCs to produce the first cell population comprising the mature IPSC-derived neurons or the second cell population comprising the mature IPSC-derived glia, or both the first cell population comprising the mature IPSC-derived neurons and the second cell population comprising the mature IPSC-derived glia.

15. The method of any one of claims 1 to 12, further comprising genetically modifying the mature IPSC-derived neurons or the mature IPSC-derived glia or both the mature IPSC-derived neurons and the mature IPSC-derived glia to introduce at least one genetic mutation associated with a neurological disorder, a neurodevelopmental disorder, or a neurodegenerative disease into their genome.

16. The method of any one of claims 1 to 15, wherein the mature IPSC-derived neurons and the mature IPSC-derived glia are generated from IPSCs derived from cells from the same source.

17. The method of any one of claims 1 to 16, wherein the selected ratio of the mature IPSC-derived neurons to the mature IPSC-derived glia is a 2:1, 1:1, 1:2, 1:3, or 1:4 ratio.

18. The method of any one of claims 1 to 17, wherein said culturing is performed in a non-adherent container.

19. The method of any one of claims 1 to 18, wherein said aggregating comprising centrifuging the mixed culture.

20. The method of any one of claims 1 to 19, wherein the ratio of the mature IPSC-derived neurons and the mature IPSC-derived glia is selected to mimic the ratio of neurons and glia found in a brain region of interest.

21. The method of any one of claims 1 to 20, wherein the numbers of the mature IPSC-derived neurons and the mature IPSC-derived glia in the assembled three-dimensional organoid are selected to mimic numbers of neurons and glia found in a brain region of interest.

22. The method of claim 20 or 21, wherein the mature IPSC-derived neurons and the mature IPSC-derived glia comprise types of neurons and glia found in the same brain region of interest.

23. The method of any one of claims 20 to 22, wherein the brain region of interest is in the basal ganglia, striatum, medulla, pons, midbrain, medulla oblongata, hypothalamus, thalamus, epithalamus, amygdala, superior colliculus, cerebral cortex, neocortex, allocortex, hippocampus, claustrum, olfactory bulb, frontal lobe, temporal lobe, parietal lobe, occipital lobe, caudate-putamen, external globus pallidus, internal globus pallidus, subthalamic nucleus, substantia nigra, thalamus, or motor cortex region of the brain.

24. The method of any one of claims 1 to 23, wherein the assembled three-dimensional organoid comprises at least two types of mature IPSC-derived neurons.

25. The method of any one of claims 1 to 24, wherein the assembled three-dimensional organoid comprises at least two types of mature IPSC-derived glia.

26. An assembled three-dimensional organoid produced by the method of any one of claims 1 to 25.

27. A method of screening a candidate agent to determine its effects on neurons and glia, the method comprising: contacting the assembled three-dimensional organoid of claim 26 with the candidate agent, and determining the effects of the agent on morphologic, genetic, or functional parameters.

28. The method of claim 27, wherein the mature IPSC-derived neurons or the mature IPSC-derived glia in the three-dimensional organoid comprise at least one genetic mutation associated with a neurological disorder, a neurodevelopmental disorder, or a neurodegenerative disease.

29. The method of claim 28, wherein said at least one genetic mutation is a GRN mutation associated with frontotemporal dementia or lipofusis.

30. The method of claim 28 or 29, where said at least one genetic mutation results in knockdown or knockout of a GRN gene.

31. The method of any one of claims 27 to 30, wherein the mature IPSC-derived neurons are interneurons, motor neurons, sensory neurons, afferent neurons, efferent neurons, inhibitory neurons, or excitatory neurons, or any combination thereof.

32. The method of claim 31, wherein the mature IPSC-derived neurons are glutamatergic neurons, cholinergic neurons, GABAergic neurons, dopaminergic neurons, serotonergic neurons, or histaminergic neurons, or any combination thereof.

33. The method of any one of claims 27 to 32, wherein the mature IPSC-derived glia are astrocytes, oligodendrocytes, ependymal cells, NG2 glia, or microglia, or any combination thereof.

34. The method of any one of claims 27 to 33, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

35. The method of any one of claims 27 to 34, further comprising using optogenetics to excite or inhibit one or more selected neurons of interest using light.

36. A method of producing an assembled three-dimensional organoid disease model of Parkinson's disease, the method comprising:
a) isolating mature induced pluripotent stem cell (IPSC)-derived dopaminergic neurons from a first cell population, isolating mature IPSC-derived astrocytes from a second cell population, and isolating mature IPSC-derived microglia from a third cell population wherein the mature IPSC-derived dopaminergic neurons, the mature IPSC-derived astrocytes, or the mature IPSC-derived microglia, or a combination thereof, comprise one or more genetic mutations associated with Parkinson's disease;
b) combining a selected number of the mature IPSC-derived dopaminergic neurons, the mature IPSC-derived astrocytes, and the mature IPSC-derived microglia to produce a mixed culture having the mature IPSC-derived dopaminergic neurons, the mature IPSC-derived astrocytes, and the mature IPSC-derived microglia at a selected ratio;
c) aggregating the mature IPSC-derived dopaminergic neurons, the mature IPSC-derived astrocytes, and the mature IPSC-derived microglia; and
d) culturing the aggregated mature IPSC-derived dopaminergic neurons, the mature IPSC-derived astrocytes, and the mature IPSC-derived microglia, wherein the culturing results in generation of the assembled three-dimensional organoid disease model of Parkinson's disease.

37. The method of claim 36, wherein the mature IPSC-derived astrocytes have ventral midbrain astrocyte characteristics.

38. The method of claim 36 or 37, wherein the mature IPSC-derived dopaminergic neurons or the IPSC-derived microglia, or both the mature IPSC-derived dopaminergic neurons and the IPSC-derived microglia have midbrain characteristics.

39. The method of any one of claims 36 to 38, wherein the one or more genetic mutations associated with Parkinson's disease comprise one or more mutations in one or more genes selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C.

40. The method of claim 39, wherein the one or more genetic mutations associated with Parkinson's disease comprise an SNCA A53T mutation, an ATP13A2 c1306 mutation, or a SYNJ1 R219Q mutation.

41. The method of any one of claims 36 to 40, wherein a CRISPR system is used to introduce one or more genetic mutations associated with Parkinson's disease into the genome of the IPSC-derived dopaminergic neurons, the mature IPSC-derived astrocytes, or the mature IPSC-derived microglia, or the IPSCs or progenitor cells from which they are derived.

42. The method of claim 41, wherein the CRISPR system is used to knockdown or knockout a gene selected from SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, and VPS13C in the IPSC-derived dopaminergic neurons, the mature IPSC-derived astrocytes, or the mature IPSC-derived microglia.

43. The method of claim 42, wherein the CRISPR system comprises a guide RNA (gRNA) capable of hybridizing to a target site in a SNCA, PARK3, UCHL1, LRRK2, GIGYF2, HTRA2, EIF4G1, TMEM230, CHCHD2, RIC3, VPS35, PRKN, PINK1, PARK2, PARK7, PARK10, PARK12, PARK16, ATP13A2 (PARK9), PLA2G6, FBXO7, DNAJC6, SYNJ1, or VPS13C gene sequence.

44. The method of any one of claims 36 to 43, wherein the mature IPSC-derived dopaminergic neurons, the mature IPSC-derived astrocytes, or the mature IPSC-derived microglia, or a combination thereof, are generated from IPSCs comprising the one or more genetic mutations associated with Parkinson's disease.

45. The method of any one of claims 36 to 44, further comprising:
a) collecting somatic cells from a patient having one or more genetic mutations associated with Parkinson's disease;
b) generating IPSCs from the somatic cells; and
c) differentiating the IPSCs to produce the first cell population comprising the mature IPSC-derived dopaminergic neurons, the second cell population comprising the mature IPSC-derived astrocytes, or the third cell population comprising the mature IPSC-derived microglia, or a combination thereof.

46. The method of any one of claims 36 to 45, wherein the mature IPSC-derived dopaminergic neurons, the mature IPSC-derived astrocytes, and the mature IPSC-derived microglia are generated from IPSCs derived from cells from the same source.

47. The method of any one of claims 36 to 46, wherein the selected ratio of the mature IPSC-derived dopaminergic neurons to the mature IPSC-derived astrocytes is a 2:1, 1:1, 1:2, 1:3, or 1:4 ratio.

48. The method of any one of claims 36 to 47, wherein said culturing is performed in a non-adherent container.

49. The method of any one of claims 36 to 48, wherein said aggregating comprising centrifuging the mixed culture.

50. The method of any one of claims 36 to 49, wherein the ratio of the mature IPSC-derived dopaminergic neurons, the mature IPSC-derived astrocytes, and the mature IPSC-derived microglia is selected to mimic the ratio of dopaminergic neurons, astrocytes, and microglia found in a midbrain region of interest.

51. The method of any one of claims 36 to 50, wherein the numbers of the mature IPSC-derived dopaminergic neurons, the mature IPSC-derived astrocytes, and the mature IPSC-derived microglia in the assembled three-dimensional organoid are selected to mimic numbers of dopaminergic neurons, astrocytes, and microglia found in a midbrain region of interest.

52. The method of claim 50 or 51, wherein the midbrain region of interest comprises a substantia nigra region.

53. An assembled three-dimensional organoid disease model of Parkinson's disease produced by the method of any one of claims 36 to 52.

54. A method of screening a candidate agent for treatment of Parkinson's disease, the method comprising: contacting the assembled three-dimensional organoid disease model of Parkinson's disease of claim 53 with the candidate agent, and determining the effects of the agent on morphologic, genetic, or functional parameters.

55. The method of claim 54, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

56. The method of claim 54 or 55, further comprising using optogenetics to excite or inhibit one or more selected dopaminergic neurons of interest using light.

57. The method of any one of claims 54 to 56, further comprising measuring levels of dopamine or alpha-synuclein in the assembled three-dimensional organoid in presence and absence of the candidate agent.

58. The method of any one of claims 54 to 57, wherein the candidate agent is an antiglutamatergic agent, a monoamine oxidase inhibitor, a promitochondrial agent, a calcium channel blocker, or a growth factor.

59. A method of producing an assembled three-dimensional organoid disease model of Alzheimer's disease, the method comprising:
a) isolating mature induced pluripotent stem cell (IPSC)-derived neurons from a first cell population and isolating mature IPSC-derived glia from a second cell population, wherein the mature IPSC-derived neurons or the mature IPSC-derived glia, or the combination thereof comprise one or more genetic mutations associated with Alzheimer's disease;
b) combining a selected number of the mature IPSC-derived neurons and the mature IPSC-derived glia to produce a mixed culture having the mature IPSC-derived neurons and the mature IPSC-derived glia at a selected ratio;
c) aggregating the mature IPSC-derived neurons and the mature IPSC-derived glia; and
d) culturing the aggregated mature IPSC-derived neurons and the mature IPSC-derived glia, wherein the culturing results in generation of the assembled three-dimensional organoid disease model of Alzheimer's disease.

60. The method of claim 59, wherein the mature IPSC-derived neurons comprise cholinergic neurons.

61. The method of claim 59 or 60, wherein the mature IPSC-derived glia comprise astrocytes, microglia, NG2 glia, or oligodendrocytes, or any combination thereof.

62. The method of any one of claims 59 to 61, wherein the mature IPSC-derived neurons and the mature IPSC-derived glia have hippocampus, entorhinal cortex, cerebral cortex, neocortex, amygdala, or temporal lobe characteristics.

63. The method of any one of claims 59 to 62, wherein the one or more genetic mutations associated with Alzheimer's disease comprise one or more mutations in one or more genes selected from APP, PSEN1, PSEN2, ABCA7, SORL, APOE, and TREM2.

64. The method of claim 63, wherein the one or more genetic mutations associated with Alzheimer's disease comprise frameshift or missense mutations in APP, PSEN1, PSEN2, ABCA7, SORL, APOE, or TREM2.

65. The method of any one of claims 59 to 64, wherein a CRISPR system is used to introduce one or more genetic mutations associated with Alzheimer's disease into the genome of the IPSC-derived neurons or the mature IPSC-derived glia, or the IPSCs or progenitor cells from which they are derived.

66. The method of claim 65, wherein the CRISPR system is used to knockdown or knockout a gene selected from APP, PSEN1, PSEN2, ABCA7, SORL, APOE, or TREM2 in the IPSC-derived neurons or the mature IPSC-derived glia, or the combination thereof.

67. The method of claim 66, wherein the CRISPR system comprises a guide RNA (gRNA) capable of hybridizing to a target site in an APP, PSEN1, PSEN2, ABCA7, SORL, APOE, or TREM2 gene sequence.

68. The method of claim 67, wherein the CRISPR system is used to introduce a missense or frameshift mutation in APP, PSEN1, or PSEN1.

69. The method of any one of claims 59 to 68, wherein the mature IPSC-derived neurons or the mature IPSC-derived glia are generated from IPSCs comprising the one or more genetic mutations associated with Alzheimer's disease.

70. The method of any one of claims 59 to 69, further comprising:
a) collecting somatic cells from a patient having one or more genetic mutations associated with Alzheimer's disease;
b) generating IPSCs from the somatic cells; and
c) differentiating the IPSCs to produce the first cell population comprising the mature IPSC-derived neurons and the second cell population comprising the mature IPSC-derived glia.

71. The method of claim 70, wherein the patient has one or more mutations in one or more genes selected from APP, PSEN1, PSEN2, ABCA7, SORL, APOE, and TREM2.

72. The method of claim 71, wherein the patient has an APOE E4 allele.

73. The method of claim 71, wherein the patient has a missense or frameshift mutation in APP, PSEN1, or PSEN1.

74. The method of any one of claims 59 to 73, wherein the mature IPSC-derived neurons and the mature IPSC-derived glia are generated from IPSCs derived from cells from the same source.

75. The method of any one of claims 59 to 74, wherein the selected ratio of the mature IPSC-derived neurons to the mature IPSC-derived glia is a 2:1, 1:1, 1:2, 1:3, or 1:4 ratio.

76. The method of any one of claims 59 to 75, wherein said culturing is performed in a non-adherent container.

77. The method of any one of claims 59 to 76, wherein said aggregating comprising centrifuging the mixed culture.

78. The method of any one of claims 59 to 77, wherein the ratio of the mature IPSC-derived neurons and the mature IPSC-derived glia is selected to mimic the ratio of neurons and glia found in a brain region of interest.

79. The method of any one of claims 59 to 78, wherein the numbers of the mature IPSC-derived neurons and the mature IPSC-derived glia in the assembled three-dimensional organoid are selected to mimic numbers of neurons and glia found in a brain region of interest.

80. The method of claim 78 or 79, wherein the brain region of interest comprises a hippocampus, entorhinal cortex, cerebral cortex, neocortex, amygdala, or temporal lobe region.

81. An assembled three-dimensional organoid disease model of Alzheimer's disease produced by the method of any one of claims 59 to 80.

82. A method of screening a candidate agent for treatment of Alzheimer's disease, the method comprising: contacting the assembled three-dimensional organoid disease model of Alzheimer's disease of claim 81 with the candidate agent, and determining the effects of the agent on morphologic, genetic, or functional parameters.

83. The method of claim 82, wherein determining the effect of the agent comprises performing immunohistochemistry, gene expression profiling, confocal microscopy, atomic force microscopy, super-resolution microcopy, light-sheet microscopy, two-photon microscopy, fluorescence microscopy, calcium imaging, electrophysiology measurements, patch clamping, migration assays, axonal growth and pathfinding assays, or phagocytosis assays.

84. The method of claim 82 or 83, further comprising using optogenetics to excite or inhibit one or more selected neurons of interest using light.

85. The method of any one of claims 82 to 84, further comprising measuring levels of amyloid-beta, tau, hyperphosphorylated tau, presenilins, or acetylcholine in the assembled three-dimensional organoid in presence and absence of the candidate agent.

86. The method of any one of claims 82 to 85, further comprising measuring neurofibrillary tangles inside cell bodies of the mature IPSC-derived neurons of the assembled three-dimensional organoid.

87. The method of any one of claims 82 to 86, further comprising measuring amyloid plaques in the assembled three-dimensional organoid.

88. The method of any one of claims 82 to 87, wherein the candidate agent is a acetylcholinesterase inhibitor or an N-methyl-D-aspartate (NMDA) receptor antagonist.
